SEQUENCE LISTING (622 sequences)

SEQ ID NO: 1 - RNA, 22 nt, mouse
caagcucgugucuguggguccg

SEQ ID NO: 2 - RNA, 21 nt, mouse
ugcaccaugguugucugagca

SEQ ID NO: 3 - RNA, 22 nt, mouse
uguaaacauccucgacuggaag

SEQ ID NO: 4 - RNA, 22 nt, mouse
ggagaauguaguguuaccguga

SEQ ID NO: 5 - RNA, 22 nt, mouse
aaagugcauccauuuuguuugu

SEQ ID NO: 6 - RNA, 22 nt, mouse
caucuuaccggacagugcugga

SEQ ID NO: 7 - RNA, 22 nt, mouse
uggaauguaaagaaguauguau

SEQ ID NO: 8 - RNA, 22 nt, mouse
uauagggauuggagccguggcg

SEQ ID NO: 9 - RNA, 22 nt, mouse
uauucagauuggugccugucau

SEQ ID NO: 10 - RNA, 19 nt, mouse
cugaaaauguugccugaag

SEQ ID NO: 11 - RNA, 22 nt, mouse
cacagcucccaucucagaacaa

SEQ ID NO: 12 - RNA, 21 nt, mouse
uauacauacacacccauauac

SEQ ID NO: 13 - RNA, 22 nt, mouse
gcucugacuagguugcacuacu

SEQ ID NO: 14 - RNA, 23 nt, mouse
accuuggcucuagacugcuuacu

SEQ ID NO: 15 - RNA, 22 nt, mouse
agugguucuugacaguucaaca

SEQ ID NO: 16 - RNA, 22 nt, mouse
caucuuacugggcagcauugga

SEQ ID NO: 17 - RNA, 22 nt, mouse
uaacacugucugguaacgaugu

SEQ ID NO: 18 - RNA, 24 nt, mouse
ugcagcuguuaaggaugguggacu

SEQ ID NO: 19 - RNA, 22 nt, mouse
cugguacaggccugggggauag

SEQ ID NO: 20 - RNA, 22 nt, mouse
uguaaacauccccgacuggaag

SEQ ID NO: 21 - RNA, 24 nt, mouse
agggacgggacguggugcaguguu

SEQ ID NO: 22 - RNA, 21 nt, mouse
uacucagauugauaugaguca

SEQ ID NO: 23 - RNA, 22 nt, mouse
uaucuaguuggaugucaagaca

SEQ ID NO: 24 - RNA, 22 nt, mouse
gcaugacaccacacuggguaga

SEQ ID NO: 25 - RNA, 23 nt, mouse
ugacuggcaccauucuggauaau

SEQ ID NO: 26 - RNA, 23 nt, mouse
ccccucaggccaccagagcccgg

SEQ ID NO: 27 - RNA, 23 nt, mouse
ugagagaugccauucuauguaga

SEQ ID NO: 28 - RNA, 19 nt, mouse
ugcacugaaggcacacagc

SEQ ID NO: 29 - RNA, 22 nt, mouse
uaaggcuccuuccugugcuugc

SEQ ID NO: 30 - RNA, 19 nt, mouse
aguuuucccuucaagucaa

SEQ ID NO: 31 - RNA, 23 nt, mouse
cauauacauacacacacacguau

SEQ ID NO: 32 - RNA, 22 nt, mouse
auauacauacacacccauauac

SEQ ID NO: 33 - RNA, 23 nt, mouse
acauaacauacacacacauguau

SEQ ID NO: 34 - RNA, 22 nt, mouse
agcgggcacagcugugagagcc

SEQ ID NO: 35 - RNA, 22 nt, mouse
auacaguuguucaaccaguuac

SEQ ID NO: 36 - RNA, 21 nt, mouse
uaaccuguugaacaacugaac

SEQ ID NO: 37 - RNA, 24 nt, mouse
uggcagcuggcaagucagaaugca

SEQ ID NO: 38 - RNA, 23 nt, mouse
caagggucacccucugacucugu

SEQ ID NO: 39 - RNA, 21 nt, mouse
uuggcagaaagggcagcugug

SEQ ID NO: 40 - RNA, 21 nt, mouse
gcguggggugguggacucagg

SEQ ID NO: 41 - RNA, 19 nt, mouse
ucucccaacccuuuuccca

SEQ ID NO: 42 - RNA, 20 nt, mouse
gguccagugacuaagagcau

SEQ ID NO: 43 - RNA, 20 nt, mouse
ccgcgggacccggggcugug

SEQ ID NO: 44 - RNA, 19 nt, mouse
auugauuguuaagcugaaa

SEQ ID NO: 45 - RNA, 19 nt, mouse
aagcgcaaggcccugggug

SEQ ID NO: 46 - RNA, 20 nt, mouse
uuagaucgauguggugcucc

SEQ ID NO: 47 - RNA, 22 nt, mouse
agacccuggucugcacucuauc

SEQ ID NO: 48 - RNA, 21 nt, mouse
caaaccacacugugguguuag

SEQ ID NO: 49 - RNA, 22 nt, mouse
ucaggcucaguccccucccgau

SEQ ID NO: 50 - RNA, 22 nt, mouse
uacgugugugugcaugugcaug

SEQ ID NO: 51 - RNA, 21 nt, mouse
cauacacacacacauacacac

SEQ ID NO: 52 - RNA, 22 nt, mouse
uaccuaauuuguuguccaucau

SEQ ID NO: 53 - RNA, 22 nt, mouse
cuccccaccccuguaccaguga

SEQ ID NO: 54 - RNA, 22 nt, mouse
gaaguuguucgugguggauucg

SEQ ID NO: 55 - RNA, 22 nt, mouse
gccugcugggguggaaccuggu

SEQ ID NO: 56 - RNA, 21 nt, mouse
agucaggcuccuggcaggagu

SEQ ID NO: 57 - RNA, 22 nt, mouse
gauauaaccacugccagacuga

SEQ ID NO: 58 - RNA, 21 nt, mouse
cggucggccgaucgcucgguc

SEQ ID NO: 59 - RNA, 22 nt, mouse
aacacacccagcuaaccuuuuu

SEQ ID NO: 60 - RNA, 22 nt, mouse
aggugguccguggcgcguucgc

SEQ ID NO: 61 - RNA, 21 nt, mouse
aaacaugaagcgcugcaacac

SEQ ID NO: 62 - RNA, 20 nt, mouse
uccuggcccugguccugguc

SEQ ID NO: 63 - RNA, 20 nt, mouse
cggggcagcucaguacagga

SEQ ID NO: 64 - RNA, 22 nt, mouse
uguaaacauccuugacuggaag

SEQ ID NO: 65 - RNA, 21 nt, mouse
ggccaaggaugagaacucuaa

SEQ ID NO: 66 - RNA, 21 nt, mouse
uguuggggacauuuuuaaagc

SEQ ID NO: 67 - RNA, 22 nt, mouse
aggcugggaauauuucagagau

SEQ ID NO: 68 - RNA, 23 nt, mouse
cgcacucuggucuucccuugcag

SEQ ID NO: 69 - RNA, 22 nt, mouse
guuccugcugaacugagccagu

SEQ ID NO: 70 - RNA, 23 nt, mouse
ucagcaccaggauauuguugggg

SEQ ID NO: 71 - RNA, 23 nt, mouse
ucagccacggcuuaccuggaaga

SEQ ID NO: 72 - RNA, 22 nt, mouse
aaagugcuucccuuuugugugu

SEQ ID NO: 73 - RNA, 19 nt, mouse
ggggccuggcggcgggcgg

SEQ ID NO: 74 - RNA, 22 nt, mouse
agaguugcgucuggacgucccg

SEQ ID NO: 75 - RNA, 22 nt, mouse
aaacaugguuccgucaagcacc

SEQ ID NO: 76 - RNA, 21 nt, mouse
ucugucauucuguaggccaau

SEQ ID NO: 77 - RNA, 23 nt, mouse
caaagugcucauagugcagguag

SEQ ID NO: 78 - RNA, 22 nt, mouse
uggaauguaaggaagugugugg

SEQ ID NO: 79 - RNA, 22 nt, mouse
uccuucauuccaccggagucug

SEQ ID NO: 80 - RNA, 23 nt, mouse
uggguacauaaagaaguaugugc

SEQ ID NO: 81 - RNA, 22 nt, mouse
aguccagggcugagucagcgga

SEQ ID NO: 82 - RNA, 22 nt, mouse
ugggaaaguucucaggcuucug

SEQ ID NO: 83 - RNA, 22 nt, mouse
aacuggcccacaaagucccgcu

SEQ ID NO: 84 - RNA, 22 nt, mouse
ggugcaguuacuguggcugugg

SEQ ID NO: 85 - RNA, 22 nt, mouse
agcgauggccgaaucugcuucc

SEQ ID NO: 86 - RNA, 22 nt, mouse
aggucaagguucacaggggauc

SEQ ID NO: 87 - RNA, 22 nt, mouse
cucucugauggugggugaggag

SEQ ID NO: 88 - RNA, 23 nt, mouse
uuaaugcuaauugugauaggggu

SEQ ID NO: 89 - RNA, 21 nt, mouse
cuccuaccuguuagcauuaac

SEQ ID NO: 90 - RNA, 23 nt, mouse
ucuggcuccgugucuucacuccc

SEQ ID NO: 91 - RNA, 21 nt, mouse
ggugcagugcugcaucucugg

SEQ ID NO: 92 - RNA, 22 nt, mouse
caucuuccagugcaguguugga

SEQ ID NO: 93 - RNA, 22 nt, mouse
acuccauuuguuuugaugaugg

SEQ ID NO: 94 - RNA, 22 nt, mouse
aucaucgucucaaaugagucuu

SEQ ID NO: 95 - RNA, 22 nt, mouse
acccgucccguucguccccgga

SEQ ID NO: 96 - RNA, 22 nt, mouse
ucagcugagguuccccucuguc

SEQ ID NO: 97 - RNA, 23 nt, mouse
acugcagugccagcacuucuuac

SEQ ID NO: 98 - RNA, 22 nt, mouse
cuauacaaucuacugucuuucc

SEQ ID NO: 99 - RNA, 22 nt, mouse
uauaccucaguuuuaucaggug

SEQ ID NO: 100 - RNA, 22 nt, mouse
ucaguaacaaagauucauccuu

SEQ ID NO: 101 - RNA, 22 nt, mouse
caacuagacugugagcuucuag

SEQ ID NO: 102 - RNA, 21 nt, mouse
cagccucgcuggcaggcagcu

SEQ ID NO: 103 - RNA, 22 nt, mouse
uucagugaugauuagcuucuga

SEQ ID NO: 104 - RNA, 22 nt, mouse
cuguaugcccuaaccgcucagu

SEQ ID NO: 105 - RNA, 22 nt, mouse
auauacauacacacccauauau

SEQ ID NO: 106 - RNA, 22 nt, mouse
ugucuugugugugcauguucau

SEQ ID NO: 107 - RNA, 22 nt, mouse
cauacaaggauaauuucuuuuu

SEQ ID NO: 108 - RNA, 24 nt, mouse
uguagauccauaugccauggugug

SEQ ID NO: 109 - RNA, 19 nt, mouse
cuggacgcgagcugggccc

SEQ ID NO: 110 - RNA, 20 nt, mouse
ggcgccgcucguggggggcc

SEQ ID NO: 111 - RNA, 24 nt, mouse
cugugcuagugagguggcucagca

SEQ ID NO: 112 - RNA, 22 nt, mouse
gaguauuguuuccacugccugg

SEQ ID NO: 113 - RNA, 25 nt, mouse
ugucauaugugugaugacacuuucu

SEQ ID NO: 114 - RNA, 22 nt, mouse
agucauacacggcucuccucuc

SEQ ID NO: 115 - RNA, 21 nt, mouse
ucacuccuccccucccgucuu

SEQ ID NO: 116 - RNA, 22 nt, mouse
auauacauacacacaccuauac

SEQ ID NO: 117 - RNA, 20 nt, mouse
gagguuguaguuugugcuuu

SEQ ID NO: 118 - RNA, 20 nt, mouse
cccuaaaguagaaaucacua

SEQ ID NO: 119 - RNA, 22 nt, mouse
uauacaagggcaagcucucugu

SEQ ID NO: 120 - RNA, 22 nt, mouse
guggauauuccuucuauguuua

SEQ ID NO: 121 - RNA, 22 nt, mouse
gcgacgagccccucgcacaaac

SEQ ID NO: 122 - RNA, 20 nt, mouse
caggucacgucucugcaguu

SEQ ID NO: 123 - RNA, 22 nt, mouse
aauugcacgguauccaucugua

SEQ ID NO: 124 - RNA, 22 nt, mouse
uucacaaagcccauacacuuuc

SEQ ID NO: 125 - RNA, 22 nt, mouse
gguauaaccaaagcccgacugu

SEQ ID NO: 126 - RNA, 23 nt, mouse
gcaaagcacagggccugcagaga

SEQ ID NO: 127 - RNA, 21 nt, mouse
uucugccuccccugaaggcuc

SEQ ID NO: 128 - RNA, 22 nt, mouse
uguaaacauccuacacucagcu

SEQ ID NO: 129 - RNA, 22 nt, mouse
uggugcuaccgucagggguaga

SEQ ID NO: 130 - RNA, 22 nt, mouse
ucccacaggcccagcucauagc

SEQ ID NO: 131 - RNA, 22 nt, mouse
aaauggugcccuagugacuaca

SEQ ID NO: 132 - RNA, 23 nt, mouse
uaaagugcuuauagugcagguag

SEQ ID NO: 133 - RNA, 17 nt, mouse
ugagguaguaguuagaa

SEQ ID NO: 134 - RNA, 21 nt, mouse
ucagaugucuucaucugguug

SEQ ID NO: 135 - RNA, 22 nt, mouse
augcaagggcuggugcgauggc

SEQ ID NO: 136 - RNA, 22 nt, mouse
ccuucuucuucuuccugagaca

SEQ ID NO: 137 - RNA, 22 nt, mouse
ccgcucguacucccgggggucc

SEQ ID NO: 138 - RNA, 20 nt, mouse
ccgaucguuccccuccauac

SEQ ID NO: 139 - RNA, 22 nt, mouse
aaaguucugagacacuccgacu

SEQ ID NO: 140 - RNA, 22 nt, mouse
acucuuucccuguugcacuacu

SEQ ID NO: 141 - RNA, 22 nt, mouse
caaaucuuauuugagcaccugu

SEQ ID NO: 142 - RNA, 22 nt, mouse
gaaugaguaacugcuagauccu

SEQ ID NO: 143 - RNA, 25 nt, mouse
uccgaggcuccccaccacacccugc

SEQ ID NO: 144 - RNA, 22 nt, mouse
ucccugagacccuaacuuguga

SEQ ID NO: 145 - RNA, 22 nt, mouse
ucguaccgugaguaauaaugcg

SEQ ID NO: 146 - RNA, 21 nt, mouse
uucacaguggcuaaguuccgc

SEQ ID NO: 147 - RNA, 22 nt, mouse
cugggagaggguuguuuacucc

SEQ ID NO: 148 - RNA, 22 nt, mouse
aacccguagauccgaucuugug

SEQ ID NO: 149 - RNA, 21 nt, mouse
caagcucguuucuaugggucu

SEQ ID NO: 150 - RNA, 23 nt, mouse
caaagugcuguucgugcagguag

SEQ ID NO: 151 - RNA, 22 nt, mouse
uauugcacucgucccggccucc

SEQ ID NO: 152 - RNA, 22 nt, mouse
gcuuauggcuucaagcuuucgg

SEQ ID NO: 153 - RNA, 21 nt, mouse
aagguuacuuguuaguucagg

SEQ ID NO: 154 - RNA, 23 nt, mouse
uauucagauuagugccagucaug

SEQ ID NO: 155 - RNA, 19 nt, mouse
ggugcucacauguccuccu

SEQ ID NO: 156 - RNA, 20 nt, mouse
cggcucugggucugugggga

SEQ ID NO: 157 - RNA, 21 nt, mouse
cagugcaauuaaaagggggaa

SEQ ID NO: 158 - RNA, 18 nt, mouse
aucucgcuggggccucca

SEQ ID NO: 159 - RNA, 22 nt, mouse
cucagacagagauaccuucucu

SEQ ID NO: 160 - RNA, 21 nt, mouse
cuccuucacccgggcgguacc

SEQ ID NO: 161 - RNA, 22 nt, mouse
gggacccggggagagauguaag

SEQ ID NO: 162 - RNA, 19 nt, mouse
ggaggcagaggcaggagga

SEQ ID NO: 163 - RNA, 21 nt, mouse
ugcccacccuuuaccccgcuc

SEQ ID NO: 164 - RNA, 21 nt, mouse
ccguccugagguuguugagcu

SEQ ID NO: 165 - RNA, 22 nt, mouse
acuugugugugcauguauaugu

SEQ ID NO: 166 - RNA, 22 nt, mouse
auaguuguguguggaugugugu

SEQ ID NO: 167 - RNA, 23 nt, mouse
auugugucaauaugcgaugaugu

SEQ ID NO: 168 - RNA, 22 nt, mouse
gagcuuauucauaaaagugcag

SEQ ID NO: 169 - RNA, 21 nt, mouse
uaauuuuauguauaagcuagu

SEQ ID NO: 170 - RNA, 23 nt, mouse
ugagugugugugugugagugugu

SEQ ID NO: 171 - RNA, 20 nt, mouse
auguauaaauguauacacac

SEQ ID NO: 172 - RNA, 22 nt, mouse
gccccaucgauuaacugcuucc

SEQ ID NO: 173 - RNA, 22 nt, mouse
uucaccacacccagcuuaaaga

SEQ ID NO: 174 - RNA, 23 nt, mouse
aggagguccuggggccgcccuga

SEQ ID NO: 175 - RNA, 23 nt, mouse
gaaaucaagcuugggugagaccu

SEQ ID NO: 176 - RNA, 21 nt, mouse
gcgacccauacuugguuucag

SEQ ID NO: 177 - RNA, 21 nt, mouse
ucuuguuaaaaagcagagucu

SEQ ID NO: 178 - RNA, 22 nt, mouse
aaacauucgcggugcacuucuu

SEQ ID NO: 179 - RNA, 22 nt, mouse
cucggggaucaucaugucacga

SEQ ID NO: 180 - RNA, 20 nt, mouse
auaugcgagggaacuacugg

SEQ ID NO: 181 - RNA, 23 nt, mouse
guuagugaugaucaauaaaguua

SEQ ID NO: 182 - RNA, 22 nt, mouse
acuggagacggaagcugcaaga

SEQ ID NO: 183 - RNA, 23 nt, mouse
aggucuguagcucaguuggcaga

SEQ ID NO: 184 - RNA, 21 nt, mouse
ucgaaucccagcggugccucu

SEQ ID NO: 185 - RNA, 24 nt, mouse
guucaugucccuguucaggcgcca

SEQ ID NO: 186 - RNA, 24 nt, mouse
agcucccgccacugugacccccuu

SEQ ID NO: 187 - RNA, 22 nt, mouse
cuuaugcaagauucccuucuac

SEQ ID NO: 188 - RNA, 21 nt, mouse
cccagauaauagcacucucaa

SEQ ID NO: 189 - RNA, 21 nt, mouse
uugaaaggcuguuucuugguc

SEQ ID NO: 190 - RNA, 22 nt, mouse
uaagugcgcgcauguauaugcg

SEQ ID NO: 191 - RNA, 24 nt, mouse
agguugccucauagugagcuugca

SEQ ID NO: 192 - RNA, 22 nt, mouse
uguuugcagaggaaacugagac

SEQ ID NO: 193 - RNA, 21 nt, mouse
ugucuugcaggccgucaugca

SEQ ID NO: 194 - RNA, 22 nt, mouse
uaauacugucugguaaugccgu

SEQ ID NO: 195 - RNA, 23 nt, mouse
ugaggggcagagagcgagacuuu

SEQ ID NO: 196 - RNA, 23 nt, mouse
aucaacagacauuaauugggcgc

SEQ ID NO: 197 - RNA, 22 nt, mouse
uauguaacaugguccacuaacu

SEQ ID NO: 198 - RNA, 22 nt, mouse
guggauauuccuucuaugguua

SEQ ID NO: 199 - RNA, 22 nt, mouse
aauugcacuuuagcaaugguga

SEQ ID NO: 200 - RNA, 23 nt, mouse
agggacuuucaggggcagcugug

SEQ ID NO: 201 - RNA, 21 nt, mouse
ggucaagaggcgccugggaac

SEQ ID NO: 202 - RNA, 21 nt, mouse
ucacuuuguagaccaggcugg

SEQ ID NO: 203 - RNA, 24 nt, mouse
agucaggcugcuggcuauacacca

SEQ ID NO: 204 - RNA, 22 nt, mouse
aggggugcuaucugugauugag

SEQ ID NO: 205 - RNA, 22 nt, mouse
uccgucucaguuacuuuauagc

SEQ ID NO: 206 - RNA, 22 nt, mouse
caauguuuccacagugcaucac

SEQ ID NO: 207 - RNA, 22 nt, mouse
ucucugggccugugucuuaggc

SEQ ID NO: 208 - RNA, 22 nt, mouse
aggcaagaugcuggcauagcug

SEQ ID NO: 209 - RNA, 22 nt, mouse
gcacuccaucggaggcagacac

SEQ ID NO: 210 - RNA, 21 nt, mouse
uagggccaucucauccagaua

SEQ ID NO: 211 - RNA, 23 nt, mouse
acgcucugcuuugcucccccaga

SEQ ID NO: 212 - RNA, 21 nt, mouse
cucuacucccugccccagcca

SEQ ID NO: 213 - RNA, 23 nt, mouse
uugggaacggggugucuuuggga

SEQ ID NO: 214 - RNA, 21 nt, mouse
cugugacacacccgcucccag

SEQ ID NO: 215 - RNA, 22 nt, mouse
aagcuuucucaucugugacacu

SEQ ID NO: 216 - RNA, 21 nt, mouse
ccuuuaaauuguguccucaag

SEQ ID NO: 217 - RNA, 22 nt, mouse
uguggacaccgugggagguugg

SEQ ID NO: 218 - RNA, 22 nt, mouse
ugaguucagggacagcgugucu

SEQ ID NO: 219 - RNA, 23 nt, mouse
uucaugagcagcugcaaaggugu

SEQ ID NO: 220 - RNA, 23 nt, mouse
gacuggagcuuggagcggugagc

SEQ ID NO: 221 - RNA, 24 nt, mouse
cacaggggaagcucagugccagcc

SEQ ID NO: 222 - RNA, 21 nt, mouse
auccuuggccuuccuaggugu

SEQ ID NO: 223 - RNA, 22 nt, mouse
cuggcgccaacugugaccacug

SEQ ID NO: 224 - RNA, 22 nt, mouse
agggaccccgagggagggcagg

SEQ ID NO: 225 - RNA, 22 nt, mouse
aucaucaaaacaaauggagucc

SEQ ID NO: 226 - RNA, 23 nt, mouse
aguucucaggcccgcuguggugu

SEQ ID NO: 227 - RNA, 22 nt, mouse
cacuuaauagaccgcaaccugc

SEQ ID NO: 228 - RNA, 22 nt, mouse
ucaacaaaaucacugaugcugg

SEQ ID NO: 229 - RNA, 19 nt, mouse
cggggacacacuuucuccu

SEQ ID NO: 230 - RNA, 22 nt, mouse
cagugggccgugaaagguagcc

SEQ ID NO: 231 - RNA, 22 nt, mouse
uuuccucucugccccauagggu

SEQ ID NO: 232 - RNA, 23 nt, mouse
uaagugcuuccauguuuuaguag

SEQ ID NO: 233 - RNA, 22 nt, mouse
acuuaaacgugguuguacuugc

SEQ ID NO: 234 - RNA, 23 nt, mouse
uaagugcuuccauguuuugguga

SEQ ID NO: 235 - RNA, 22 nt, mouse
gcucugacuuuauugcacuacu

SEQ ID NO: 236 - RNA, 22 nt, mouse
uugaagagagguuauccuuugu

SEQ ID NO: 237 - RNA, 22 nt, mouse
gcugguuucauauggugguuua

SEQ ID NO: 238 - RNA, 22 nt, mouse
uagcaccaucugaaaucgguua

SEQ ID NO: 239 - RNA, 22 nt, mouse
uaugugggacgguaaaccgcuu

SEQ ID NO: 240 - RNA, 22 nt, mouse
acucaaaauggaggcccuaucu

SEQ ID NO: 241 - RNA, 22 nt, mouse
acucaaacugugugacauuuug

SEQ ID NO: 242 - RNA, 22 nt, mouse
agugccgcagaguuuguagugu

SEQ ID NO: 243 - RNA, 22 nt, mouse
gggguuccuggggaugggauuu

SEQ ID NO: 244 - RNA, 21 nt, mouse
uaagucacuagugguuccguu

SEQ ID NO: 245 - RNA, 22 nt, mouse
ugucaguuugucaaauacccca

SEQ ID NO: 246 - RNA, 21 nt, mouse
ugauuguccaaacgcaauucu

SEQ ID NO: 247 - RNA, 23 nt, mouse
caucaguuccuaaugcauugccu

SEQ ID NO: 248 - RNA, 21 nt, mouse
gcaaggacagcaaaggggggc

SEQ ID NO: 249 - RNA, 22 nt, mouse
aagcuuuuugcucgcguuaugu

SEQ ID NO: 250 - RNA, 22 nt, mouse
auaagacgagcaaaaagcuugu

SEQ ID NO: 251 - RNA, 22 nt, mouse
cgucuuacccagcaguguuugg

SEQ ID NO: 252 - RNA, 23 nt, mouse
ugugcaaaucuaugcaaaacuga

SEQ ID NO: 253 - RNA, 22 nt, mouse
ucggcaacaagaaacugccuga

SEQ ID NO: 254 - RNA, 25 nt, mouse
aagggagcuggcucaggagagaguc

SEQ ID NO: 255 - RNA, 23 nt, mouse
ugggacgagaucaugaggccuuc

SEQ ID NO: 256 - RNA, 17 nt, mouse
ucuccacccuccuucug

SEQ ID NO: 257 - RNA, 22 nt, mouse
ccaguggagcugcuguuacuuc

SEQ ID NO: 258 - RNA, 22 nt, mouse
cgggguuuugagggcgagauga

SEQ ID NO: 259 - RNA, 22 nt, mouse
aggcagaggcuggcggaucucu

SEQ ID NO: 260 - RNA, 23 nt, mouse
ucugguccccugcuucguccucu

SEQ ID NO: 261 - RNA, 22 nt, mouse
ccaggaccaucagugugacuau

SEQ ID NO: 262 - RNA, 22 nt, mouse
agaggugcaguaggcaugacuu

SEQ ID NO: 263 - RNA, 23 nt, mouse
uaaggugcaucuagugcagauag

SEQ ID NO: 264 - RNA, 22 nt, mouse
cucuccccuaccaccugccucu

SEQ ID NO: 265 - RNA, 21 nt, mouse
cucccacaugcaggguuugca

SEQ ID NO: 266 - RNA, 21 nt, mouse
gugguucuagacuugccaacu

SEQ ID NO: 267 - RNA, 22 nt, mouse
ucagugcacuacagaacuuugu

SEQ ID NO: 268 - RNA, 20 nt, mouse
cugugggccaccuagucacc

SEQ ID NO: 269 - RNA, 22 nt, mouse
aaccguggcuuucgauuguuac

SEQ ID NO: 270 - RNA, 22 nt, mouse
uaacagucuacagccauggucg

SEQ ID NO: 271 - RNA, 23 nt, mouse
gggggccgaugcacuguaagaga

SEQ ID NO: 272 - RNA, 21 nt, mouse
cggggccguagcacugucuga

SEQ ID NO: 273 - RNA, 22 nt, mouse
cugaagcucagagggcucugau

SEQ ID NO: 274 - RNA, 22 nt, mouse
ucggauccgucugagcuuggcu

SEQ ID NO: 275 - RNA, 21 nt, mouse
cgcuuugcucagccaguguag

SEQ ID NO: 276 - RNA, 23 nt, mouse
ucugggcagagcugcaggagaga

SEQ ID NO: 277 - RNA, 21 nt, mouse
cgguugaccaugguguguacg

SEQ ID NO: 278 - RNA, 20 nt, mouse
aaaucuaccugccucugccu

SEQ ID NO: 279 - RNA, 20 nt, mouse
ugguagaccggugacguaca

SEQ ID NO: 280 - RNA, 21 nt, mouse
cagucuuacuauguagcccua

SEQ ID NO: 281 - RNA, 23 nt, mouse
agcuucuuuacagugcugccuug

SEQ ID NO: 282 - RNA, 22 nt, mouse
ucaguuaucacagugcugaugc

SEQ ID NO: 283 - RNA, 22 nt, mouse
acaagcuugugucuauagguau

SEQ ID NO: 284 - RNA, 22 nt, mouse
acuguacaggccacugccuugc

SEQ ID NO: 285 - RNA, 22 nt, mouse
ugagguaggagguuguauaguu

SEQ ID NO: 286 - RNA, 23 nt, mouse
agcagcauuguacagggcuauca

SEQ ID NO: 287 - RNA, 23 nt, mouse
agcuucuuuacaguguugccuug

SEQ ID NO: 288 - RNA, 22 nt, mouse
caaauucguaucuaggggaaua

SEQ ID NO: 289 - RNA, 23 nt, mouse
uacccuguagaaccgaauuugug

SEQ ID NO: 290 - RNA, 22 nt, mouse
accaucgaccguugauuguacc

SEQ ID NO: 291 - RNA, 23 nt, mouse
caacggaaucccaaaagcagcug

SEQ ID NO: 292 - RNA, 24 nt, mouse
aguuuugcagauuugcaguucagc

SEQ ID NO: 293 - RNA, 23 nt, mouse
agcuacauugucugcuggguuuc

SEQ ID NO: 294 - RNA, 23 nt, mouse
uguaaacauccuacacucucagc

SEQ ID NO: 295 - RNA, 22 nt, mouse
aggcaggggcugggccugcagc

SEQ ID NO: 296 - RNA, 23 nt, mouse
ucuuugguuaucuagcuguauga

SEQ ID NO: 297 - RNA, 22 nt, mouse
uuugguccccuucaaccagcug

SEQ ID NO: 298 - RNA, 22 nt, mouse
ugugacugguugaccagagggg

SEQ ID NO: 299 - RNA, 23 nt, mouse
uauggcuuuuuauuccuauguga

SEQ ID NO: 300 - RNA, 23 nt, mouse
uuauugcuuaagaauacgcguag

SEQ ID NO: 301 - RNA, 22 nt, mouse
uagguuauccguguugccuucg

SEQ ID NO: 302 - RNA, 22 nt, mouse
uauggcacugguagaauucacu

SEQ ID NO: 303 - RNA, 22 nt, mouse
uggagagaaaggcaguuccuga

SEQ ID NO: 304 - RNA, 22 nt, mouse
cuauacgaccugcugccuuucu

SEQ ID NO: 305 - RNA, 22 nt, mouse
uagcaccauuugaaaucgguua

SEQ ID NO: 306 - RNA, 21 nt, mouse
uucagcuccuauaugaugccu

SEQ ID NO: 307 - RNA, 22 nt, mouse
aaggagcucacagucuauugag

SEQ ID NO: 308 - RNA, 21 nt, mouse
uugugcuugaucuaaccaugu

SEQ ID NO: 309 - RNA, 21 nt, mouse
gugcauuguaguugcauugca

SEQ ID NO: 310 - RNA, 22 nt, mouse
cuccugacuccagguccugugu

SEQ ID NO: 311 - RNA, 21 nt, mouse
aauauaacacagauggccugu

SEQ ID NO: 312 - RNA, 20 nt, mouse
aggucagaggucgauccugg

SEQ ID NO: 313 - RNA, 22 nt, mouse
aaaggcuaggcucacaaccaaa

SEQ ID NO: 314 - RNA, 21 nt, mouse
gcugguaaaauggaaccaaau

SEQ ID NO: 315 - RNA, 22 nt, mouse
cucacagcucugguccuuggag

SEQ ID NO: 316 - RNA, 22 nt, mouse
ugcggggcuagggcuaacagca

SEQ ID NO: 317 - RNA, 22 nt, mouse
gugaauuaccgaagggccauaa

SEQ ID NO: 318 - RNA, 22 nt, mouse
acugauuucuuuugguguucag

SEQ ID NO: 319 - RNA, 22 nt, mouse
aacaauauccuggugcugagug

SEQ ID NO: 320 - RNA, 22 nt, mouse
uauguguguguacauguacaua

SEQ ID NO: 321 - RNA, 22 nt, mouse
aggagagaguuagcgcauuagu

SEQ ID NO: 322 - RNA, 22 nt, mouse
gauguguguguacauguacaua

SEQ ID NO: 323 - RNA, 21 nt, mouse
auacagacacaugcacacaca

SEQ ID NO: 324 - RNA, 23 nt, mouse
ugugugcaugugcauguguguaa

SEQ ID NO: 325 - RNA, 21 nt, mouse
uauacauacacacacauauau

SEQ ID NO: 326 - RNA, 22 nt, mouse
ugcagcagccugaggcagggcu

SEQ ID NO: 327 - RNA, 22 nt, mouse
guucugcuccucuggagggagg

SEQ ID NO: 328 - RNA, 23 nt, mouse
aagggaggaucugggcaccugga

SEQ ID NO: 329 - RNA, 22 nt, mouse
agaggcuggcacugggacacau

SEQ ID NO: 330 - RNA, 22 nt, mouse
aagguagauagaacaggucuug

SEQ ID NO: 331 - RNA, 22 nt, mouse
ucuggcuguuguggugugcaaa

SEQ ID NO: 332 - RNA, 22 nt, mouse
ggugaauugcaguacuccaaca

SEQ ID NO: 333 - RNA, 22 nt, mouse
uugauguccacugugaccauag

SEQ ID NO: 334 - RNA, 21 nt, mouse
caugggucugguugggcccgc

SEQ ID NO: 335 - RNA, 22 nt, mouse
uccuaacagcaggaguaggagc

SEQ ID NO: 336 - RNA, 23 nt, mouse
agucaggcugcuggcuagaguac

SEQ ID NO: 337 - RNA, 23 nt, mouse
gagcaccccauuggcuacccaca

SEQ ID NO: 338 - RNA, 25 nt, mouse
uagggggcaggagccggagcccucu

SEQ ID NO: 339 - RNA, 21 nt, mouse
caguuugucucuucuuugucu

SEQ ID NO: 340 - RNA, 22 nt, mouse
cugccaauuccauaggucacag

SEQ ID NO: 341 - RNA, 23 nt, mouse
ggcuucuuuacagugcugccuug

SEQ ID NO: 342 - RNA, 21 nt, mouse
uguagggauggaagccaugaa

SEQ ID NO: 343 - RNA, 22 nt, mouse
ucagucucaucugcaaagaggu

SEQ ID NO: 344 - RNA, 24 nt, mouse
aguugugugugcaugugcaugugu

SEQ ID NO: 345 - RNA, 21 nt, mouse
auaugaguauucugccuaaau

SEQ ID NO: 346 - RNA, 22 nt, mouse
agcuggagcacaaaagccggug

SEQ ID NO: 347 - RNA, 20 nt, mouse
aaaggauuuaccugaggcca

SEQ ID NO: 348 - RNA, 21 nt, mouse
cgaaucccacuccagacacca

SEQ ID NO: 349 - RNA, 22 nt, mouse
uuuguuuguuuugcugaugcag

SEQ ID NO: 350 - RNA, 22 nt, mouse
auacgcacacuuaagacuuuag

SEQ ID NO: 351 - RNA, 23 nt, mouse
agccccugaccuugaaccuggga

SEQ ID NO: 352 - RNA, 23 nt, mouse
ugugugcaugugcauguguguau

SEQ ID NO: 353 - RNA, 22 nt, mouse
agagaaacccugucucaaaaaa

SEQ ID NO: 354 - RNA, 22 nt, mouse
ugagguaguaguuugugcuguu

SEQ ID NO: 355 - RNA, 21 nt, mouse
uacaguacugugauaacugaa

SEQ ID NO: 356 - RNA, 24 nt, mouse
ucccugagacccuuuaaccuguga

SEQ ID NO: 357 - RNA, 21 nt, mouse
ucagugcaugacagaacuugg

SEQ ID NO: 358 - RNA, 22 nt, mouse
uacucaguaaggcauuguucuu

SEQ ID NO: 359 - RNA, 22 nt, mouse
agagguauagcgcaugggaaga

SEQ ID NO: 360 - RNA, 22 nt, mouse
acucaaacuaugggggcacuuu

SEQ ID NO: 361 - RNA, 23 nt, mouse
aggcaguguaguuagcugauugc

SEQ ID NO: 362 - RNA, 22 nt, mouse
ugagguaguagguugugugguu

SEQ ID NO: 363 - RNA, 24 nt, mouse
ucccugaggagcccuuugagccug

SEQ ID NO: 364 - RNA, 23 nt, mouse
uauggcuuuucauuccuauguga

SEQ ID NO: 365 - RNA, 22 nt, mouse
aacauucaaccugucggugagu

SEQ ID NO: 366 - RNA, 23 nt, mouse
uacugcaucaggaacugacugga

SEQ ID NO: 367 - RNA, 22 nt, mouse
uauguaguaugguccacaucuu

SEQ ID NO: 368 - RNA, 21 nt, mouse
augaccuaugauuugacagac

SEQ ID NO: 369 - RNA, 22 nt, mouse
uugcauauguaggaugucccau

SEQ ID NO: 370 - RNA, 22 nt, mouse
uggcaguguauuguuagcuggu

SEQ ID NO: 371 - RNA, 21 nt, mouse
cuugguacaucuuugagugag

SEQ ID NO: 372 - RNA, 22 nt, mouse
ugaaacauacacgggaaaccuc

SEQ ID NO: 373 - RNA, 22 nt, mouse
gcuuuaacaugggguuaccugc

SEQ ID NO: 374 - RNA, 22 nt, mouse
aagugcuuccauguuucagugg

SEQ ID NO: 375 - RNA, 22 nt, mouse
ggacugugaggugacucuuggu

SEQ ID NO: 376 - RNA, 21 nt, mouse
ccugcuguaagcuguguccuc

SEQ ID NO: 377 - RNA, 22 nt, mouse
auugcuucccagacggugaaga

SEQ ID NO: 378 - RNA, 22 nt, mouse
ugagaacugaauuccauaggcu

SEQ ID NO: 379 - RNA, 22 nt, mouse
auauacauacacacaccaacac

SEQ ID NO: 380 - RNA, 22 nt, mouse
uaugugccuuuggacuacaucg

SEQ ID NO: 381 - RNA, 19 nt, mouse
cauucucguuuccuucccu

SEQ ID NO: 382 - RNA, 22 nt, mouse
agagaaacccugucucaaaaaa

SEQ ID NO: 383 - RNA, 21 nt, mouse
cagucaugccgcuugccuacg

SEQ ID NO: 384 - RNA, 21 nt, mouse
cgacgagggccggucggucgc

SEQ ID NO: 385 - RNA, 22 nt, mouse
aaugcacccgggcaaggauuug

SEQ ID NO: 386 - RNA, 22 nt, mouse
auugggaacauuuugcaugcau

SEQ ID NO: 387 - RNA, 22 nt, mouse
cgucaacacuugcugguuuucu

SEQ ID NO: 388 - RNA, 21 nt, mouse
cuuccgcccggccgggugucg

SEQ ID NO: 389 - RNA, 22 nt, mouse
uggugcggaaagggcccacagu

SEQ ID NO: 390 - RNA, 22 nt, mouse
gguuguauuaucauuguccgag

SEQ ID NO: 391 - RNA, 20 nt, mouse
accaggaggcugaggucccu

SEQ ID NO: 392 - RNA, 19 nt, mouse
uuugugaccugguccacua

SEQ ID NO: 393 - RNA, 22 nt, mouse
ccagcugggaagaaccaguggc

SEQ ID NO: 394 - RNA, 21 nt, mouse
uuccuaugcauauacuucuuu

SEQ ID NO: 395 - RNA, 22 nt, mouse
caggccauacugugcugccuca

SEQ ID NO: 396 - RNA, 22 nt, mouse
acugcauuacgagcacuuaaag

SEQ ID NO: 397 - RNA, 23 nt, mouse
ugcuaugccaacauauugccauc

SEQ ID NO: 398 - RNA, 22 nt, mouse
acugcugagcuagcacuucccg

SEQ ID NO: 399 - RNA, 21 nt, mouse
gaacggcgucaugcaggaguu

SEQ ID NO: 400 - RNA, 23 nt, mouse
ugagcgccucggcgacagagccg

SEQ ID NO: 401 - RNA, 22 nt, mouse
ccugaacuaggggucuggagac

SEQ ID NO: 402 - RNA, 22 nt, mouse
acugcagugugagcacuucuag

SEQ ID NO: 403 - RNA, 22 nt, mouse
ggcugcagcgugaucgccugcu

SEQ ID NO: 404 - RNA, 21 nt, mouse
uguucagacugguguccauca

SEQ ID NO: 405 - RNA, 22 nt, mouse
uaacugcaacagcucucaguau

SEQ ID NO: 406 - RNA, 22 nt, mouse
uagugguuuacaaaguaauuca

SEQ ID NO: 407 - RNA, 19 nt, mouse
acuugaggggcaugaggau

SEQ ID NO: 408 - RNA, 22 nt, mouse
auacauacacgcacacauaaga

SEQ ID NO: 409 - RNA, 22 nt, mouse
uaagugcgugcauguauaugug

SEQ ID NO: 410 - RNA, 23 nt, mouse
ugaagguccuacugugugccagg

SEQ ID NO: 411 - RNA, 22 nt, mouse
uacuccagaauguggcaaucau

SEQ ID NO: 412 - RNA, 22 nt, mouse
ugguaagcugcagaacaugugu

SEQ ID NO: 413 - RNA, 20 nt, mouse
aggcaguguuguuagcuggc

SEQ ID NO: 414 - RNA, 21 nt, mouse
uaugcauauacacgcaugcaa

SEQ ID NO: 415 - RNA, 22 nt, mouse
gagugcuggaauuaaaggcaug

SEQ ID NO: 416 - RNA, 23 nt, mouse
uauguguguguguauguguguaa

SEQ ID NO: 417 - RNA, 24 nt, mouse
augcauggguguauaguugagugc

SEQ ID NO: 418 - RNA, 23 nt, mouse
ugaguucgaggccagccugcuca

SEQ ID NO: 419 - RNA, 22 nt, mouse
uauguguuccuggcuggcuugg

SEQ ID NO: 420 - RNA, 22 nt, mouse
cuuuggauggagaaagaggggg

SEQ ID NO: 421 - RNA, 22 nt, mouse
caccagucccaccacgcgguag

SEQ ID NO: 422 - RNA, 22 nt, mouse
gagcagcagaggaucuggaggu

SEQ ID NO: 423 - RNA, 22 nt, mouse
gcaagggagagggugaagggag

SEQ ID NO: 424 - RNA, 24 nt, mouse
agucaugguguucggucuuaguuu

SEQ ID NO: 425 - RNA, 22 nt, mouse
aggacgagcuagcugagugcug

SEQ ID NO: 426 - RNA, 22 nt, mouse
uuuaggcagagcacucguacag

SEQ ID NO: 427 - RNA, 22 nt, mouse
ccagugcuguuagaagagggcu

SEQ ID NO: 428 - RNA, 26 nt, mouse
gccgggcagugguggcacaugcuuuu

SEQ ID NO: 429 - RNA, 23 nt, mouse
ugugucacuggggauaggcuuug

SEQ ID NO: 430 - RNA, 18 nt, mouse
guaaaggcugggcugaga

SEQ ID NO: 431 - RNA, 20 nt, mouse
uugggaggguccuggggagg

SEQ ID NO: 432 - RNA, 24 nt, mouse
agcugcgcugcuccugguaacugc

SEQ ID NO: 433 - RNA, 25 nt, mouse
aggagggaggggaugggccaaguuc

SEQ ID NO: 434 - RNA, 22 nt, mouse
uaggcuagagagagguugggga

SEQ ID NO: 435 - RNA, 21 nt, mouse
uggcucauuuagaagcagcca

SEQ ID NO: 436 - RNA, 22 nt, mouse
gcugccuauacaugggcuuucc

SEQ ID NO: 437 - RNA, 24 nt, mouse
auuggagcugagauucugcgggau

SEQ ID NO: 438 - RNA, 22 nt, mouse
cuaccuuugauaguccacugcc

SEQ ID NO: 439 - RNA, 22 nt, mouse
ugaggaauccugaucucucgcc

SEQ ID NO: 440 - RNA, 24 nt, mouse
uuggcagucaagauauuguuuagc

SEQ ID NO: 441 - RNA, 22 nt, mouse
guggucacaguuggcgccagcc

SEQ ID NO: 442 - RNA, 23 nt, mouse
uuugaucugaugagcuaagcugg

SEQ ID NO: 443 - RNA, 22 nt, mouse
cacauggcacucaacucugcag

SEQ ID NO: 444 - RNA, 22 nt, mouse
guugaagguuaauuagcagagu

SEQ ID NO: 445 - RNA, 22 nt, mouse
uacauacacacauacacacgca

SEQ ID NO: 446 - RNA, 22 nt, mouse
gugugugcguacauguacaugu

SEQ ID NO: 447 - RNA, 22 nt, mouse
uauacaugagagcauacauaga

SEQ ID NO: 448 - RNA, 22 nt, mouse
aggggaaaaugccuuucuccca

SEQ ID NO: 449 - RNA, 21 nt, mouse
gaauggggcuguuuccccucc

SEQ ID NO: 450 - RNA, 18 nt, mouse
uggccaaggaugagaacu

SEQ ID NO: 451 - RNA, 23 nt, mouse
cacaggugggaagugugugucca

SEQ ID NO: 452 - RNA, 22 nt, mouse
cucagaccuuucuaccugucag

SEQ ID NO: 453 - RNA, 22 nt, mouse
gugaguggccaggguggggcug

SEQ ID NO: 454 - RNA, 21 nt, mouse
gguggugcaggcaggagagcc

SEQ ID NO: 455 - RNA, 23 nt, mouse
gucucuaaagcuagacguuccgg

SEQ ID NO: 456 - RNA, 24 nt, mouse
aauggaugcgaugguucccaugcu

SEQ ID NO: 457 - RNA, 20 nt, mouse
auaauacaaccugcuaagug

SEQ ID NO: 458 - RNA, 22 nt, mouse
cacagaacaugcagugagaacu

SEQ ID NO: 459 - RNA, 22 nt, mouse
ugagauccaacuguaaggcauu

SEQ ID NO: 460 - RNA, 22 nt, mouse
ugggauuaaaggcaugcaccac

SEQ ID NO: 461 - RNA, 22 nt, mouse
cccugggaggagacguggauuc

SEQ ID NO: 462 - RNA, 23 nt, mouse
acggguauucuuggguggauaau

SEQ ID NO: 463 - RNA, 22 nt, mouse
ccugugaaauucaguucuucag

SEQ ID NO: 464 - RNA, 17 nt, mouse
uuugugacguugcagcu

SEQ ID NO: 465 - RNA, 22 nt, mouse
caacgacaucaaaccaccugau

SEQ ID NO: 466 - RNA, 23 nt, mouse
acauacuucuuuauguacccaua

SEQ ID NO: 467 - RNA, 22 nt, mouse
aggcggagacuugggcaauugc

SEQ ID NO: 468 - RNA, 25 nt, mouse
cugguuucacaugguggcuuagauu

SEQ ID NO: 469 - RNA, 23 nt, mouse
agguugggauuugucgcaaugcu

SEQ ID NO: 470 - RNA, 19 nt, mouse
cucacugaacaaugaaugc

SEQ ID NO: 471 - RNA, 22 nt, mouse
gcuggucaaacggaaccaaguc

SEQ ID NO: 472 - RNA, 22 nt, mouse
gaacauccugcauagugcugcc

SEQ ID NO: 473 - RNA, 22 nt, mouse
ugaaaggugccauacuauguau

SEQ ID NO: 474 - RNA, 22 nt, mouse
uggcgaacacagaauccauacu

SEQ ID NO: 475 - RNA, 21 nt, mouse
acuguugcuaacaugcaacuc

SEQ ID NO: 476 - RNA, 21 nt, mouse
ugguuaucccuguccucuucg

SEQ ID NO: 477 - RNA, 22 nt, mouse
uacacacacacacacaaguaaa

SEQ ID NO: 478 - RNA, 23 nt, mouse
gaacaucacagcaagucugugcu

SEQ ID NO: 479 - RNA, 20 nt, mouse
uaucuauuaaagaggcuagc

SEQ ID NO: 480 - RNA, 21 nt, mouse
cccaccgggggaugaauguca

SEQ ID NO: 481 - RNA, 19 nt, mouse
uacgcacgcacacacacac

SEQ ID NO: 482 - RNA, 19 nt, mouse
uuguacaugguaggcuuuc

SEQ ID NO: 483 - RNA, 21 nt, mouse
uucacuggaguuuguuucagu

SEQ ID NO: 484 - RNA, 21 nt, mouse
ugaauauacacacacuuacac

SEQ ID NO: 485 - RNA, 21 nt, mouse
ugcggccggugcucagucggc

SEQ ID NO: 486 - RNA, 22 nt, mouse
gcacugagcuagcucucccucc

SEQ ID NO: 487 - RNA, 21 nt, mouse
gagcauugcaugcugggacau

SEQ ID NO: 488 - RNA, 21 nt, mouse
cuggcuggggaaaaugacugg

SEQ ID NO: 489 - RNA, 20 nt, mouse
auaagguagaaagcacuaaa

SEQ ID NO: 490 - RNA, 20 nt, mouse
gggcuggagagauggcucag

SEQ ID NO: 491 - RNA, 22 nt, mouse
ucucuccagcccccauaauaag

SEQ ID NO: 492 - RNA, 23 nt, mouse
uguugcggaccaggggaauccga

SEQ ID NO: 493 - RNA, 19 nt, mouse
aagguuaggccagccuggu

SEQ ID NO: 494 - RNA, 23 nt, mouse
uuuggggcuguggugccaccagc

SEQ ID NO: 495 - RNA, 21 nt, mouse
agcuugugaugagacaucucc

SEQ ID NO: 496 - RNA, 22 nt, mouse
acuccugcaugacgccguuccc

SEQ ID NO: 497 - RNA, 22 nt, mouse
ugucucagaaaacaaccaagga

SEQ ID NO: 498 - RNA, 23 nt, mouse
ugcccugucuguucugcccacag

SEQ ID NO: 499 - RNA, 22 nt, mouse
aacuugugaugaggugagacag

SEQ ID NO: 500 - RNA, 21 nt, mouse
caggcggccucagcucucacu

SEQ ID NO: 501 - RNA, 22 nt, mouse
auggucacaguggacaucaacc

SEQ ID NO: 502 - RNA, 22 nt, mouse
ucuugggacauaguguaaggca

SEQ ID NO: 503 - RNA, 22 nt, mouse
cagcucauggagaccuaggugg

SEQ ID NO: 504 - RNA, 24 nt, mouse
aguugugugugcauguucaugucu

SEQ ID NO: 505 - RNA, 22 nt, mouse
ugauguguguguacauguacau

SEQ ID NO: 506 - RNA, 20 nt, mouse
caggcuucuggcuauauucc

SEQ ID NO: 507 - RNA, 23 nt, mouse
uuuggcacuagcacauuuuugcu

SEQ ID NO: 508 - RNA, 23 nt, mouse
uaauacugccggguaaugaugga

SEQ ID NO: 509 - RNA, 22 nt, mouse
uaaucucagcuggcaacuguga

SEQ ID NO: 510 - RNA, 22 nt, mouse
gcagcagggugaaacugacaca

SEQ ID NO: 511 - RNA, 22 nt, mouse
acugcccuaagugcuccuucug

SEQ ID NO: 512 - RNA, 23 nt, mouse
uguguguguacauguacauguga

SEQ ID NO: 513 - RNA, 22 nt, mouse
auaagugugugcauguauaugu

SEQ ID NO: 514 - RNA, 25 nt, mouse
agucccaggaugcacugcagcuuuu

SEQ ID NO: 515 - RNA, 26 nt, mouse
uggguuaaaggauuuaccugaggcca

SEQ ID NO: 516 - RNA, 22 nt, mouse
auaaagcuagauaaccgaaagu

SEQ ID NO: 517 - RNA, 22 nt, mouse
acuccauuuguuuugaugaugg

SEQ ID NO: 518 - RNA, 23 nt, mouse
uuaaugcuaauugugauaggggu

SEQ ID NO: 519 - RNA, 22 nt, mouse
aacuggccuacaaagucccagu

SEQ ID NO: 520 - RNA, 22 nt, mouse
uucccuuugucauccuaugccu

SEQ ID NO: 521 - RNA, 21 nt, mouse
ugagaugaagcacuguagcuc

SEQ ID NO: 522 - RNA, 22 nt, mouse
ugagguaguagguuguaugguu

SEQ ID NO: 523 - RNA, 22 nt, mouse
ugagguaggagguuguauaguu

SEQ ID NO: 524 - RNA, 22 nt, mouse
uagcaccaucugaaaucgguua

SEQ ID NO: 525 - RNA, 22 nt, mouse
uggcagugucuuagcugguugu

SEQ ID NO: 526 - RNA, 22 nt, mouse
aaaagcuggguugagagggcga

SEQ ID NO: 527 - RNA, 21 nt, mouse
ugguagacuauggaacguagg

SEQ ID NO: 528 - RNA, 22 nt, mouse
uagguaguuuccuguuguuggg

SEQ ID NO: 529 - RNA, 22 nt, mouse
ucaggcucaguccccucccgau

SEQ ID NO: 530 - RNA, 16 nt, mouse
augguggcacggaguc

SEQ ID NO: 531 - RNA, 21 nt, mouse
cccagauaauagcacucucaa

SEQ ID NO: 532 - RNA, 18 nt, mouse
gcgugugcuugcuguggg

SEQ ID NO: 533 - RNA, 18 nt, mouse
aucucgcuggggccucca

SEQ ID NO: 534 - RNA, 21 nt, mouse
aacauccugguccuguggaga

SEQ ID NO: 535 - RNA, 19 nt, mouse
ugcacugaaggcacacagc

SEQ ID NO: 536 - RNA, 22 nt, mouse
aauccuuugucccugggugaaa

SEQ ID NO: 537 - RNA, 22 nt, mouse
agcgggcacagcugugagagcc

SEQ ID NO: 538 - RNA, 19 nt, mouse
ggugcucacauguccuccu

SEQ ID NO: 539 - RNA, 22 nt, mouse
ugugaguuguuccucaccugga

SEQ ID NO: 540 - RNA, 22 nt, mouse
auuccuggaaauacuguucuug

SEQ ID NO: 541 - RNA, 22 nt, mouse
acucaaaauggaggcccuaucu

SEQ ID NO: 542 - RNA, 22 nt, mouse
uaugugggacgguaaaccgcuu

SEQ ID NO: 543 - RNA, 22 nt, mouse
acuuaaacgugguuguacuugc

SEQ ID NO: 544 - RNA, 22 nt, mouse
ucucugggccugugucuuaggc

SEQ ID NO: 545 - RNA, 22 nt, mouse
uuauaaagcaaugagacugauu

SEQ ID NO: 546 - RNA, 22 nt, mouse
uggagacgcggcccuguuggag

SEQ ID NO: 547 - RNA, 22 nt, mouse
aacacaccuguucaaggauuca

SEQ ID NO: 548 - RNA, 23 nt, mouse
agguuacccgagcaacuuugcau

SEQ ID NO: 549 - RNA, 21 nt, mouse
uccgguucucagggcuccacc

SEQ ID NO: 550 - RNA, 22 nt, mouse
aacugugucuuuucugaauaga

SEQ ID NO: 551 - RNA, 22 nt, mouse
auguaugugugcauguacaugu

SEQ ID NO: 552 - RNA, 22 nt, mouse
ugugugcaugugcuugugugua

SEQ ID NO: 553 - RNA, 22 nt, mouse
uaagugcgcgcauguauaugcg

SEQ ID NO: 554 - RNA, 20 nt, mouse
auguauaaauguauacacac

SEQ ID NO: 555 - RNA, 22 nt, mouse
ugaacuauugcaguagccuccu

SEQ ID NO: 556 - RNA, 22 nt, mouse
acuugugugugcauguauaugu

SEQ ID NO: 557 - RNA, 22 nt, mouse
ugucuugugugugcauguucau

SEQ ID NO: 558 - RNA, 21 nt, mouse
uaggacacauggucuacuucu

SEQ ID NO: 559 - RNA, 24 nt, mouse
agggagaugcugguacagaggcuu

SEQ ID NO: 560 - RNA, 22 nt, mouse
ugggaaaguucucaggcuucug

SEQ ID NO: 561 - RNA, 23 nt, mouse
ugggacgagaucaugaggccuuc

SEQ ID NO: 562 - RNA, 25 nt, mouse
aagggagcuggcucaggagagaguc

SEQ ID NO: 563 - RNA, 22 nt, mouse
acgcccuucccccccuucuuca

SEQ ID NO: 564 - RNA, 21 nt, mouse
uuccugucagccgugggugcc

SEQ ID NO: 565 - RNA, 24 nt, mouse
agucaggcugcuggcuauacacca

SEQ ID NO: 566 - RNA, 22 nt, mouse
ccauagcacagaagcacuccca

SEQ ID NO: 567 - RNA, 22 nt, mouse
cagugggccgugaaagguagcc

SEQ ID NO: 568 - RNA, 22 nt, mouse
ucaacaaaaucacugaugcugg

SEQ ID NO: 569 - RNA, 22 nt, mouse
guuccugcugaacugagccagu

SEQ ID NO: 570 - RNA, 22 nt, mouse
auauacauacacacccauauac

SEQ ID NO: 571 - RNA, 22 nt, mouse
uguggacaccgugggagguugg

SEQ ID NO: 572 - RNA, 21 nt, mouse
cugugacacacccgcucccag

SEQ ID NO: 573 - RNA, 21 nt, mouse
agucaggcuccuggcaggagu

SEQ ID NO: 574 - RNA, 21 nt, mouse
cucuacucccugccccagcca

SEQ ID NO: 575 - RNA, 23 nt, mouse
acgcucugcuuugcucccccaga

SEQ ID NO: 576 - RNA, 20 nt, mouse
cggggcagcucaguacagga

SEQ ID NO: 577 - RNA, 21 nt, mouse
acauagaaaaggcagucugca

SEQ ID NO: 578 - RNA, 22 nt, mouse
gcucuuuucacauugugcuacu

SEQ ID NO: 579 - RNA, 22 nt, mouse
aaccguggcuuucgauuguuac

SEQ ID NO: 580 - RNA, 22 nt, mouse
aggcuacaacacaggacccggg

SEQ ID NO: 581 - RNA, 21 nt, mouse
cuguacagccuccuagcuuuc

SEQ ID NO: 582 - RNA, 21 nt, mouse
ggucaagaggcgccugggaac

SEQ ID NO: 583 - RNA, 21 nt, mouse
ucugucauucuguaggccaau

SEQ ID NO: 584 - RNA, 23 nt, mouse
uggucgaccagcuggaaaguaau

SEQ ID NO: 585 - RNA, 22 nt, mouse
ucaucacguggugacgcaacau

SEQ ID NO: 586 - RNA, 23 nt, mouse
ccccucaggccaccagagcccgg

SEQ ID NO: 587 - RNA, 21 nt, mouse
caaaccacacugugguguuag

SEQ ID NO: 588 - RNA, 22 nt, mouse
uaaggcuccuuccugugcuugc

SEQ ID NO: 589 - RNA, 23 nt, mouse
ugacuggcaccauucuggauaau

SEQ ID NO: 590 - RNA, 22 nt, mouse
cggccccacgcaccaggguaag

SEQ ID NO: 591 - RNA, 22 nt, mouse
agggagagcagggcaggguuuc

SEQ ID NO: 592 - RNA, 25 nt, mouse
ugugcauguguguauaguugugugc

SEQ ID NO: 593 - RNA, 20 nt, mouse
ugugugugugugugugugug

SEQ ID NO: 594 - RNA, 20 nt, mouse
ugguagaccggugacguaca

SEQ ID NO: 595 - RNA, 21 nt, mouse
guuacauggugaagcccaguu

SEQ ID NO: 596 - RNA, 23 nt, mouse
aggucuguagcucaguuggcaga

SEQ ID NO: 597 - RNA, 22 nt, mouse
acuggagacggaagcugcaaga

SEQ ID NO: 598 - RNA, 21 nt, mouse
uuggcagaaagggcagcugug

SEQ ID NO: 599 - RNA, 23 nt, mouse
ucugggcagagcugcaggagaga

SEQ ID NO: 600 - RNA, 23 nt, mouse
guaagugagggcaagccuucugg

SEQ ID NO: 601 - RNA, 23 nt, mouse
aggagguccuggggccgcccuga

SEQ ID NO: 602 - RNA, 21 nt, mouse
ugggcccuccagaccucaugc

SEQ ID NO: 603 - RNA, 24 nt, mouse
uggcagcuggcaagucagaaugca

SEQ ID NO: 604 - RNA, 22 nt, mouse
cuggcgccaacugugaccacug

SEQ ID NO: 605 - RNA, 22 nt, mouse
ugagguaguagauuguauaguu

SEQ ID NO: 606 - RNA, 22 nt, mouse
gugccuacugagcugaaacagu

SEQ ID NO: 607 - RNA, 22 nt, mouse
uggcucaguucagcaggaacag

SEQ ID NO: 608 - RNA, 22 nt, mouse
gaggaacuagccuucucucagc

SEQ ID NO: 609 - RNA, 23 nt, mouse
uggaagacuugugauuuuguugu

SEQ ID NO: 610 - DNA, 29 bp, artificial
  primer
ttggatccagggcctgtatgtgtgaatga

SEQ ID NO: 611 - DNA, 32 bp, artificial
  primer
ttttgctagcacacttgtgctttagaagttgc

SEQ ID NO: 612 - DNA, 32 bp, artificial
  primer
ttggatccaaaattactcagcccatgtagttg

SEQ ID NO: 613 - DNA, 30 bp, artificial
  primer
ttttgctagcttagccagagaagtgcaacc

SEQ ID NO: 614 - DNA, 30 bp, artificial
  primer
ttggatccatgtgtcggagaagtggtcatc

SEQ ID NO: 615 - DNA, 32 bp, artificial
  primer
ttttgctagcctccaaaggaagagaggcagtt

SEQ ID NO: 616 - DNA, 23 bp, artificial
  primer
tgtaaacatcctcgactggaagc

SEQ ID NO: 617 - DNA, 22 bp, artificial
  primer
tgtaaacatcctacactcagct

SEQ ID NO: 618 - DNA, 23 bp, artificial
  primer
tgtaaacatcctacactctcagc

SEQ ID NO: 619 - DNA, 22 bp, artificial
  primer
ctttcagtcagatgtttgctgc

SEQ ID NO: 620 - DNA, 23 bp, artificial
  primer
tgtaaacatccttgactggaagc

SEQ ID NO: 621 - DNA, 17 bp, artificial
  primer
ctcgcttcggcagcaca

SEQ ID NO: 622 - DNA, 20 bp, artificial
  primer
aacgcttcacgaatttgcgt

(19) United States
(12) Patent Application Publication
(10) Pub. No.: US 2017/0044541 A1
(43) Pub. Date: Feb. 16, 2017

(54) miRNAs Enhancing Cell Productivity

(71) Applicant: Hochschule Biberach, Biberach, DE (US)

(72) Inventors: Kerstin OTTE, Ulm (DE); René HANDRICK, Biberach (DE); Simon FISCHER, Staig (DE)

(21) Appl. No.: 15/306,035

(22) PCT Filed: Apr. 24, 2015
(86) PCT No.: PCT/EP2015/058975

§ 371 Date: Oct. 21, 2016

(30) Foreign Application Priority Data
Apr. 25, 2014 (EP) 14166041.5

(51) Int. Cl. C12N 15/113

(52) U.S. Cl. CPC C12N 15/113; C12N 2310/113; C12N 2310/141

(57) ABSTRACT

The invention relates to a nucleic acid construct comprising at least two different regions, wherein the regions are selected of a first region encoding for at least one miRNA stimulating cellular production of a biomolecule in a cell, a second region encoding for at least one miRNA and/or miRNA-inhibitor suppressing cell death, and a third region encoding for at least one miRNA and/or miRNA-inhibitor regulating cell proliferation. The invention further relates to a cell comprising a respective nucleic acid construct and to a method for increasing the yield of a biomolecule produced by a cell cultured in vitro comprising at least two steps selected of stimulating cellular production of the biomolecule, reducing cell death, and regulating proliferation.

### FIELD OF THE INVENTION

The invention relates to a nucleic acid construct comprising at least two different regions each encoding for at least one miRNA or miRNA-inhibitor having distinct functions such as stimulating cellular production of a biomolecule, regulating cell survival and/or regulating proliferation. The invention further relates to a cell comprising such a nucleic acid construct. Moreover, the invention relates to a method for increasing the yield of a biomolecule produced by a cell cultured in vitro.

### BACKGROUND OF THE INVENTION

Since the first pharmaceuticals, such as insulin, were biotechnologically produced, i.e. in living cells, the field of biopharmaceutical manufacturing has grown enormously. Nowadays a large variety of pharmaceutical compounds is produced using biotechnological methods. Such compounds comprise antibodies, cytokines, hormones as well as anticoagulants. Whereas most compounds are produced in lower organisms such as bacteria and yeast, an increasing number of interesting pharmaceuticals, in particular large and complex proteins, need to be expressed in cells derived from higher order organisms such as mammals. This is due to the need of specific enzymes or other molecules involved in the synthesis of the desired compounds, which mostly developed late during evolution. For example, most post-translational modifications of proteins are mediated by enzymes, which are expressed by most mammalian cells but not in bacteria or yeast. Accordingly, biopharmaceuticals are nowadays extensively produced in mammalian cell factories. Due to the rapidly growing demand of biopharmaceuticals, in particular recombinant proteins, various strategies are pursued to achieve higher product titers while maintaining maximal product quality. However, moderate product titers and low stress tolerance in bioreactors are still considerable challenges compared to prokaryotic expression systems. Overcoming limitations of mammalian manufacturing cell lines has been addressed by different cell line engineering approaches to steadily increase production efficiency (e.g. Kramer et al., 2010). Apart from gene knockouts mediated e.g. by zinc finger nucleases, mega nucleases or more recently by using the CRISPR/Cas9 system, the introduction of beneficial genes such as Bcl-XL or AVEN was frequently applied to engineer mammalian cell factories. However, the vast majority of those techniques is complex, labour intensive or requires a substantial amount of time for establishment. Moreover, traditional cell engineering strategies usually rely on the overexpression of one or few secretion enhancing genes. The constitutive overexpression, however, adds additional translational burden to the production cell and, thus, lowers its capacity for producing the biopharmaceutical of interest.

Therefore, there is a need in the art to provide tools and methods to increase production efficiency in biopharmaceutical manufacturing, in particular to increase the productivity while maintaining or even minimizing the cells consumption of energy and nutrients not directed to the production of the compound of interest.

### SUMMARY OF THE INVENTION

In a first aspect, the invention relates to a nucleic acid construct comprising at least two different regions, wherein the regions are selected of a first region encoding for at least one miRNA stimulating cellular production of a biomolecule in a cell, the miRNA is selected from group 1, a second region encoding for at least one miRNA and/or miRNA-inhibitor suppressing cell death, the miRNA is selected from group 2 and the miRNA-inhibitor inhibits a miRNA selected from group 3, and a third region encoding for at least one miRNA and/or miRNA-inhibitor regulating cell proliferation, the miRNA is selected from group 4 or 5 and the miRNA-inhibitor inhibits a miRNA selected from group 4 or 5, wherein group 1 consists of SEQ ID NO.: 1, 2, 3, 4, 6, 8, 9, 10, 11, 12, 13, 14, 15, 16, 17, 18, 19, 20, 21, 25, 26, 27, 28, 29, 32, 34, 37, 39, 40, 41, 42, 44, 45, 46, 47, 48, 49, 51, 52, 53, 55, 56, 57, 58, 62, 63, 66, 67, 69, 70, 75, 76, 77, 80, 81, 82, 83, 86, 88, 90, 91, 93, 98, 99, 100, 101, 102, 103, 104, 106, 109, 110, 111, 112, 113, 114, 115, 116, 117, 118, 120, 121, 128, 130, 132, 134, 136, 137, 138, 141, 142, 143, 147, 151, 152, 155, 158, 163, 165, 171, 174, 182, 183, 185, 186, 188, 190, 201, 203, 207, 211, 212, 214, 217, 222, 223, 228, 230, 233, 238, 239, 240, 254, 255, 269, 276, 278, 279, 285, and 294; group 2 consists of SEQ ID NO.: 1, 2, 3, 4, 6, 8, 9, 20, 52, 98, 5, 7, 24, 31, 33, 50, 54, 59, 60, 61, 64, 68, 71, 73, 74, 79, 85, 87, 89, 92, 94, 95 97, 145, 153, 154, 156, 159, 161, 166, 167, 168, 169, 170, 172, 175, 176, 178, 179, 180, 181, 184, 191, 192, 194, 195, 196, 197, 199, 200, 204, 205, 206, 208, 209, 210, 213, 216, 219, 220, 221, 224, 225, 226, 227, 229, 231, 236, 237, 241, 242, 243, 245, 247, 248, 251, 252, 253, 256, 257, 258, 259, 260, 262, 265, 266, 268, 271, 272, 273, 274, 277, 280, 282, 284, 289, 293, and 295; group 3 consists of SEQ ID NO.: 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 605, 607, 608, and 609; group 4 consists of SEQ ID NO.: 5, 7, 68, 79, 94, 10, 11, 12, 13, 14, 15, 16, 17, 18, 19, 22, 23, 30, 35, 36, 38, 43, 65, 72, 78, 84, 96, 105, 107, 108, 119, 122, 123, 124, 125, 126, 127, 129, 131, 133, 135, 139, 140, 144, 146, 148, 149, 150, 160, 162, 164, 173, 177, 187, 189, 193, 198, 202, 215, 218, 232, 234, 235, 244, 246, 249, 250, 261, 263, 264, 267, 270, 275, 281, 283, 287, 288, and 291; and group 5 consists of SEQ ID NO.: 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, and 606.

In a further aspect, the invention relates to a cell comprising the nucleic acid construct of the invention.

In a further aspect, the invention relates to a method for increasing the yield of a biomolecule produced by a cell cultured in vitro comprising at least two steps selected of stimulating cellular production of the biomolecule by increasing the level of at least one miRNA selected from group 1 in the cell, reducing cell death by increasing the level of at least one miRNA selected from group 2 in the cell and/or decreasing the level of at least one miRNA selected from group 3, and regulating proliferation of the cell by increasing the level of at least one miRNA selected from group 4 or 5 in the cell and/or decreasing the level of at least one miRNA selected from group 4 or 5, wherein group 1 consists of SEQ ID NO.: 1, 2, 3, 4, 6, 8, 9, 10, 11, 12, 13, 14, 15, 16, 17, 18, 19, 20, 21, 25, 26, 27, 28, 29, 32, 34, 37, 39, 40, 41, 42, 44, 45, 46, 47, 48, 49, 51, 52, 53, 55, 56, 57, 58, 62, 63, 66, 67, 69, 70, 75, 76, 77, 80, 81, 82, 83, 86, 88, 90, 91, 93, 98, 99, 100, 101, 102, 103, 104, 106, 109, 110, 111, 112, 113, 114, 115, 116, 117, 118, 120, 121, 128, 130, 132, 134, 136, 137, 138, 141, 142, 143, 147, 151, 152, 155, 158, 163, 165, 171, 174, 182, 183, 185, 186, 188, 190, 201, 203, 207, 211, 212, 214, 217, 222, 223, 228, 230, 233, 238, 239, 240, 254, 255, 269, 276, 278, 279, 285, and 294; group 2 consists of SEQ ID NO.: 1, 2, 3, 4, 6, 8, 9, 20, 52, 98, 5, 7, 24, 31, 33, 50, 54, 59, 60, 61, 64, 68, 71, 73, 74, 79, 85, 87, 89, 92, 94, 95 97, 145, 153, 154, 156, 159, 161, 166, 167, 168, 169, 170, 172, 175, 176, 178, 179, 180, 181, 184, 191, 192, 194, 195, 196, 197, 199, 200, 204, 205, 206, 208, 209, 210, 213, 216, 219, 220, 221, 224, 225, 226, 227, 229, 231, 236, 237, 241, 242, 243, 245, 247, 248, 251, 252, 253, 256, 257, 258, 259, 260, 262, 265, 266, 268, 271, 272, 273, 274, 277, 280, 282, 284, 289, 293, and 295; group 3 consists of SEQ ID NO.: 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 605, 607, 608, and 609; group 4 consists of SEQ ID NO.: 5, 7, 68, 79, 94, 10, 11, 12, 13, 14, 15, 16, 17, 18, 19, 22, 23, 30, 35, 36, 38, 43, 65, 72, 78, 84, 96, 105, 107, 108, 119, 122, 123, 124, 125, 126, 127, 129, 131, 133, 135, 139, 140, 144, 146, 148, 149, 150, 160, 162, 164, 173, 177, 187, 189, 193, 198, 202, 215, 218, 232, 234, 235, 244, 246, 249, 250, 261, 263, 264, 267, 270, 275, 281, 283, 287, 288, and 291; and group 5 consists of SEQ ID NO.: 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, and 606.

In a further aspect, the invention relates to a method for producing a biomolecule in a cell comprising the steps propagating the cell in a cell culture, increasing the level of at least one miRNA selected from the group consisting of SEQ ID NO.: 1, 2, 3, 4, 5, 6, 7, 8, 9, 10, 11, 12, 13, 14, 15, 16, 17, 18, 19, and 20, and isolating the biomolecule from the cell culture.

In a further aspect, the invention relates to the use of a combination of at least one miRNA selected from group 1, at least one miRNA selected from group 2 and/or at least one miRNA-inhibitor inhibiting a miRNA selected from group 3, and at least one miRNA selected from group 4 or 5 and/or at least one miRNA-inhibitor inhibiting a miRNA selected from group 4 or 5, in producing a biomolecule in a cell, wherein group 1 consists of SEQ ID NO.: 1, 2, 3, 4, 6, 8, 9, 10, 11, 12, 13, 14, 15, 16, 17, 18, 19, 20, 21, 25, 26, 27, 28, 29, 32, 34, 37, 39, 40, 41, 42, 44, 45, 46, 47, 48, 49, 51, 52, 53, 55, 56, 57, 58, 62, 63, 66, 67, 69, 70, 75, 76, 77, 80, 81, 82, 83, 86, 88, 90, 91, 93, 98, 99, 100, 101, 102, 103, 104, 106, 109, 110, 111, 112, 113, 114, 115, 116, 117, 118, 120, 121, 128, 130, 132, 134, 136, 137, 138, 141, 142, 143, 147, 151, 152, 155, 158, 163, 165, 171, 174, 182, 183, 185, 186, 188, 190, 201, 203, 207, 211, 212, 214, 217, 222, 223, 228, 230, 233, 238, 239, 240, 254, 255, 269, 276, 278, 279, 285, and 294; group 2 consists of SEQ ID NO.: 1, 2, 3, 4, 6, 8, 9, 20, 52, 98, 5, 7, 24, 31, 33, 50, 54, 59, 60, 61, 64, 68, 71, 73, 74, 79, 85, 87, 89, 92, 94, 95 97, 145, 153, 154, 156, 159, 161, 166, 167, 168, 169, 170, 172, 175, 176, 178, 179, 180, 181, 184, 191, 192, 194, 195, 196, 197, 199, 200, 204, 205, 206, 208, 209, 210, 213, 216, 219, 220, 221, 224, 225, 226, 227, 229, 231, 236, 237, 241, 242, 243, 245, 247, 248, 251, 252, 253, 256, 257, 258, 259, 260, 262, 265, 266, 268, 271, 272, 273, 274, 277, 280, 282, 284, 289, 293, and 295; group 3 consists of SEQ ID NO.: 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 605, 607, 608, and 609; group 4 consists of SEQ ID NO.: 5, 7, 68, 79, 94, 10, 11, 12, 13, 14, 15, 16, 17, 18, 19, 22, 23, 30, 35, 36, 38, 43, 65, 72, 78, 84, 96, 105, 107, 108, 119, 122, 123, 124, 125, 126, 127, 129, 131, 133, 135, 139, 140, 144, 146, 148, 149, 150, 160, 162, 164, 173, 177, 187, 189, 193, 198, 202, 215, 218, 232, 234, 235, 244, 246, 249, 250, 261, 263, 264, 267, 270, 275, 281, 283, 287, 288, and 291; and group 5 consists of SEQ ID NO.: 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, and 606.

### BRIEF DESCRIPTION OF THE DRAWINGS

FIG. 1 shows the normalized specific SEAP productivity of CHO-SEAP control cells transfected with a functional anti-SEAP control siRNA for all 73 screen plates. Each column represents the mean value of the indicated screen plate. Data was normalized to the mean value of the respective non-targeting control miRNA. Error bars indicate the standard deviation (SD) of three independent transfections.

FIG. 2 shows an overview on the numbers of miRNAs mimics from the primary screen in CHO-SEAP cells, which induced significant changes (p<0.05), as percentage of the miRNA library. Cake charts are given for each considered bioprocess relevant parameter.

FIG. 3 shows that the entire miR-30 family contributes to enhanced culture performance of CHO-SEAP cells. (A) Normalized volumetric SEAP productivity for all miR-30 miRNAs exhibiting increased SEAP productivity in the primary miRNA screen (A) and in the secondary (validation) miRNA screen (B). Normalized viable cell density for all miR-30 miRNAs exhibiting increased SEAP under agitated culture conditions (C). Influence of miR-30 miRNAs on apoptosis and necrosis under agitated culture conditions. Error bars represent the SD of three independent transfections. For statistical analysis an unpaired two-tailed t-test was applied (** p<0.01; *** p<0.001). Normalized increase in volumetric (y-axis) against specific SEAP productivity (x-axis) shown for miRNAs significantly influencing both parameters. (E) Normalized decrease in apoptosis (y-axis) against increase in specific SEAP productivity (x-axis) shown for miRNAs significantly influencing both parameters (F). Respective miR-30 family members are indicated.

FIG. 4 shows the results of a scale-up transfection of miR-30 family members for screen validation in CHO-SEAP cells. Influence on normalized volumetric SEAP productivity (A) and viable cell density (VCD) and viability (B) following introduction of either single miR-30a-5p and miR-30c-5p mimics or combinations of both miRNAs after 72 h following transient introduction. Values were normalized to the miR-NT control and error bars represent the SD of three independent transfections. For statistical analysis an unpaired two-tailed t-test was applied (* p<0.05; ** p<0.01; *** p<0.001).

FIG. 5 shows a characterization of stable miR-30 overexpressing CHO-SEAP cell pools. miRNA overexpression in stable cell pools (A). Mature miR-30 levels are expressed relative to U6 snoRNA. miRNA overexpression is presented as fold-change value relative to endogenous miRNA level represented by the pEGP-MIR-Null control pool. Determination of volumetric SEAP productivity (B), viable cell density/viability (C), and specific SEAP productivity (D) during batch cultivation of MIR30a, MIR30c and MIR30e overexpressing cell pools compared to negative control and parental CHO-SEAP cells. Error bars represent the SD of three replicates. Statistical analysis: unpaired two-tailed t-test comparing each miR-30 overexpressing pool with the parental CHO-SEAP cell line (* p<0.05; ** p<0.01; *** p<0.001).

FIG. 6 shows (A) an analysis of endogenous miR-30a-5p (diamond) and miR-30c-5p (triangle) expression level during batch cultivation of CHO-SEAP cells. Analysis of miRNA expression level and viable cell density (dotted line) was performed at indicated days post seeding and changes in miRNA expression were calculated relative to the level at 48 h. (B) Analysis of apoptosis in CHO-SEAP cells after miR-30c-5p mimics/antagomiR (anti-miR-30c-5p) transfection by means of Nicoletti staining. DNA content of transfected cells was determined using flow cytometry and cells exhibiting DNA content less than 2n (Sub-G1/0) were quantified as percentage of the whole cell population. Error bars represent the SD of three independent transfections. (C) Relative mature miR-30c-5p abundance in CHO-SEAP cells after miR-30c-5p mimics/antagomiR (anti-miR-30c-5p) transfection. miRNA expression is presented as fold-change value relative to endogenous miRNA level represented by miR-NT transfected control cells (black column). Total RNA was isolated 72 h post transfection and RNA samples of triplicate transfections were pooled for reverse transcription. Mature miR-30c-5p levels are expressed relative to U6 snoRNA and error bars represent the SD of three technical replicates. Normalized (D) specific SEAP productivity and (E) viable cell density of CHO-SEAP cells 72 h following miR-30c-5p mimics/antagomiR (anti-miR-30c-5p) introduction. Data was normalized to values of the miR-NT transfected control cells (black column) and error bars represent the SD of three independent transfections. For statistical analysis an unpaired two-tailed t-test was applied (** p<0.01; *** p<0.001).

FIGS. 7 to 9 show the results of the secondary (validation) miRNA screen for regarding specific SEAP productivity (7), volumetric SEAP productivity (8) and proliferation (9). Data was normalized to values of the miR-NT transfected control cells and error bars represent the SD of three independent transfections. For statistical analysis an unpaired two-tailed t-test was applied (** p<0.01; *** p<0.001).

FIG. 10 shows activity of three apoptosis promoting miRNAs, miR-134-5p, miR-378-5p and let-7d-3p, in different human cell lines, namely SKOV 3, T98G, HCT 116 and SGBS (n=3+/−SD; +p<0.05, ** p<0.01, ***p<0.001 to non targeting control miR-NT) FIG. 11 shows the increased production of recombinant adeno-associated vectors (rAAVs) in HeLa cells upon transfection of miR-483.

### DETAILED DESCRIPTION OF THE INVENTION

In a first aspect, the invention relates to a nucleic acid construct comprising at least two different regions, wherein the regions are selected of a first region encoding for at least one miRNA stimulating cellular production of a biomolecule in a cell, the miRNA is selected from group 1, a second region encoding for at least one miRNA and/or miRNA-inhibitor suppressing cell death, the miRNA is selected from group 2 and the miRNA-inhibitor inhibits a miRNA selected from group 3, and a third region encoding for at least one miRNA and/or miRNA-inhibitor regulating cell proliferation, the miRNA is selected from group 4 or 5 and the miRNA-inhibitor inhibits a miRNA selected from group 4 or 5.

Micro RNAs (miRNAs) are endogenous small non-coding RNA molecules of about 22 nucleotides that post-transcriptionally regulate global gene expression in eukaryotic cells and are highly conserved across species. A single miRNA usually regulates up to hundreds of different messenger RNAs (mRNAs) and most mRNAs are expected to be targeted by multiple miRNAs. miRNA genes are transcribed by RNA polymerase-II and subsequently processed, giving rise to single-stranded mature miRNAs, which are incorporated into the RNA-induced silencing complex (RISC). As a central part of the RISC complex, the miRNA guides RISC to its mRNA targets, where the miRNA binds the 3′-untranslated region (3′UTR) of the mRNA transcript by partial complementary base pairing. Gene silencing occurs either through argonaute-2 (AGO2)-mediated mRNA cleavage or translational repression facilitated by AGO1 to 4, with both ways finally reducing the levels of corresponding proteins (van Rooij, 2011). In contrast to small interference RNAs (siRNAs), miRNAs are only partially complementary to binding sites within the 3′UTR of the target transcript, leading to less specificity and, thus, increasing the pool of potential target genes. Complete Watson-Crick base pairing is only compository at the miRNA “seed” region which covers the sequence between nucleotide 2 to 7/8 at the 5-end of the mature miRNA. miRNAs with identical “seed” sequences such as the miR-30 members are grouped into families. According to bioinformatic target prediction tools, members of the same family are supposed to share a large number of target mRNAs.

The inventors found that despite the large number of targets of a single miRNA, several miRNAs show a specific effect on certain cellular processes. By performing a functional high-content miRNA screen, using an entire murine miRNA mimics library comprising 1139 miRNAs, in a recombinant CHO-SEAP suspension cell line, the inventors revealed distinct miRNAs, which are suitable to improve specific cell functions. In particular, the miRNAs of group 1 (table 1) were found to stimulate cellular production of the biomolecule. The term “cellular production of a biomolecule” as used herein refers to the amount of a biomolecule produced per cell. Cellular production is also referred to as “specific productivity”, in contrast to the “volumetric productivity” of an entire culture, which refers to the yield of biomolecule that can be harvested from the culture. The level of cellular production mainly depends on the amount of biomolecule synthesized per time by one cell e.g. on protein translation speed, and where applicable on the efficiency, with which the biomolecule is secreted from the cell. Thus, miRNAs of group 1 are expected to influence one or even both processes. Within group 1, the miRNAs of group 9, consisting of SEQ ID NO.: 1, 3, 6, 8, 10, 29, 39, 40, 47, 49, 51, 55, 91, 103, 115, 132, 137, 171, 211 and 294 were found to show the most prominent effect on cellular production. Accordingly, the first region preferably encodes for at least one miRNA selected from group 9.

TABLE 1
miRNAs stimulating cellular production (group 1)

| SEQ ID NO.: | miRNA |
|---|---|
| 1 | mmu-miR-99b-3p |
| 2 | mmu-miR-767 |
| 3 | mmu-miR-30a-5p |
| 4 | mmu-miR-3062-5p |
| 6 | mmu-miR-200a-5p |
| 8 | mmu-miR-135a-1-3p |
| 9 | mmu-miR-743a-5p |
| 10 | mmu-miR-694 |
| 11 | mmu-miR-674-3p |
| 12 | mmu-miR-669d-3p |
| 13 | mmu-miR-301b-5p |
| 14 | mmu-miR-212-5p |
| 15 | mmu-miR-203-5p |
| 16 | mmu-miR-200b-5p |
| 17 | mmu-miR-200a-3p |
| 18 | mmu-miR-1968-5p |
| 19 | mmu-miR-150-3p |
| 20 | mmu-miR-30d-5p |
| 21 | mmu-miR-92b-5p |
| 25 | mmu-miR-871-3p |
| 26 | mmu-miR-760-5p |
| 27 | mmu-miR-741-3p |
| 28 | mmu-miR-713 |
| 29 | mmu-miR-700-5p |
| 32 | mmu-miR-669d-2-3p |
| 34 | mmu-miR-666-5p |
| 37 | mmu-miR-5623-5p |
| 39 | mmu-miR-5134 |
| 40 | mmu-miR-5132 |
| 41 | mmu-miR-5127 |
| 42 | mmu-miR-5124 |
| 44 | mmu-miR-5117-3p |
| 45 | mmu-miR-5111-3p |
| 46 | mmu-miR-5099 |
| 47 | mmu-miR-504-5p |
| 48 | mmu-miR-497-3p |
| 49 | mmu-miR-484 |
| 51 | mmu-miR-466f-3p |
| 52 | mmu-miR-463-5p |
| 53 | mmu-miR-3971 |
| 55 | mmu-miR-370-3p |
| 56 | mmu-miR-344g-5p |
| 57 | mmu-miR-344d-3p |
| 58 | mmu-miR-341-5p |
| 62 | mmu-miR-3113-3p |
| 63 | mmu-miR-3107-3p |
| 66 | mmu-miR-3094-5p |
| 67 | mmu-miR-3083-5p |
| 69 | mmu-miR-3074-5p |
| 70 | mmu-miR-3065-3p |
| 75 | mmu-miR-218-1-3p |
| 76 | mmu-miR-215-3p |
| 77 | mmu-miR-20b-5p |
| 80 | mmu-miR-1 b-3p |
| 81 | mmu-miR-1956 |
| 82 | mmu-miR-1953 |
| 83 | mmu-miR-193b-3p |
| 86 | mmu-miR-1898 |
| 88 | mmu-miR-155-5p |
| 90 | mmu-miR-149-5p |
| 91 | mmu-miR-143-5p |
| 93 | mmu-miR-136-5p |
| 98 | mmu-let-7a-1-3p |
| 99 | mmu-miR-875-5p |
| 100 | mmu-miR-802-5p |
| 101 | mmu-miR-708-3p |
| 102 | mmu-miR-681 |
| 103 | mmu-miR-677-5p |
| 104 | mmu-miR-675-3p |
| 106 | mmu-miR-669e-5p |
| 109 | mmu-miR-5115 |
| 110 | mmu-miR-5105 |
| 111 | mmu-miR-5104 |
| 112 | mmu-miR-503-3p |
| 113 | mmu-miR-489-5p |
| 114 | mmu-miR-485-3p |
| 115 | mmu-miR-483-3p |
| 116 | mmu-miR-467c-3p |
| 117 | mmu-miR-3970 |
| 118 | mmu-miR-3969 |
| 120 | mmu-miR-376c-5p |
| 121 | mmu-miR-375-5p |
| 128 | mmu-miR-30b-5p |
| 130 | mmu-miR-3057-3p |
| 132 | mmu-miR-20a-5p |
| 134 | mmu-miR-1942 |
| 136 | mmu-miR-1903 |
| 137 | mmu-miR-1901 |
| 138 | mmu-miR-1843b-3p |
| 141 | mmu-miR-1264-3p |
| 142 | mmu-miR-1194 |
| 143 | mmu-miR-1188-3p |
| 147 | mmu-miR-30c-1-3p |
| 151 | mmu-miR-92b-3p |
| 152 | mmu-miR-879-3p |
| 155 | mmu-miR-764-5p |
| 158 | mmu-miR-720 |
| 163 | mmu-miR-702 |
| 165 | mmu-miR-669d-5p |
| 171 | mmu-miR-568 |
| 174 | mmu-miR-5621-5p |
| 182 | mmu-miR-5114 |
| 183 | mmu-miR-5106 |
| 185 | mmu-miR-5097 |
| 186 | mmu-miR-5046 |
| 188 | mmu-miR-488-5p |
| 190 | mmu-miR-467d-5p |
| 201 | mmu-miR-351-3p |
| 203 | mmu-miR-344d-1-5p |
| 207 | mmu-miR-330-5p |
| 211 | mmu-miR-3104-3p |
| 212 | mmu-miR-3102-3p.2-3p |
| 214 | mmu-miR-3100-3p |
| 217 | mmu-miR-3093-3p |
| 222 | mmu-miR-3075-3p |
| 223 | mmu-miR-3073b-3p |
| 228 | mmu-miR-3065-5p |
| 230 | mmu-miR-3061-5p |
| 233 | mmu-miR-302a-5p |
| 238 | mmu-miR-29a-3p |
| 239 | mmu-miR-299-3p |
| 240 | mmu-miR-294-5p |
| 254 | mmu-miR-1966 |
| 255 | mmu-miR-1963 |
| 269 | mmu-miR-132-5p |
| 276 | mmu-miR-1231-5p |
| 278 | mmu-miR-1196-5p |
| 279 | mmu-miR-1193-5p |
| 285 | mmu-let-7e-5p |
| 294 | mmu-miR-30c-5p |

The miRNAs of groups 2 (table 2) and 3 (table 3) were found to influence cell survival either by suppressing cell death (group 2) or by promoting apoptosis or necrosis (group 3). Cell death within the culture does not only reduce the number of producing cells but also provides a significant burden to the entire culture. Dead cells remain within the culture as debris, which increases cellular stress and can even become toxic at higher concentrations. Accordingly, cell debris needs to be removed from the cultures, which disturbs the culture conditions and provides physical stress to the cells, all of which finally results in a reduced productivity. Therefore, for suppressing cell death, the nucleic acid construct encodes for a miRNA of group 2, which are suitable to directly inhibit apoptosis. Of these miRNAs, the miRNAs of group 10 consisting of SEQ ID NO.: 3, 7, 20, 54, 59, 73, 94, 145, 159, 175, 176, 178, 179, 199, 206, 248, 251, 252, 266 and 272 were found to show the most prominent effect on cell survival. Accordingly, the second region preferably encodes for at least one miRNA selected from group 10. The miRNAs of group 3 (table 3) were found to promote cell death, in particular apoptosis (group 6; table 6) or necrosis (group 7; table 7). Thus, inhibition of these miRNAs is suitable for suppressing cell death. Within group 3, the miRNAs of group 11 consisting of SEQ ID NO.: 297, 305, 307, 311, 312, 313, 321, 330, 331, 335, 336, 340, 345, 351, 359, 405, 412, 458, 510 and 608 were found to have the most prominent cell death inducing effect, such that an inhibition of these miRNAs is most preferred. Accordingly, the second region preferably encodes for at least one miRNA-inhibitor inhibiting a miRNA selected from group 11.

TABLE 2
miRNAs suppressing apoptosis

| SEQ ID NO.: | miRNA |
|---|---|
| 1 | mmu-miR-99b-3p |
| 2 | mmu-miR-767 |
| 3 | mmu-miR-30a-5p |
| 4 | mmu-miR-3062-5p |
| 5 | mmu-miR-291b-3p |
| 6 | mmu-miR-200a-5p |
| 7 | mmu-miR-1a-3p |
| 8 | mmu-miR-135a-1-3p |
| 9 | mmu-miR-743a-5p |
| 20 | mmu-miR-30d-5p |
| 24 | mmu-miR-878-3p |
| 31 | mmu-miR-669f-3p |
| 33 | mmu-miR-669a-3-3p |
| 50 | mmu-miR-466f-5p |
| 52 | mmu-miR-463-5p |
| 54 | mmu-miR-382-5p |
| 59 | mmu-miR-329-3p |
| 60 | mmu-miR-323-5p |
| 61 | mmu-miR-322-3p |
| 64 | mmu-miR-30e-5p |
| 68 | mmu-miR-3076-3p |
| 71 | mmu-miR-3058-5p |
| 73 | mmu-miR-2861 |
| 74 | mmu-miR-219-1-3p |
| 79 | mmu-miR-205-5p |
| 85 | mmu-miR-1899 |
| 87 | mmu-miR-1896 |
| 89 | mmu-miR-155-3p |
| 92 | mmu-miR-141-5p |
| 94 | mmu-miR-136-3p |
| 95 | mmu-miR-1247-5p |
| 97 | mmu-miR-106a-3p |
| 98 | mmu-let-7a-1-3p |
| 145 | mmu-miR-126-3p |
| 153 | mmu-miR-872-5p |
| 154 | mmu-miR-871-5p |
| 156 | mmu-miR-760-3p |
| 159 | mmu-miR-717 |
| 161 | mmu-miR-711 |
| 166 | mmu-miR-669c-5p |
| 167 | mmu-miR-592-5p |
| 168 | mmu-miR-590-5p |
| 169 | mmu-miR-590-3p |
| 170 | mmu-miR-574-5p |
| 172 | mmu-miR-5626-5p |
| 175 | mmu-miR-551b-5p |
| 176 | mmu-miR-551b-3p |
| 178 | mmu-miR-543-3p |
| 179 | mmu-miR-542-5p |
| 180 | mmu-miR-5136 |
| 181 | mmu-miR-5117-5p |
| 184 | mmu-miR-5100 |
| 191 | mmu-miR-453 |
| 192 | mmu-miR-452-5p |
| 194 | mmu-miR-429-3p |
| 195 | mmu-miR-423-5p |
| 196 | mmu-miR-421-3p |
| 197 | mmu-miR-379-3p |
| 199 | mmu-miR-367-3p |
| 200 | mmu-miR-365-2-5p |
| 204 | mmu-miR-342-5p |
| 205 | mmu-miR-340-3p |
| 206 | mmu-miR-33-3p |
| 208 | mmu-miR-31-5p |
| 209 | mmu-miR-3110-3p |
| 210 | mmu-miR-3109-3p |
| 213 | mmu-miR-3100-5p |
| 216 | mmu-miR-3094-3p |
| 219 | mmu-miR-3088-3p |
| 220 | mmu-miR-3081-5p |
| 221 | mmu-miR-3076-5p |
| 224 | mmu-miR-3072-5p |
| 225 | mmu-miR-3071-3p |
| 226 | mmu-miR-3067-5p |
| 227 | mmu-miR-3066-3p |
| 229 | mmu-miR-3062-3p |
| 231 | mmu-miR-3059-5p |
| 236 | mmu-miR-300-5p |
| 237 | mmu-miR-29b-1-5p |
| 241 | mmu-miR-293-5p |
| 242 | mmu-miR-293-3p |
| 243 | mmu-miR-23a-5p |
| 245 | mmu-miR-223-3p |
| 247 | mmu-miR-217-3p |
| 248 | mmu-miR-211-3p |
| 251 | mmu-miR-200c-5p |
| 252 | mmu-miR-19a-3p |
| 253 | mmu-miR-196a-2-3p |
| 256 | mmu-miR-1952 |
| 257 | mmu-miR-194-1-3p |
| 258 | mmu-miR-193b-5p |
| 259 | mmu-miR-1935 |
| 260 | mmu-miR-1934-5p |
| 262 | mmu-miR-1902 |
| 265 | mmu-miR-188-3p |
| 266 | mmu-miR-182-3p |
| 268 | mmu-miR-134-3p |
| 271 | mmu-miR-128-2-5p |
| 272 | mmu-miR-128-1-5p |
| 273 | mmu-miR-127-5p |
| 274 | mmu-miR-127-3p |
| 277 | mmu-miR-1197-5p |
| 280 | mmu-miR-1191 |
| 282 | mmu-miR-101a-5p |
| 284 | mmu-let-7g-3p |
| 289 | mmu-miR-10b-5p |
| 293 | mmu-miR-221-3p |
| 295 | mmu-miR-346-3p |

TABLE 3
miRNAs promoting cell death

| SEQ ID NO.: | miRNA |
|---|---|
| 296 | mmu-miR-9-5p |
| 297 | mmu-miR-133a-3p |
| 298 | mmu-miR-134-5p |
| 299 | mmu-miR-135a-5p |
| 300 | mmu-miR-137-3p |
| 301 | mmu-miR-154-5p |
| 302 | mmu-miR-183-5p |
| 303 | mmu-miR-185-5p |
| 304 | mmu-let-7d-3p |
| 305 | mmu-miR-29c-3p |
| 306 | mmu-miR-337-3p |
| 307 | mmu-miR-28-5p |
| 308 | mmu-miR-218-5p |
| 309 | mmu-miR-33-5p |
| 310 | mmu-miR-378-5p |
| 311 | mmu-miR-410-3p |
| 312 | mmu-miR-540-3p |
| 313 | mmu-miR-690 |
| 314 | mmu-miR-133a-5p |
| 315 | mmu-miR-673-5p |
| 316 | mmu-miR-744-5p |
| 317 | mmu-miR-183-3p |
| 318 | mmu-miR-29a-5p |
| 319 | mmu-miR-338-5p |
| 320 | mmu-miR-466a-5p |
| 321 | mmu-miR-882 |
| 322 | mmu-miR-466e-5p |
| 323 | mmu-miR-466g |
| 324 | mmu-miR-466j |
| 325 | mmu-miR-467g |
| 326 | mmu-miR-1906 |
| 327 | mmu-miR-1904 |
| 328 | mmu-miR-1943-5p |
| 329 | mmu-miR-1962 |
| 330 | mmu-miR-1839-5p |
| 331 | mmu-miR-3064-5p |
| 332 | mmu-miR-3068-3p |
| 333 | mmu-miR-3073-3p |
| 334 | mmu-miR-3091-5p |
| 335 | mmu-miR-3098-5p |
| 336 | mmu-miR-344c-5p |
| 337 | mmu-miR-3102-3p |
| 338 | mmu-miR-3104-5p |
| 339 | mmu-miR-3112-3p |
| 340 | mmu-miR-192-3p |
| 341 | mmu-miR-103-1-5p |
| 342 | mmu-miR-135a-2-3p |
| 343 | mmu-miR-452-3p |
| 344 | mmu-miR-669f-5p |
| 345 | mmu-miR-1948-5p |
| 346 | mmu-miR-1964-5p |
| 347 | mmu-miR-3096b-3p |
| 348 | mmu-miR-3968 |
| 349 | mmu-miR-5101 |
| 350 | mmu-miR-5709 |
| 351 | mmu-miR-3070a-5p // |
| | mmu-miR-3070b-5p |
| 352 | mmu-miR-669m-5p // |
| | mmu-miR-466m-5p |
| 353 | mmu-miR-706 |
| 354 | mmu-let-7i-5p |
| 355 | mmu-miR-101a-3p |
| 356 | mmu-miR-125a-5p |
| 357 | mmu-miR-152-3p |
| 358 | mmu-miR-201-5p |
| 359 | mmu-miR-202-3p |
| 360 | mmu-miR-290-5p |
| 361 | mmu-miR-34c-5p |
| 362 | mmu-let-7b-5p |
| 363 | mmu-miR-351-5p |
| 364 | mmu-miR-135b-5p |
| 365 | mmu-miR-181c-5p |
| 366 | mmu-miR-217-5p |
| 367 | mmu-miR-380-3p |
| 368 | mmu-miR-215-5p |
| 369 | mmu-miR-448-3p |
| 370 | mmu-miR-449a-5p |
| 371 | mmu-miR-547-3p |
| 372 | mmu-miR-494-3p |
| 373 | mmu-miR-302c-5p |
| 374 | mmu-miR-302c-3p |
| 375 | mmu-miR-679-5p |
| 376 | mmu-miR-683 |
| 377 | mmu-miR-686 |
| 378 | mmu-miR-146b-5p |
| 379 | mmu-miR-467b-3p |
| 380 | mmu-miR-455-5p |
| 381 | mmu-miR-698 |
| 382 | mmu-miR-706 |
| 383 | mmu-miR-707 |
| 384 | mmu-miR-714 |
| 385 | mmu-miR-501-3p |
| 386 | mmu-miR-450b-3p |
| 387 | mmu-miR-505-3p |
| 388 | mmu-miR-718 |
| 389 | mmu-miR-675-5p |
| 390 | mmu-miR-374-3p |
| 391 | mmu-miR-665-3p |
| 392 | mmu-miR-758-3p |
| 393 | mmu-miR-763 |
| 394 | mmu-miR-202-5p |
| 395 | mmu-miR-15a-3p |
| 396 | mmu-miR-20a-3p |
| 397 | mmu-miR-31-3p |
| 398 | mmu-miR-93-3p |
| 399 | mmu-miR-337-5p |
| 400 | mmu-miR-339-3p |
| 401 | mmu-miR-345-3p |
| 402 | mmu-miR-20b-3p |
| 403 | mmu-miR-666-3p |
| 404 | mmu-miR-743b-5p |
| 405 | mmu-miR-883a-3p |
| 406 | mmu-miR-876-3p |
| 407 | mmu-miR-327 |
| 408 | mmu-miR-466b-3p // |
| | mmu-miR-466c-3p // |
| | mmu-miR-466p-3p |
| 409 | mmu-miR-467c-5p |
| 410 | mmu-miR-493-3p |
| 411 | mmu-miR-509-5p |
| 412 | mmu-miR-654-5p |
| 413 | mmu-miR-449b |
| 414 | mmu-miR-669k-3p |
| 415 | mmu-miR-1186 |
| 416 | mmu-miR-1187 |
| 417 | mmu-miR-669h-5p |
| 418 | mmu-miR-1195 |
| 419 | mmu-miR-1198-5p |
| 420 | mmu-miR-1897-5p |
| 421 | mmu-miR-1905 |
| 422 | mmu-miR-1907 |
| 423 | mmu-miR-1894-3p |
| 424 | mmu-miR-1933-5p |
| 425 | mmu-miR-1947-5p |
| 426 | mmu-miR-1948-3p |
| 427 | mmu-miR-1960 |
| 428 | mmu-miR-1946b |
| 429 | mmu-miR-1970 |
| 430 | mmu-miR-1971 |
| 431 | mmu-miR-1982-5p |
| 432 | mmu-miR-2139 |
| 433 | mmu-miR-1249-5p |
| 434 | mmu-miR-3099-3p |
| 435 | mmu-miR-3106-5p |
| 436 | mmu-miR-3106-3p |
| 437 | mmu-miR-3057-5p |
| 438 | mmu-miR-3061-3p |
| 439 | mmu-miR-3063-3p |
| 440 | mmu-miR-3069-5p |
| 441 | mmu-miR-3073-5p |
| 442 | mmu-miR-3079-5p |
| 443 | mmu-miR-3082-3p |
| 444 | mmu-miR-3084-5p |
| 445 | mmu-miR-466m-3p |
| 446 | mmu-miR-466n-5p |
| 447 | mmu-miR-466n-3p |
| 448 | mmu-miR-3092-5p |
| 449 | mmu-miR-3092-3p |
| 450 | mmu-miR-3096-5p |
| 451 | mmu-miR-3097-5p |
| 452 | mmu-miR-3097-3p |
| 453 | mmu-miR-3102-5p |
| 454 | mmu-miR-3102-5p.2-5p |
| 455 | mmu-miR-3108-5p |
| 456 | mmu-miR-3109-5p |
| 457 | mmu-miR-374c-5p |
| 458 | mmu-miR-1912-3p |
| 459 | mmu-miR-3471 |
| 460 | mmu-miR-1186b |
| 461 | mmu-miR-3474 |
| 462 | mmu-miR-137-5p |
| 463 | mmu-miR-146a-3p |
| 464 | mmu-miR-153-5p |
| 465 | mmu-miR-196a-1-3p |
| 466 | mmu-miR-1a-2-5p |
| 467 | mmu-miR-25-5p |
| 468 | mmu-miR-29b-2-5p |
| 469 | mmu-miR-92a-1-5p |
| 470 | mmu-miR-181b-1-3p |
| 471 | mmu-miR-133b-5p |
| 472 | mmu-miR-448-5p |
| 473 | mmu-miR-471-3p |
| 474 | mmu-miR-541-3p |
| 475 | mmu-miR-367-5p |
| 476 | mmu-miR-487b-5p |
| 477 | mmu-miR-669c-3p |
| 478 | mmu-miR-499-3p |
| 479 | mmu-miR-701-3p |
| 480 | mmu-miR-181d-3p |
| 481 | mmu-miR-466h-3p |
| 482 | mmu-miR-493-5p |
| 483 | mmu-miR-653-3p |
| 484 | mmu-miR-669e-3p |
| 485 | mmu-miR-1199-3p |
| 486 | mmu-miR-1947-3p |
| 487 | mmu-miR-1955-3p |
| 488 | mmu-miR-664-5p |
| 489 | mmu-miR-3964 |
| 490 | mmu-miR-3473b |
| 491 | mmu-miR-3473c |
| 492 | mmu-miR-5109 |
| 493 | mmu-miR-5118 |
| 494 | mmu-miR-5120 |
| 495 | mmu-miR-5121 |
| 496 | mmu-miR-3544-3p |
| 497 | mmu-miR-5615-3p |
| 498 | mmu-miR-1231-3p |
| 499 | mmu-miR-5616-3p |
| 500 | mmu-miR-5617-3p |
| 501 | mmu-miR-3073b-5p |
| 502 | mmu-miR-5710 |
| 503 | mmu-miR-1929-3p |
| 504 | mmu-miR-669a-5p // |
| | mmu-miR-669p-5p |
| 505 | mmu-miR-466b-5p // |
| | mmu-miR-466o-5p |
| 506 | mmu-miR-344e-5p // |
| | mmu-miR-344h-5p |
| 507 | mmu-miR-96-5p |
| 508 | mmu-miR-200c-3p |
| 509 | mmu-miR-216a-5p |
| 510 | mmu-miR-761 |
| 511 | mmu-miR-18a-3p |
| 512 | mmu-miR-466k |
| 513 | mmu-miR-467h |
| 514 | mmu-miR-1955-5p |
| 515 | mmu-miR-3096-3p |
| 605 | mmu-let-7f-5p |
| 607 | mmu-miR-24-3p |
| 608 | mmu-miR-298-3p |
| 609 | mmu-miR-7b-5p |

The miRNAs of group 4 (table 4) were found to promote proliferation, whereas those miRNAs of group 5 (table 5) reduced cell division. Thus, expression of miRNAs of group 4 and inhibition of miRNAs of group 5 is suitable for promoting proliferation, whereas expression of miRNAs of group 5 and inhibition of miRNAs of group 4 is suitable for inhibiting proliferation. Besides the cellular production of each single cell, the number of cells present in a culture determines the yield of biomolecule that can be harvested. Therefore, stimulating cell proliferation can be desired for increasing the size of the producing culture, in particular if slowly growing cells are used or when starting a cell culture. On the other hand, once a culture has reached an optimal cell density, inhibiting cell proliferation may be desired. For dividing, a cell needs to roughly duplicate almost all components including membrane, cell nucleus and further organelles. This consumes energy and protein translation capacity, which is then not provided for production of the biomolecule of interest. Therefore, inhibiting cell proliferation can be desired, in particular once an optimal culture size is reached. Of the miRNAs of group 4, miRNAs of group 12 consisting of SEQ ID NO.: 5, 7, 22, 30, 35, 43, 68, 72, 78, 84, 96, 146, 148, 160, 173, 177, 198, 202, 232, 234, 244, 267 and 283, had the most prominent effect on cell proliferation and of those miRNAs found to repress proliferation, the miRNAs of group 13 consisting of SEQ ID NO.: 517, 523, 526, 529, 531, 533, 537, 548, 550, 558, 560, 561, 563, 566, 567, 571, 575, 577, 583, 591, 600, 601 and 604 were most effective. Accordingly, for promoting cell proliferation, the third region preferably encodes for a miRNA selected from group 12 and/or for a miRNA-inhibitor inhibiting a miRNA selected from group 13. For inhibiting cell proliferation, the third region preferably encodes for a miRNA of group 13 and/or for a miRNA-inhibitor inhibiting a miRNA of group 12.

TABLE 4
miRNAs promoting proliferation

| SEQ ID NO.: | miRNA |
|---|---|
| 5 | mmu-miR-291b-3p |
| 7 | mmu-miR-1a-3p |
| 68 | mmu-miR-3076-3p |
| 79 | mmu-miR-205-5p |
| 94 | mmu-miR-136-3p |
| 10 | mmu-miR-694 |
| 11 | mmu-miR-674-3p |
| 12 | mmu-miR-669d-3p |
| 13 | mmu-miR-301b-5p |
| 14 | mmu-miR-212-5p |
| 15 | mmu-miR-203-5p |
| 16 | mmu-miR-200b-5p |
| 17 | mmu-miR-200a-3p |
| 18 | mmu-miR-1968-5p |
| 19 | mmu-miR-150-3p |
| 22 | mmu-miR-880-5p |
| 23 | mmu-miR-878-5p |
| 30 | mmu-miR-684 |
| 35 | mmu-miR-582-5p |
| 36 | mmu-miR-582-3p |
| 38 | mmu-miR-540-5p |
| 43 | mmu-miR-5122 |
| 65 | mmu-miR-3096b-5p |
| 72 | mmu-miR-294-3p |
| 78 | mmu-miR-206-3p |
| 84 | mmu-miR-1930-3p |
| 96 | mmu-miR-1190 |
| 105 | mmu-miR-669I-3p |
| 107 | mmu-miR-539-3p |
| 108 | mmu-miR-5123 |
| 119 | mmu-miR-381-3p |
| 122 | mmu-miR-370-5p |
| 123 | mmu-miR-363-3p |
| 124 | mmu-miR-350-3p |
| 125 | mmu-miR-344h-3p |
| 126 | mmu-miR-330-3p |
| 127 | mmu-miR-3110-5p |
| 129 | mmu-miR-3070a-3p |
| 131 | mmu-miR-224-3p |
| 133 | mmu-miR-1961 |
| 135 | mmu-miR-1931 |
| 139 | mmu-miR-148a-5p |
| 140 | mmu-miR-130b-5p |
| 144 | mmu-miR-125b-5p |
| 146 | mmu-miR-27a-3p |
| 148 | mmu-miR-99a-5p |
| 149 | mmu-miR-99a-3p |
| 150 | mmu-miR-93-5p |
| 160 | mmu-miR-712-5p |
| 162 | mmu-miR-709 |
| 164 | mmu-miR-676-3p |
| 173 | mmu-miR-5622-5p |
| 177 | mmu-miR-544-5p |
| 187 | mmu-miR-491-3p |
| 189 | mmu-miR-488-3p |
| 193 | mmu-miR-431-5p |
| 198 | mmu-miR-376b-5p |
| 202 | mmu-miR-3470a |
| 215 | mmu-miR-3095-5p |
| 218 | mmu-miR-3089-5p |
| 232 | mmu-miR-302b-3p |
| 234 | mmu-miR-302a-3p |
| 235 | mmu-miR-301a-5p |
| 244 | mmu-miR-224-5p |
| 246 | mmu-miR-219-5p |
| 249 | mmu-miR-208b-5p |
| 250 | mmu-miR-208a-3p |
| 261 | mmu-miR-1933-3p |
| 263 | mmu-miR-18a-5p |
| 264 | mmu-miR-1894-5p |
| 267 | mmu-miR-148a-3p |
| 270 | mmu-miR-132-3p |
| 275 | mmu-miR-1251-3p |
| 281 | mmu-miR-103-2-5p |
| 283 | mmu-miR-100-3p |
| 287 | mmu-miR-107-5p |
| 288 | mmu-miR-10a-3p |
| 291 | mmu-miR-191-5p |

TABLE 5
miRNAs reducing cell division

| SEQ ID NO.: | miRNA |
|---|---|
| 516 | mmu-miR-9-3p |
| 517 | mmu-miR-136-5p |
| 518 | mmu-miR-155-5p |
| 519 | mmu-miR-193-3p |
| 520 | mmu-miR-204-5p |
| 521 | mmu-miR-143-3p |
| 522 | mmu-let-7c-5p |
| 523 | mmu-let-7e-5p |
| 524 | mmu-miR-29a-3p |
| 525 | mmu-miR-34a-5p |
| 526 | mmu-miR-320-3p |
| 527 | mmu-miR-379-5p |
| 528 | mmu-miR-196b-5p |
| 529 | mmu-miR-484 |
| 530 | mmu-miR-546 |
| 531 | mmu-miR-488-5p |
| 532 | mmu-miR-696 |
| 533 | mmu-miR-720 |
| 534 | mmu-miR-697 |
| 535 | mmu-miR-713 |
| 536 | mmu-miR-501-5p |
| 537 | mmu-miR-666-5p |
| 538 | mmu-miR-764-5p |
| 539 | mmu-miR-804 |
| 540 | mmu-miR-145-3p |
| 541 | mmu-miR-294-5p |
| 542 | mmu-miR-299-3p |
| 543 | mmu-miR-302a-5p |
| 544 | mmu-miR-330-5p |
| 545 | mmu-miR-340-5p |
| 546 | mmu-miR-139-3p |
| 547 | mmu-miR-362-3p |
| 548 | mmu-miR-409-5p |
| 549 | mmu-miR-671-3p |
| 550 | mmu-miR-881-3p |
| 551 | mmu-miR-297c-5p |
| 552 | mmu-miR-466h-5p |
| 553 | mmu-miR-467d-5p |
| 554 | mmu-miR-568 |
| 555 | mmu-miR-872-3p |
| 556 | mmu-miR-669d-5p |
| 557 | mmu-miR-669e-5p |
| 558 | mmu-miR-1197-3p |
| 559 | mmu-miR-1941-5p |
| 560 | mmu-miR-1953 |
| 561 | mmu-miR-1963 |
| 562 | mmu-miR-1966 |
| 563 | mmu-miR-1249-3p |
| 564 | mmu-miR-3058-3p |
| 565 | mmu-miR-344d-1-5p |
| 566 | mmu-miR-3060-3p |
| 567 | mmu-miR-3061-5p |
| 568 | mmu-miR-3065-5p |
| 569 | mmu-miR-3074-5p |
| 570 | mmu-miR-669d-2-3p |
| 571 | mmu-miR-3093-3p |
| 572 | mmu-miR-3100-3p |
| 573 | mmu-miR-344g-5p |
| 574 | mmu-miR-3102-3p.2-3p |
| 575 | mmu-miR-3104-3p |
| 576 | mmu-miR-3107-3p |
| 577 | mmu-miR-3112-5p |
| 578 | mmu-miR-130a-5p |
| 579 | mmu-miR-132-5p |
| 580 | mmu-miR-187-5p |
| 581 | mmu-let-7a-2-3p |
| 582 | mmu-miR-351-3p |
| 583 | mmu-miR-215-3p |
| 584 | mmu-miR-412-5p |
| 585 | mmu-miR-592-3p |
| 586 | mmu-miR-760-5p |
| 587 | mmu-miR-497-3p |
| 588 | mmu-miR-700-5p |
| 589 | mmu-miR-871-3p |
| 590 | mmu-miR-874-5p |
| 591 | mmu-miR-504-3p |
| 592 | mmu-miR-669k-5p |
| 593 | mmu-miR-466i-5p |
| 594 | mmu-miR-1193-5p |
| 595 | mmu-miR-5098 |
| 596 | mmu-miR-5106 |
| 597 | mmu-miR-5114 |
| 598 | mmu-miR-5134 |
| 599 | mmu-miR-1231-5p |
| 600 | mmu-miR-5617-5p |
| 601 | mmu-miR-5621-5p |
| 602 | mmu-miR-5621-3p |
| 603 | mmu-miR-5623-5p |
| 604 | mmu-miR-3073b-3p |
| 606 | mmu-miR-24-2-5p |

TABLE 6
miRNAs promoting apoptosis

| SEQ ID NO.: | miRNA |
|---|---|
| 296 | mmu-miR-9-5p |
| 297 | mmu-miR-133a-3p |
| 298 | mmu-miR-134-5p |
| 299 | mmu-miR-135a-5p |
| 300 | mmu-miR-137-3p |
| 301 | mmu-miR-154-5p |
| 302 | mmu-miR-183-5p |
| 303 | mmu-miR-185-5p |
| 304 | mmu-let-7d-3p |
| 305 | mmu-miR-29c-3p |
| 306 | mmu-miR-337-3p |
| 307 | mmu-miR-28-5p |
| 308 | mmu-miR-218-5p |
| 309 | mmu-miR-33-5p |
| 310 | mmu-miR-378-5p |
| 311 | mmu-miR-410-3p |
| 312 | mmu-miR-540-3p |
| 313 | mmu-miR-690 |
| 314 | mmu-miR-133a-5p |
| 315 | mmu-miR-673-5p |
| 316 | mmu-miR-744-5p |
| 317 | mmu-miR-183-3p |
| 318 | mmu-miR-29a-5p |
| 319 | mmu-miR-338-5p |
| 320 | mmu-miR-466a-5p |
| 321 | mmu-miR-882 |
| 322 | mmu-miR-466e-5p |
| 323 | mmu-miR-466g |
| 324 | mmu-miR-466j |
| 325 | mmu-miR-467g |
| 326 | mmu-miR-1906 |
| 327 | mmu-miR-1904 |
| 328 | mmu-miR-1943-5p |
| 329 | mmu-miR-1962 |
| 330 | mmu-miR-1839-5p |
| 331 | mmu-miR-3064-5p |
| 332 | mmu-miR-3068-3p |
| 333 | mmu-miR-3073-3p |
| 334 | mmu-miR-3091-5p |
| 335 | mmu-miR-3098-5p |
| 336 | mmu-miR-344c-5p |
| 337 | mmu-miR-3102-3p |
| 338 | mmu-miR-3104-5p |
| 339 | mmu-miR-3112-3p |
| 340 | mmu-miR-192-3p |
| 341 | mmu-miR-103-1-5p |
| 342 | mmu-miR-135a-2-3p |
| 343 | mmu-miR-452-3p |
| 344 | mmu-miR-669f-5p |
| 345 | mmu-miR-1948-5p |
| 346 | mmu-miR-1964-5p |
| 347 | mmu-miR-3096b-3p |
| 348 | mmu-miR-3968 |
| 349 | mmu-miR-5101 |
| 350 | mmu-miR-5709 |
| 351 | mmu-miR-3070a-5p // |
| | mmu-miR-3070b-5p |
| 352 | mmu-miR-669m-5p // |
| | mmu-miR-466m-5p |
| 507 | mmu-miR-96-5p |
| 508 | mmu-miR-200c-3p |
| 509 | mmu-miR-216a-5p |
| 510 | mmu-miR-761 |
| 511 | mmu-miR-18a-3p |
| 512 | mmu-miR-466k |
| 513 | mmu-miR-467h |
| 514 | mmu-miR-1955-5p |
| 515 | mmu-miR-3096-3p |
| 605 | mmu-let-7f-5p |
| 608 | mmu-miR-298-3p |

TABLE 7
miRNAs promoting necrosis

| SEQ ID NO.: | miRNA |
|---|---|
| 296 | mmu-miR-9-5p |
| 297 | mmu-miR-133a-3p |
| 298 | mmu-miR-134-5p |
| 299 | mmu-miR-135a-5p |
| 300 | mmu-miR-137-3p |
| 301 | mmu-miR-154-5p |
| 302 | mmu-miR-183-5p |
| 303 | mmu-miR-185-5p |
| 304 | mmu-let-7d-3p |
| 305 | mmu-miR-29c-3p |
| 306 | mmu-miR-337-3p |
| 307 | mmu-miR-28-5p |
| 308 | mmu-miR-218-5p |
| 309 | mmu-miR-33-5p |
| 310 | mmu-miR-378-5p |
| 311 | mmu-miR-410-3p |
| 312 | mmu-miR-540-3p |
| 313 | mmu-miR-690 |
| 314 | mmu-miR-133a-5p |
| 315 | mmu-miR-673-5p |
| 316 | mmu-miR-744-5p |
| 317 | mmu-miR-183-3p |
| 318 | mmu-miR-29a-5p |
| 319 | mmu-miR-338-5p |
| 320 | mmu-miR-466a-5p |
| 321 | mmu-miR-882 |
| 322 | mmu-miR-466e-5p |
| 323 | mmu-miR-466g |
| 324 | mmu-miR-466j |
| 325 | mmu-miR-467g |
| 326 | mmu-miR-1906 |
| 327 | mmu-miR-1904 |
| 328 | mmu-miR-1943-5p |
| 329 | mmu-miR-1962 |
| 330 | mmu-miR-1839-5p |
| 331 | mmu-miR-3064-5p |
| 332 | mmu-miR-3068-3p |
| 333 | mmu-miR-3073-3p |
| 334 | mmu-miR-3091-5p |
| 335 | mmu-miR-3098-5p |
| 336 | mmu-miR-344c-5p |
| 337 | mmu-miR-3102-3p |
| 338 | mmu-miR-3104-5p |
| 339 | mmu-miR-3112-3p |
| 340 | mmu-miR-192-3p |
| 341 | mmu-miR-103-1-5p |
| 342 | mmu-miR-135a-2-3p |
| 343 | mmu-miR-452-3p |
| 344 | mmu-miR-669f-5p |
| 345 | mmu-miR-1948-5p |
| 346 | mmu-miR-1964-5p |
| 347 | mmu-miR-3096b-3p |
| 348 | mmu-miR-3968 |
| 349 | mmu-miR-5101 |
| 350 | mmu-miR-5709 |
| 351 | mmu-miR-3070a-5p // |
| | mmu-miR-3070b-5p |
| 352 | mmu-miR-669m-5p // |
| | mmu-miR-466m-5p |
| 353 | mmu-miR-706 |
| 354 | mmu-let-7i-5p |
| 355 | mmu-miR-101a-3p |
| 356 | mmu-miR-125a-5p |
| 357 | mmu-miR-152-3p |
| 358 | mmu-miR-201-5p |
| 359 | mmu-miR-202-3p |
| 360 | mmu-miR-290-5p |
| 361 | mmu-miR-34c-5p |
| 362 | mmu-let-7b-5p |
| 363 | mmu-miR-351-5p |
| 364 | mmu-miR-135b-5p |
| 365 | mmu-miR-181c-5p |
| 366 | mmu-miR-217-5p |
| 367 | mmu-miR-380-3p |
| 368 | mmu-miR-215-5p |
| 369 | mmu-miR-448-3p |
| 370 | mmu-miR-449a-5p |
| 371 | mmu-miR-547-3p |
| 372 | mmu-miR-494-3p |
| 373 | mmu-miR-302c-5p |
| 374 | mmu-miR-302c-3p |
| 375 | mmu-miR-679-5p |
| 376 | mmu-miR-683 |
| 377 | mmu-miR-686 |
| 378 | mmu-miR-146b-5p |
| 379 | mmu-miR-467b-3p |
| 380 | mmu-miR-455-5p |
| 381 | mmu-miR-698 |
| 382 | mmu-miR-706 |
| 383 | mmu-miR-707 |
| 384 | mmu-miR-714 |
| 385 | mmu-miR-501-3p |
| 386 | mmu-miR-450b-3p |
| 387 | mmu-miR-505-3p |
| 388 | mmu-miR-718 |
| 389 | mmu-miR-675-5p |
| 390 | mmu-miR-374-3p |
| 391 | mmu-miR-665-3p |
| 392 | mmu-miR-758-3p |
| 393 | mmu-miR-763 |
| 394 | mmu-miR-202-5p |
| 395 | mmu-miR-15a-3p |
| 396 | mmu-miR-20a-3p |
| 397 | mmu-miR-31-3p |
| 398 | mmu-miR-93-3p |
| 399 | mmu-miR-337-5p |
| 400 | mmu-miR-339-3p |
| 401 | mmu-miR-345-3p |
| 402 | mmu-miR-20b-3p |
| 403 | mmu-miR-666-3p |
| 404 | mmu-miR-743b-5p |
| 405 | mmu-miR-883a-3p |
| 406 | mmu-miR-876-3p |
| 407 | mmu-miR-327 |
| 408 | mmu-miR-466b-3p // |
| | mmu-miR-466c-3p // |
| | mmu-miR-466p-3p |
| 409 | mmu-miR-467c-5p |
| 410 | mmu-miR-493-3p |
| 411 | mmu-miR-509-5p |
| 412 | mmu-miR-654-5p |
| 413 | mmu-miR-449b |
| 414 | mmu-miR-669k-3p |
| 415 | mmu-miR-1186 |
| 416 | mmu-miR-1187 |
| 417 | mmu-miR-669h-5p |
| 418 | mmu-miR-1195 |
| 419 | mmu-miR-1198-5p |
| 420 | mmu-miR-1897-5p |
| 421 | mmu-miR-1905 |
| 422 | mmu-miR-1907 |
| 423 | mmu-miR-1894-3p |
| 424 | mmu-miR-1933-5p |
| 425 | mmu-miR-1947-5p |
| 426 | mmu-miR-1948-3p |
| 427 | mmu-miR-1960 |
| 428 | mmu-miR-1946b |
| 429 | mmu-miR-1970 |
| 430 | mmu-miR-1971 |
| 431 | mmu-miR-1982-5p |
| 432 | mmu-miR-2139 |
| 433 | mmu-miR-1249-5p |
| 434 | mmu-miR-3099-3p |
| 435 | mmu-miR-3106-5p |
| 436 | mmu-miR-3106-3p |
| 437 | mmu-miR-3057-5p |
| 438 | mmu-miR-3061-3p |
| 439 | mmu-miR-3063-3p |
| 440 | mmu-miR-3069-5p |
| 441 | mmu-miR-3073-5p |
| 442 | mmu-miR-3079-5p |
| 443 | mmu-miR-3082-3p |
| 444 | mmu-miR-3084-5p |
| 445 | mmu-miR-466m-3p |
| 446 | mmu-miR-466n-5p |
| 447 | mmu-miR-466n-3p |
| 448 | mmu-miR-3092-5p |
| 449 | mmu-miR-3092-3p |
| 450 | mmu-miR-3096-5p |
| 451 | mmu-miR-3097-5p |
| 452 | mmu-miR-3097-3p |
| 453 | mmu-miR-3102-5p |
| 454 | mmu-miR-3102-5p.2-5p |
| 455 | mmu-miR-3108-5p |
| 456 | mmu-miR-3109-5p |
| 457 | mmu-miR-374c-5p |
| 458 | mmu-miR-1912-3p |
| 459 | mmu-miR-3471 |
| 460 | mmu-miR-1186b |
| 461 | mmu-miR-3474 |
| 462 | mmu-miR-137-5p |
| 463 | mmu-miR-146a-3p |
| 464 | mmu-miR-153-5p |
| 465 | mmu-miR-196a-1-3p |
| 466 | mmu-miR-1a-2-5p |
| 467 | mmu-miR-25-5p |
| 468 | mmu-miR-29b-2-5p |
| 469 | mmu-miR-92a-1-5p |
| 470 | mmu-miR-181b-1-3p |
| 471 | mmu-miR-133b-5p |
| 472 | mmu-miR-448-5p |
| 473 | mmu-miR-471-3p |
| 474 | mmu-miR-541-3p |
| 475 | mmu-miR-367-5p |
| 476 | mmu-miR-487b-5p |
| 477 | mmu-miR-669c-3p |
| 478 | mmu-miR-499-3p |
| 479 | mmu-miR-701-3p |
| 480 | mmu-miR-181d-3p |
| 481 | mmu-miR-466h-3p |
| 482 | mmu-miR-493-5p |
| 483 | mmu-miR-653-3p |
| 484 | mmu-miR-669e-3p |
| 485 | mmu-miR-1199-3p |
| 486 | mmu-miR-1947-3p |
| 487 | mmu-miR-1955-3p |
| 488 | mmu-miR-664-5p |
| 489 | mmu-miR-3964 |
| 490 | mmu-miR-3473b |
| 491 | mmu-miR-3473c |
| 492 | mmu-miR-5109 |
| 493 | mmu-miR-5118 |
| 494 | mmu-miR-5120 |
| 495 | mmu-miR-5121 |
| 496 | mmu-miR-3544-3p |
| 497 | mmu-miR-5615-3p |
| 498 | mmu-miR-1231-3p |
| 499 | mmu-miR-5616-3p |
| 500 | mmu-miR-5617-3p |
| 501 | mmu-miR-3073b-5p |
| 502 | mmu-miR-5710 |
| 503 | mmu-miR-1929-3p |
| 504 | mmu-miR-669a-5p // |
| | mmu-miR-669p-5p |
| 505 | mmu-miR-466b-5p // |
| | mmu-miR-466o-5p |
| 506 | mmu-miR-344e-5p // |
| | mmu-miR-344h-5p |
| 607 | mmu-miR-24-3p |
| 609 | mmu-miR-7b-5p |

The production efficiency and the total output of biomolecules that can be harvested from a cell culture depends on several cellular processes, of which the most important are protein cellular production of the biomolecule (translation/secretion), cell survival and cell proliferation, regulation of which is even interrelated. By introducing at least two miRNAs and/or miRNA-inhibitors that influence different cellular processes, a multitude of cellular pathways can be optimized resulting in an increased yield of the biomolecule of interest. Each of the miRNA and miRNA-inhibitors influences a variety of target genes of several interrelated cellular pathways, thereby influencing the composition of proteins within the cell. In contrast to the overexpression of one or several enzymes for enhancing protein synthesis, shifting the balance within the cells' endogenous protein pool does not withdraw energy from the production of the desired biomolecule. Expressing a miRNA actually reduces translation, thus, releasing energy and protein translation capacity for production of the biomolecule. Moreover, the limiting factor of productivity may differ depending on the biomolecule produced, or may even change during cultivation depending on the culture conditions. By influencing the composition of a large variety of proteins by controlling miRNAs, it is possible to regulate entire pathways. Thereby, it is possible to overcome various limitations, which could not be addressed by overexpressing a single synthesis enzyme or inhibiting or knocking out a single protein degrading enzyme.

The term “biomolecule” as used herein refers to any compound suitable to be produced by a cell and harvested therefrom. Preferably, the biomolecule is a biopharmaceutical, i.e. a pharmaceutical including therapeutics, prophylactics and diagnostics, which is inherently biological in nature and manufactured using biotechnology. Biopharmaceuticals include inter alia antibodies, enzymes, hormones, vaccines but also viruses, e.g. oncolytic viruses and viruses used for gene therapy. Thus, the biopharmaceutical is preferably a recombinant molecule, more preferred a recombinant protein or a recombinant virus.

The term “miRNA-inhibitor” as used herein refers to any compound suitable to specifically reduce the amount of a given miRNA within a cell. miRNA-inhibitors include for example nucleic acid molecules that specifically bind to the miRNA of interest thereby preventing its binding to the target mRNA. Such inhibitors include antagomirs, miRNA sponge and miRNA decoy. Antagomirs are small oligonucleotides that are perfectly complementary to the targeted miRNA, whereas miRNA sponge and RNA decoy are nucleic acid molecules comprising multiple tandem binding sites to the miRNA of interest. Due to the multiple binding sites, the molecules act as strong competitive inhibitors of the miRNA (Ebert and Sharp, 2010). Accordingly, the miRNA-inhibitor is preferably selected from the group consisting of antagomir, miRNA sponge and miRNA decoy. Alternatively, the miRNAs inhibitors may target a regulatory element of the miRNA of interest, e.g. its promoter or enhancer.

In a preferred embodiment, the nucleic acid construct comprises three different regions. By comprising at least one miRNA and/or miRNA-inhibitor influencing each of cellular production, cell death and cell proliferation, the cell's efficiency in producing the biomolecule can be optimized.

In a preferred embodiment, at least one region, preferably each region, encodes for at least two, three, four or five different miRNAs and/or miRNA-inhibitors. Any region may encode for more than one miRNA or miRNA-inhibitor. For example, several miRNAs belonging to the same family and thus targeting related mRNAs, may be comprised. Thereby, it is possible to strengthen the regulation of one particular pathway as e.g. observed by a combined introduction of several members of the miR-30 family.

Alternatively, a variety of miRNAs targeting different pathways may be used to produce a more wide spread effect. Cell processes as proliferation, protein synthesis and cell death are usually regulated by more than one signalling pathway, of which many are interrelated. Thus, targeting several pathways is particularly advantageous in cases in which it is not known which cellular pathways present the limiting factor for biomolecule production. Due to the rather small size of miRNAs, the nucleic acid constructs of the invention may encode for 20 miRNAs and/or miRNA-inhibitors, or even more. The term “region” as used herein refers to sections along the nucleic acid construct comprising a part that is transcribed into a miRNA or a miRNA-inhibitor as e.g. an antagomir or a miRNA decoy. The region may further comprise regulatory elements to control the transcription of the miRNA or miRNA-inhibitor, such as promoters, operators (e.g. enhancers, repressors and insulators), 3′UTR regulatory elements (e.g. siRNA binding sites, miRNA binding sites) or splicing signals.

In a preferred embodiment, the at least two different regions are controlled by different promoters. This is to say that each region comprises its own regulatory elements, such that the transcription of the miRNA and/or miRNA-inhibitor comprised in said region can be regulated independently of the miRNAs and/or miRNA-inhibitors contained in other regions of the construct. This is advantageous if cell proliferation and cellular production should be regulated at different time points during cell culture. When inducing the culture, cell proliferation can be promoted whereas once an optimal cell density is reached, cellular productivity may be enhanced. Regulation of cell death could be specifically induced depending on the state of the culture. Moreover, using independent regulatory elements allows providing the miRNAs and miRNA-inhibitors for different cellular processes at various amounts. For example, miRNAs stimulating cellular production may be set under a strong promoter, whereas those miRNAs or miRNA-inhibitors influencing cell death may be regulated by a weaker promoter.

In a preferred embodiment, the at least two different regions are controlled by one common promoter. This allows a fast and easy preparation of the nucleic acid construct and is preferably applied in cases in which a rather simple regulation already results in satisfying yields of the biomolecule.

In a preferred embodiment, at least one promoter is inducible or inhibitable. Inducible/Inhibitable promoters are characterized in that their activity depends on external circumstances, such as temperature, light, oxygen or the presence of chemical compounds. Using inducible or inhibitable promoters, it is possible to exactly determine the time point during the cell culture when transcription of one or all regions of the construct is initiated and/or terminated. Inducible regulatory elements include for example the tetracycline/doxycycline “Tet-On”-system, inhibitable regulatory elements include for example the “Tet-Off”-system or regulated optogenetic gene expression systems, temperature controlled promotors and TrsR-based systems (quorum sensing based).

In a preferred embodiment, the nucleic acid construct is an expression vector, an episomal vector or a viral vector. For expressing a miRNA and/or a miRNA-inhibitor within a cell, the nucleic acid construct needs to be introduced into the cell. This is possible by different means, for example by transfection, i.e. non-viral methods for transferring a nucleic acid molecule into eukaryotic cells. For such applications, the nucleic acid construct is preferably provided as an expression vector or an episomal vector. Alternatively, the nucleic acid construct can be introduced into a cell by transduction, i.e. by a virus-mediated transfer of the nucleic acid into the cell. For such applications, the nucleic acid construct is preferably provided as a viral vector.

In a further aspect, the invention relates to a cell comprising a nucleic acid construct of the invention. Such cells are suitable for producing a biomolecule, wherein the efficiency of production and the overall yield is optimized by regulating at least two miRNAs involved in different cellular processes. Such cells are preferably used in biopharmaceutical manufacturing.

In a preferred embodiment, the construct is integrated into the cell's genome. By introducing a construct according to the invention into the cell's endogenous genome, a stable cell line for biopharmaceutical manufacturing can be provided. Such cell lines produce biomolecules at constant and reliable amounts and are, thus, particularly preferred for large scale productions, which are usually operated to provide established and highly demanded biopharmaceuticals. Moreover, by integrating the nucleic acid construct into the genome, the probability of loosing the construct during continuous cell proliferation is reduced and the nucleic acid construct is present throughout the entire culture's lifetime. Accordingly, the cell is preferably a stable cell line cell.

In a preferred embodiment, the construct is introduced into the cell by transfection. Transient transfection is an easy and fast way to provide a given cell with new properties. It is neither labour nor cost intensive and does not need extensive selection processes. Introducing the nucleic acid construct by transfection is particularly preferred where biomolecules need to be produced on short term notice or only small amounts of the biomolecule are needed, such that the labour-intensive establishment of a stable cell line would be inefficient.

In a preferred embodiment, a region of the cell's genome encoding for at least one miRNA selected from group 1, 2, 4 and/or 5 is amplified, and/or a region of the cell's genome encoding for at least one miRNA selected from group 3, 4 and/or 5 is deleted or silenced. Besides introducing a nucleic acid construct of the invention into a cell, a miRNA may be provided or inhibited by altering the cells endogenous expression of the miRNA. For increasing the level of a specific miRNA, the region of the cells genome encoding for this miRNA may be amplified. Likewise, the region encoding for an endogenous miRNA may be deleted such that the miRNA is no longer present in the cell. Instead of deleting the gene encoding for the miRNA, the levels of miRNA within the cell may be reduced by silencing, e.g. using competitive inhibitors.

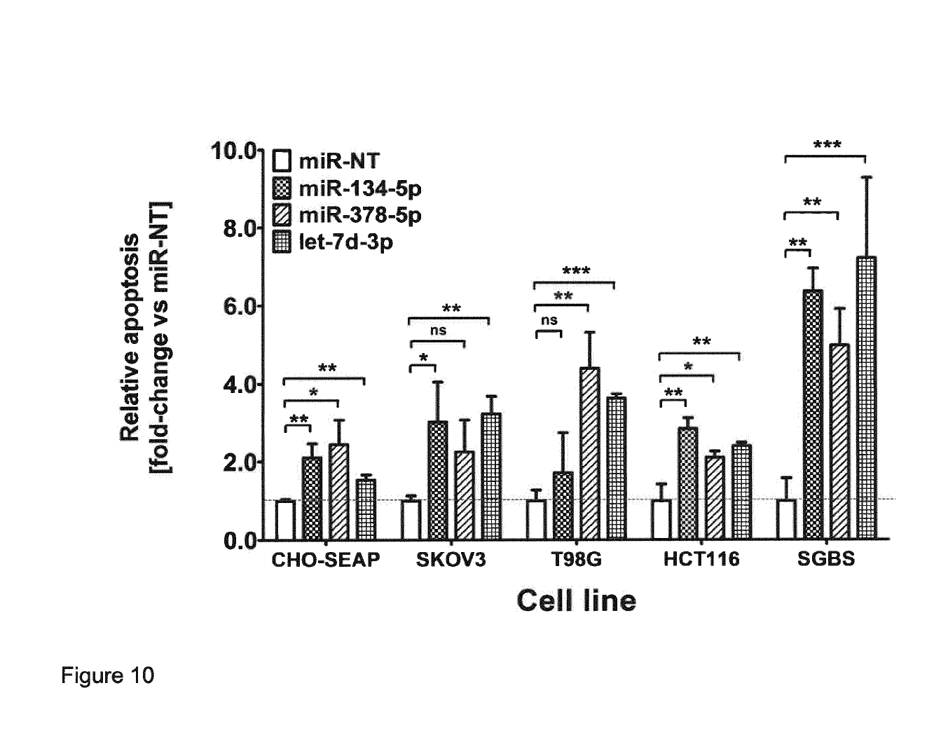

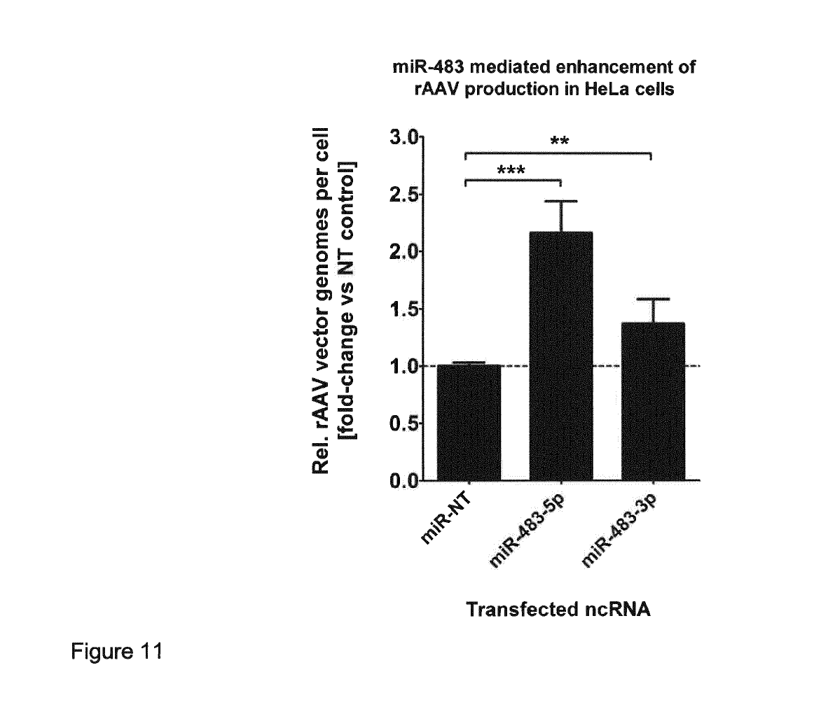

In a preferred embodiment, the cell is a mammalian cell. Mammalian cells are particularly preferred for producing biomolecules of complex structures, as for example proteins comprising sophisticated post-translational modifications. Mammalian cells endogenously comprise the synthesis pathways necessary for generating, folding and modifying complex proteins. A variety of cellular systems derived from different origins as e.g. from hamster, mouse, duck or human are available. Due to the pronounced sequence homology of many genes between different mammalian species, the miRNA, although identified using Chinese hamster ovary (CHO) cells, are suitable to influence cellular parameters determining protein expression, folding, secretion and product quality in cells of other species, in particular other mammalian cells. For example, miRNAs found to have apoptosis promoting effects in CHO cells, were also suitable to induce apoptosis in human tumor and preadipocyte cell lines (FIG. 10).

In a preferred embodiment, the mammalian cell is a Chinese hamster ovary cell (CHO), preferably a CHO-K1 cell, a CHO DG44 cell, a CHO DUKX B11 cell, a CHO dhfr cell or a CHO-S cell.

In a preferred embodiment, the cell is a human cell, preferably a kidney cell, a liver cell, an embryonic retina cell, an amniocytic cell or a mesenchymal stem cell. Cellular production systems derived from human cells are preferred for the production of biomolecules of human origin, in particular if these are intended to be used in human medicine. Marginal alternations of the biomolecules due to incorrect folding of modification may cause the protein to be less active or even to show adverse effects. Moreover, miRNAs identified using CHO cells were found to have similar effects in human cells.

In a preferred embodiment, the cell is an insect cell, preferably a Sf9, Sf21, TriEX™ or a Hi5 cell. Such cell systems are particularly preferred for the production of molecules, e.g. proteins, which originate from other systems, in which they exert essential functions. Expressing such proteins in their natural cellular environment would disturb the cellular processes of the producing cell or even result in cell death. This would significantly impair the production efficiency of the biomolecule, strongly limiting the yield that can be achieved. For example, certain human receptor molecules with significant influence on cellular pathways can be produced in high yields from insect cells, as they do not exert any biological effect in these cells.

In a further aspect, the invention relates to a method for increasing the yield of a biomolecule produced by a cell cultured in vitro comprising at least two steps selected of stimulating cellular production of the biomolecule by increasing the level of at least one miRNA selected from group 1 in the cell, reducing cell death by increasing the level of at least one miRNA selected from group 2 in the cell and/or decreasing the level of at least one miRNA selected from group 3, and regulating proliferation of the cell by increasing the level of at least one miRNA selected from group 4 or 5 in the cell and/or decreasing the level of at least one miRNA selected from group 4 or 5. The term “yield of a biomolecule” as used herein refers to the volumetric productivity of an entire culture, i.e. the total amount of a biomolecule of interest that can be harvested from a culture. For producing the biomolecule, a cell culture is established, preferably from a stable cell line that is adapted to produce the biomolecule of interest. In case the biomolecule is a protein, this may be achieved by introducing one or multiple copies of a gene encoding for the desired protein. By using the described method, at least two cellular processes of cellular production, proliferation and cell survival are influenced via regulating the level of specific miRNAs. By balancing cellular production capacity, cell proliferation and cell survival, it is possible to increase the output of biomolecule without loading the cell with additional translational burden that would increase the cell cultures consumption of energy and nutrients.

In a preferred embodiment, the level of at least one miRNA is increased by overexpressing the miRNA in the cell, by electroporating the cell in the presence of the miRNA or by adding the miRNA and a transfectant to a medium, in which the cell is cultured. Alternatively, the miRNA and the transfectant may be added to a buffer into which the cells are transferred for transfection. For increasing the level of a miRNA within a cell two distinct approaches are available. A nucleic acid molecule encoding for the miRNA may be introduced into a cell such that the cellular transcription machinery expresses the miRNA from the construct. This may be achieved by use of an expression vector or a viral vector that is maintained in the cell as an individual episomal molecule or integrates into the cell's genome, or by use of a stable cell line. Alternatively, the level of a miRNA within a cell may be increased by providing the miRNA as a RNA molecule, e.g. as a pri- or pre-miRNA, a mature miRNA or a miRNA mimic. For example, the RNA molecules are added to the culture together with a transfectant, i.e. lipofectamine (Invitrogen), which contains lipid subunits that form liposomes encapsulating the nucleic acid or miRNA. The liposomes then fuse with the membrane of the cell, such that the nucleic acid becomes introduced into the cytoplasm.

In a preferred embodiment, the level of at least one miRNA is decreased by deleting the region of the cell's genome encoding for the miRNA or regulating its transcription by expressing a miRNA-inhibitor in a cell directed against the miRNA, by electroporating the cell in the presence of the miRNA-inhibitor or by adding a miRNA-inhibitor and a transfectant to a medium, in which the cell is cultured. Alternatively, the miRNA-inhibitor and the transfectant may be added to a buffer into which the cells are transferred for transfection. Reduction of the level of a miRNA may be achieved by various approaches. For example, the endogenous gene encoding for the miRNA may be deleted from the cell's genome. This is preferred when the biomolecule should be produced by a stable cell line. However, this approach may be irreversible in some cases. Alternatively, the endogenous gene encoding for the miRNA may be put under an inducible regulatory element, such that transcription of the miRNA may be determinably activated or inactivated. Besides that, an endogenous miRNA may be also inhibited by providing a competitive inhibitor, e.g. an antagomir or RNA sponge. These may be expressed within the cell upon transfection or may be added as a RNA molecule to the culture together with a transfectant. Instead of a single approach, a combination of different approaches may also be applied.

In a preferred embodiment, cell death is reduced by reducing apoptosis by increasing the level of at least one miRNA selected from group 2 in the cell and/or decreasing the level of at least one miRNA selected from group 6. Two major types of cell death are known, which differ distinctly from each other. Apoptosis, also called programmed cell death, involves a distinct sequence of cellular transformations which is usually initiated as a result from failing cellular processes. Necrosis, in contrast, describes a rather traumatic dissolving of the cell usually initiated by external impacts, e.g. cellular damage. According to cell type and culture conditions one type of cell death may be more prominent than the other. Interestingly, the inventors found several miRNAs involved in regulating both apoptosis and necrosis. Moreover, with respect to apoptosis, inhibiting as well as promoting miRNAs were identified (groups 2 and 6, respectively). In contrast, regarding necrosis, exclusively promoting miRNAs were found (group 7). Depending on the specific cell culture and on the culture conditions, apoptosis or necrosis may be more prevalent during biomolecule production. Accordingly, in a preferred embodiment, cell death is reduced by attenuating necrosis by decreasing the level of at least one miRNA selected from group 7.

In a further aspect, the invention relates to a method for producing a biomolecule in a cell comprising the steps of propagating the cell in a cell culture, increasing the level of at least one miRNA selected from the group consisting of SEQ ID NO.: 1, 2, 3, 4, 5, 6, 7, 8, 9, 10, 11, 12, 13, 14, 15, 16, 17, 18, 19, and 20, and isolating the biomolecule from the cell culture. When revealing miRNAs specifically regulating distinct cellular processes such as cellular production, proliferation and cell survival, the inventors further found certain miRNAs, which influence more than one of these processes. miR-99b-3p (SEQ ID NO.: 1) not only increases the cellular production of a biomolecule, but also shows an anti-apoptotic effect. Similar combined effects were observed for miR-767 (SEQ ID NO.: 2), miR-30a-5p (SEQ ID NO.: 3), miR-3062-5p (SEQ ID NO.: 4), miR-200a-5p (SEQ ID NO.: 6), miR-135a-1-3p (SEQ ID NO.: 8), miR-743a-5p (SEQ ID NO.: 9) and miR-30d-5p (SEQ ID NO.: 20). miR-291b-3p (SEQ ID NO.: 5) and miR-la-3p (SEQ ID NO.: 7) were found to promote both cell survival and cell proliferation resulting in an overall increased yield of the produced biomolecule. Additionally, miR-694 (SEQ ID NO.: 10), miR-674-3p (SEQ ID NO.: 11), miR-669d-3p (SEQ ID NO.: 12); miR-301b-5p (SEQ ID NO.: 13), miR-212-5p (SEQ ID NO.: 14), miR-203-5p (SEQ ID NO.: 15), miR-200b-5p (SEQ ID NO.: 16), miR-200a-3p (SEQ ID NO.: 17), miR-1968-5p (SEQ ID NO.: 18) and miR-150-3p (SEQ ID NO.: 19) were found to influence both, proliferation and cellular productivity. Increasing one or more of these miRNAs provides an easy and efficient approach for optimizing the production of a biomolecule. For introducing the miRNAs into the cell any of the methods mentioned herein or combinations thereof may be used.

In a further aspect, the invention relates to the use of a combination of at least one miRNA selected from group 1, at least one miRNA selected from group 2 and/or at least one miRNA-inhibitor inhibiting a miRNA selected from group 3, and at least one miRNA selected from group 4 or 5 and/or at least one miRNA-inhibitor inhibiting a miRNA selected from group 4 or 5, in producing a biomolecule in a cell. A combination of a miRNA promoting cellular production, a miRNA or miRNA-inhibitor suppressing cell death and/or a miRNA or miRNA-inhibitor regulating cell proliferation may be provided in various forms. For example, the miRNAs/inhibitors may be provided as a single nucleic acid construct. Alternatively, a multitude of nucleic acid molecules each encoding for a subset of miRNAs may be provided e.g. one expression vector encoding for at least one miRNA of group 1, a second expression vector encoding for a miRNA of group 2 and a third expression vector encoding for a miRNA-inhibitor directed against a miRNA of group 5. Likewise, the miRNAs and miRNA-inhibitors may be provided as a compilation of several pri- or pre-miRNA molecules or miRNA mimics. Thus, the miRNAs may be provided as a kit comprising the diverse miRNAs and/or inhibitors in a single composition or separately.

In a further aspect, the invention relates to a nucleic acid construct comprising a region encoding for at least one miRNA selected from the group consisting of SEQ ID NO.: 1-295 and/or a region encoding for at least one inhibitor directed against at least one miRNA selected from the group consisting of SEQ ID NO.: 296-609. All of the miRNAs of SEQ ID NO.: 1-295 were found to promote total biomolecule production, such that an increased amount of biomolecule could be harvested form cultures overexpressing any of these miRNAs. For most miRNAs, specific effects on distinct pathways (namely cell proliferation, cell death and cellular productivity) were found, which are supposed to account, at least partially, for the observed increase in overall biomolecule production. Accordingly, each of the miRNAs alone or in combination is suitable to enhance biomolecule production from a production cell. Additionally, some miRNAs appear to influence the cell's performance more generally, leading to an overall increase in volumetric production without significant alterations of cell survival, proliferation or cellular production. These miRNA were miR-721 (SEQ ID NO.: 157), miR-107-3p (SEQ ID NO.: 286), miR-181a-1-3p (SEQ ID NO.: 290) and miR-19b-2-5p (SEQ ID NO.: 292). It is suggested that these miRNAs instead of significantly altering one or two of said processes, rather influence all of them and possibly further cell signalling pathways. Similar to most of the miRNAs of SEQ ID NO.: 1-295 each of SEQ ID NO.: 296 to 609 were found to exert effects on cell proliferation and/or cell death. Inhibition of a single or a plurality of these miRNAs is suitable to promote cell survival and/or proliferation, leading to an increase in overall biomolecule production.

Accordingly, in a further aspect, the invention relates to a method for increasing the yield of a biomolecule produced by a cell cultured in vitro comprising the steps increasing the level of at least one miRNA selected from the group consisting of SEQ ID NO.: 1-295 in the cell, and/or decreasing the level of at least one miRNA selected from the group consisting of SEQ ID NO.: 296-609 in the cell.

Further more, the invention relates to a method for producing a biomolecule in a cell comprising the steps propagating the cell in a cell culture, increasing the level of at least one miRNA selected from the group consisting of SEQ ID NO.: 1-295 in the cell, and/or decreasing the level of at least one miRNA selected from the group consisting of SEQ ID NO.: 296-609 in the cell, and isolating the biomolecule from the cell culture.

In a preferred embodiment, wherein the miRNA and/or miRNA-inhibitor is added to the cell culture by electroporation or together with a transfectant, or is introduced to the cell culture by a viral vector.

In a further aspect, the invention relates to the use of at least one miRNA selected from group 1 for stimulating cellular production of a biomolecule produced by a cell cultured in vitro. The miRNAs of group 1 all showed a significant increase in cellular production, leading to an increase in the amount of biomolecule that was produced by the entire culture.

In a further aspect, the invention relates to the use of at least one miRNA selected from group 2 and/or a miRNA-inhibitor directed against a miRNA of group 3 for suppressing cell death of a cell cultured in vitro. By overexpressing any of the miRNAs of group 2 and/or inhibiting any of the miRNAs of group 3, cell survival is promoted, increasing the total number of production cells. This in turn results in an increased amount of total biomolecule produced.

In a further aspect, the invention relates to the use of at least one miRNA selected from group 4 or 5 and/or a miRNA-inhibitor directed against a miRNA of group 4 or 5 for regulating proliferation of a cell cultured in vitro. Cell proliferation may be specifically regulated depending on the state of the culture. At the beginning of the culture, proliferation may be enhanced to reach an optimal cell density as fast as possible. This may be achieved by overexpressing any of the miRNAs of group 4 and/or inhibiting any of the miRNAs of group 5. In contrast, once the culture is fully established, a reduction of proliferation may be advantageous to provide more capacity to the production of the biomolecule. This may be achieved by overexpressing any of the miRNAs of group 5 and/or by inhibiting any of the miRNAs of group 4.

### Examples

### Materials and Methods

### Cell Culture

### Culturing CHO Cells

Suspension-adapted CHO-SEAP cells, established from CHO DG44 cells (Life Technologies, Carlsbad, Calif., USA), were grown in TubeSpin® bioreactor 50 tubes (TPP, Trasadingen, Switzerland) in ProCHO5 culture medium (Lonza, Vervier, Belgium), supplemented with 4 mM L-Glutamine (Lonza) and 0.1% anti-clumping agent (Life Technologies). Culture medium for stable miRNA overexpressing CHO-SEAP cells was additionally supplemented with 10 μg/mL puromycin-dihydrochloride (InvivoGen, San Diego, Calif., USA). Generally, cell concentration of the pre-cultures was adjusted to 0.5×10⁶ viable cells per ml one day prior to transfection to ensure exponential growth and the cells were maintained at 37° C., 5% CO₂ and 85% humidity with agitation at 140 rpm (25 mm orbit) in an orbital shaker incubator (Sartorius Stedim, Goettingen, Germany or Kuehner, Birsfelden, Switzerland).

### Culturing Human Cell Lines

T98G, HCT116, SKOV3 and SGBS were grown in Dulbecco's Modified Eagle's Medium (DMEM) High Glucose, containing 4 mM glutamine, 100 μM pyruvate and 10% v/v fetal bovine serum (FBS) in T25, T75, T175 tissue culture flasks or 96 well tissue culture plates. Cells were maintained at 37° C., 5% CO₂ and 95% humidity.

### Cell Culture of HeLa Cells

Adherently growing HeLa DJ cells (MediGene AG, Planegg/Martinsried, Germany) were grown in high glucose Dulbecco's Modified Eagle Medium (DMEM) (Life technologies, Carlsbad, Calif., USA) supplemented with 10% heat-inactivated FBS (Sigma Aldrich, St. Louis, Mo., USA) and 2 mM GlutaMAX® (Life technologies). Cells were cultured in T75 or T175 tissue culture flasks and maintained at 37° C., 5% CO₂ and 95% humidity.

### Transfection of CHO Suspension Cell Lines

Non-viral delivery of miRNA mimics or small interfering RNAs (siRNAs) was performed using ScreenFect® A (InCella, Eggenstein-Leopoldshafen, Germany). Small scale transfections for the primary and secondary screening were conducted in U-bottom shaped 96-well suspension culture plates (Greiner, Frickenhausen, Germany). For secondary screening, selected miRNA mimics were transfected again and plates were placed on a Mini-Orbital digital shaker (Bellco, Vineland, USA) located inside a Heraeus® BBD 6220 cell culture incubator (Thermo Scientific) at 37° C., 5% CO₂, 90% humidity and agitation at 800 rpm. Scaled up transfections for target validation were carried out in 12-well suspension culture plates (Greiner) and plates were incubated in an orbital shaker incubator with agitation at 140 rpm. An entire murine miRNA mimics library (based on Sanger miRBase release 18.0) comprising 1139 different miRNA mimics (Qiagen, Hilden, Germany) was used for transfection and all transfections were done in biological triplicates. An Alexa Fluor®647 labeled non-targeting siRNA (AF647-siRNA) (Qiagen) was co-transfected with each effector and control miRNA as indicator of transfection efficiency. As functional transfection controls, an anti-SEAP siRNA (Qiagen), a CHO-specific anti-proliferative (used for the primary screen) as well as a cell death control siRNA (secondary screen) were used. A non-targeting, scrambled miRNA (Qiagen) was used as negative control (miR-NT). For plasmid DNA (pDNA) transfections, CHO-SEAP cells were nucleofected employing the NEON® transfection system (Life Technologies). 1.0×10⁷ viable cells were pelleted and resuspended in 110 μL of Buffer R (Life Technologies) followed by the addition of 25 μg endotoxin-free pDNA. Cells were nucleofected with one pulse at 1650 volts for 20 milliseconds and seeded in 10 mL of fresh culture medium. Transfected cells were subjected to antibiotic selection pressure 48 h post transfection by adding 10 μg/mL of puromycin-dihydrochloride to the cultures.

### Transfection of Adherent Cell Lines

Cells were seeded at 7.500/cm² (T98G), 10.000/cm² (HCT116) 13.000/cm² (SKOV3) or 6.000/cm² (SGBS) in 96 well tissue culture plates and grown for 24 h. At the day of transfection, transfection complexes were formed by combining 0.4 μl ScreenFectA, 4.6 μl Dilution Buffer, 5.0 μl miRNA (1 μM) and 90 μl DMEM and lipoplex formation was allowed for 20 min at room temperature. Culture medium was removed followed by addition of 100 μl of transfection complexes to each well. After 6 h another 75 μl of DMEM were added.

Transfection of miRNAs and Production of Recombinant Adeno-Associated Vectors (rAAVs)

One day prior to transfection, HeLa DJ cells (MediGene AG) were seeded in 12-well microplates at a cell density of 3.0×10⁴ cell per cm² in high glucose DMEM supplemented with 10% heat-inactivated FBS and 2 mM GlutaMAX®. On the day of transfection, cells were co-transfected with rAAV production plasmids and miRNA mimics using Lipofectamine™ 2000 (Life technologies). For each well, 1.8 μL of Lipofectamine™ 2000 was pre-diluted in 100 μL DMEM medium (Life Technologies). 1.5 μg of plasmid DNA comprising rAAV vector, HAdV helper plasmid (E2A, E4, VARNA 1 and 2) and HAdV5 E1 helper plasmid were mixed at a molar ratio of 1:1:1 in 100 μL DMEM medium, followed by the addition of 50 nM miRNA mimics (Qiagen, Hilden, Germany). Lipoplexes were allowed to form by combining diluted Lipofectamine™ 2000 with DNA/miRNA solutions followed by an incubation for 15 min at room temperature. Culture medium was removed and 800 μL of high glucose DMEM supplemented with 10% heat-inactivated FBS and 2 mM GlutaMAX® was added to each well. Finally, 200 μL of lipoplex solution were added sequentially to each well.

Cloning of miRNA Expression Vectors

Native miRNA precursor (pre-miR) sequences of Cricetulus griseus (cgr) cgr-MIR30a, cgr-MIR30c-1, and cgr-MIR30e were obtained by polymerase chain reaction (PCR) from hamster genomic DNA (gDNA). Therefore, gDNA was isolated from CHO-SEAP cultures. PCR from gDNA was performed using a 1:1 mixture of two different DNA polymerases from Thermus aquaticus (Taq) and Pyrococcus furiosus (Pfu) (Fisher Scientific, St. Leon Rot, Germany). The following PCR primers were used to amplify pre-miR sequences including approximately 100 bp of upstream and downstream genomic flanking regions: cgr-MIR30a (332 bp PCR fragment length), forward 5′-TTGGATCCAGGGCCTGTATGTGTGAATGA-3′ (SEQ ID NO.: 610), reverse 5′-TTTTGCTAGCACACTTGTGCTTAGAAGTTGC-3′ (SEQ ID NO.: 611), cgr-MIR30c-1 (344 bp PCR fragment length), forward 5′-TTGGATCCAAAATTACTCAGCCC-ATGTAGTTG-3′ (SEQ ID NO.: 612), reverse 5′-TTTTGCTAGCTTAGCCAGAGAAGTG-CAACC-3′ (SEQ ID NO.: 613); cgr-MIR30e (337 bp PCR fragment length), forward 5′-TTGGATCCATGTGTCGGAGAAGTGGTCATC-3′ (SEQ ID NO.: 614), reverse 5′-TTTTGCTAGCCTCCAAAGGAAGAGAGGCAGTT-3′ (SEQ ID NO.: 615). Amplified PCR products contained BamHI/NheI restriction sites at their respective ends which were introduced by the PCR primers. Digested PCR fragments were ligated into a miRNASelect™ pEGP-miR expression vector (Cell Biolabs, San Diego, Calif., USA) between BamHI and NheI restriction sites employing the Rapid DNA Dephos & Ligation Kit (Roche Diagnostics). The correct integration of the pre-miR sequences was confirmed for all miRNAs by DNA sequencing (SRD, Bad Homburg, Germany). The miRNASelect™ pEGP-miR-Null vector (Cell Biolabs) which lacks any pre-miR sequence served as negative control.

### Quantitative Flow Cytometry

For cellular analysis, transfected CHO-SEAP cells were analyzed for cell concentration, viability, necrosis and transfection efficiency 72 h post transfection. Cells were analyzed by high-throughput quantitative flow cytometry employing a MACSQuant® Analyzer (Miltenyi Biotech, Bergisch-Gladbach, Germany) equipped with a violet (405 nm), blue (488 nm) and red (635 nm) excitation laser. 40 μL of a 3× staining solution [ProCHO5 medium (Lonza) supplemented with 15 μg/mL propidium iodide (PI) (Roth, Karlsruhe, Germany), 6 μg/mL Calcein-Violet450-AM (eBioscience, Frankfurt, Germany), 0.5 mM EDTA] were added to 80 μL of cell suspension and incubated for 20 min at 37° C., 5% CO₂ and 85% humidity. Subsequently, cells were counted and viability was measured by means of Calcein-Violet450-AM staining. Necrotic/late apoptotic cells were detected by PI exclusion and transfection efficiency was determined by analyzing viable cells for Alexa Fluor®647 fluorescence.

### Analysis of Apoptosis

Transfected cells were analyzed for apoptosis using a Nicoletti staining procedure. To this end, adherently growing cells were washed with PBS and detached by the addition of 35 μl trypsin/EDTA-solution and reaction was stopped by the addition of 80 μl DMEM containing 10% v/v FBS. Complete material including the washing step was subjected to the analysis. Suspension cells were directly employed to the staining procedure. Cells with a DNA content less than 2n (=Sub-G₀/G₁ cells) were classified as apoptotic. Hence, 50 μL of transfected cell suspension was transferred to a new 96-well microplate containing 100 μL of culture medium per well and centrifuged for 5 min at 150×g. 100 μL of supernatant was transferred to another 96-well microplate used for SEAP protein quantification. The cell pellet was resuspended in 100 μL of Nicoletti staining solution (1× Phosphate buffered saline (PBS) supplemented with 0.1% sodium citrate, 0.05% Triton X-100, 10 μg/mL PI and 1 U/μL RNase A) and incubated in the dark for 30 min at 4° C. Treated cells were analyzed by quantitative flow cytometry on a MACSQuant® Analyzer (Miltenyi Biotech).

### SEAP Quantification

SEAP protein levels in the culture supernatant of transfected cells were quantified in white 96-well non-binding microplates using a SEAP reporter gene assay (Roche Diagnostics). In principle, the chemiluminescent substrate CSPD (3-(4-methoxyspiro[1,2-dioxetane-3,2′(5′-chloro)-tricyclo(3.3.1.1^{3.7})decane]-4-yl)phenylphosphate) is dephosphorylated by SEAP, resulting in an unstable dioxetane anion that decomposes and emits light at a maximum wavelength of 477 nm. Endogenous alkaline phosphatases were inhibited by incubating the samples for 30 min at 65° C. following a chemical inactivation using a provided Inactivation Buffer (Roche Diagnostics). Due to high SEAP expression levels of the CHO-SEAP cell line, culture supernatants were pre-diluted 1:60 in fresh culture medium. Chemiluminescence was detected after addition of CSPD substrate using a SpectraMax® M5e microplate reader (Molecular Devices, Sunnyvale, Calif., USA).

qRT-PCR Analysis

Total RNA (including small RNAs <200 bp) was isolated using the miRNeasy mini Kit (Qiagen) according to the protocol provided by the manufacturer. RNA concentration and purity was determined by UV-spectrometry using a NanoDrop® spectrophotometer (Thermo Scientific). Complementary DNA (cDNA) was synthesized from 1 μg total RNA using the miScript II RT Kit (Qiagen). RT-PCR was performed with 20⁻¹ diluted cDNA using the miScript SYBR green PCR kit (Qiagen) for detection of mature miRNAs on a LightCycler® 480 (Roche Diagnostics). The following miRNA-specific primers were used: mature miR-30a-5p forward, 5′-TGTAAACATCCTCGACTGGAAGC-3′ (SEQ ID NO.: 616); miR-30b-5p forward, 5′-TGTAAACATCCTACACTCAGCT-3′ (SEQ ID NO.: 617); miR-30c-5p forward, 5′-TGTAAACATCCTACACTCTCAGC-3′ (SEQ ID NO.: 618); miR-30d-5p forward, 5′-CTTTCAGTCAGATGTTTGCTGC-3′ (SEQ ID NO.: 619); miR-30e-5p forward, 5′-TGTAAACATCCTTGACTGGAAGC-3′ (SEQ ID NO.: 620); the miScript Universal Primer (Qiagen) served as reverse primer for each mature miRNA; U6 forward, 5′-CTCGCTTCGGCAGCACA-3′ (SEQ ID NO.: 621); U6 reverse, 5′-AACGCTTCACGAATTTGCGT-3′ (SEQ ID NO.: 622). Relative mature miRNA expression differences were calculated by applying the comparative C(T) method.

rAAV Vector Quantification

72 h post transfection, HeLa DJ cells were subjected to three freeze/thaw cycles liquid nitrogen/37° C.) and cell debris was removed by centrifugation at 3700×g for 15 min. AAV genomic particles were determined by qRT-PCR based on quantification of AAV-2 inverted terminal repeats (ITRs). Pre-treatment of crude sample for removal of host cell and unpacked DNA was adapted from the procedure described by Mayginnes and colleagues (Mayginnes et al., 2006), with following changes: samples were diluted 200-fold in DNase reaction buffer (22.2 mM Tris/HCl pH 8.0, 2.2 mM MgCl2) before DNasel treatment (Qiagen) and final sample was further diluted 3-fold in MilliQ H₂O. Buffer controls containing 1×10⁶ AAV vector plasmid copies and/or DNase I were treated equally. The reactions were performed on a CFX96™ instrument (Bio-Rad Laboratories Inc., Hercules, Canada) in a total volume of 25 μL, including 12.5 μL SYBR Green Master Mix (QIAGEN), 2.5 μL AAV2 ITR primer mix (Aurnhammer et al., 2012), 5 μL water and 5 μL template (pre-treated samples/controls, water for non-template control or serial dilution of standard from 10² to 10⁸ plasmid copies). PCR conditions were as follows: pre-incubation at 95° C. for 5 min, followed by 39 cycles of denaturation at 95° C. for 10 s and annealing/extension at 60° C. for 30 s. Data analysis occurred using CFX Manager software (Bio-Rad Laboratories Inc.).

### Results

Transient High-Content miRNA Screen in Recombinant CHO-SEAP Suspension Cells

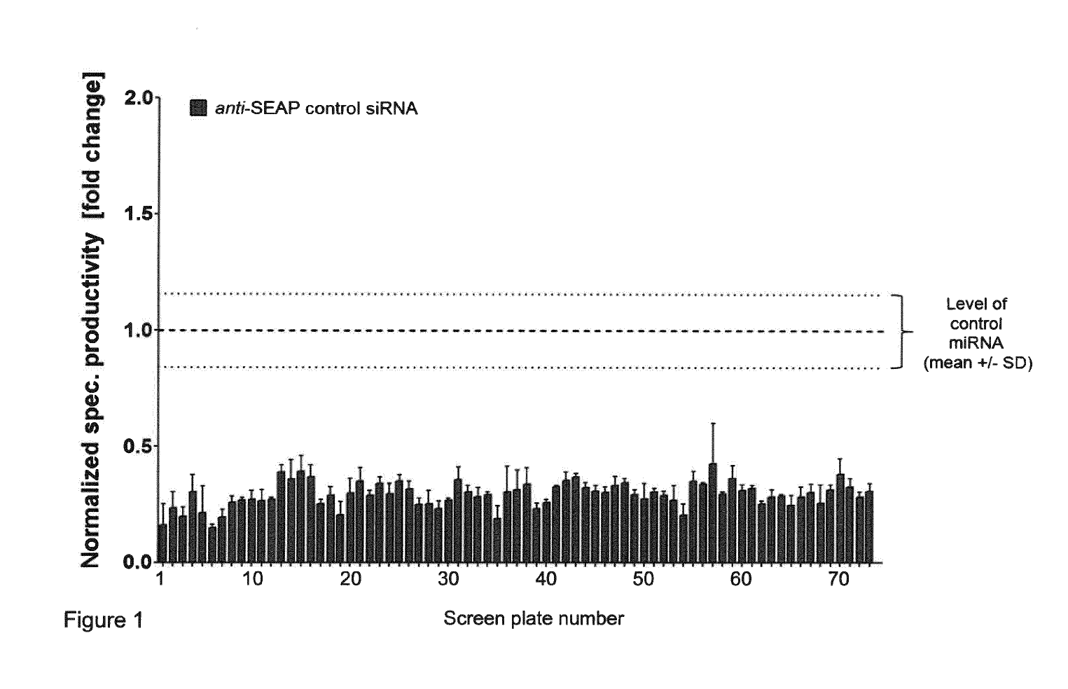

The inventors performed a high-content microRNA screen using 1139 different miRNA mimics in a recombinant CHO-SEAP suspension cell line to identify miRNAs improving cellular function. In this conjunction, all transfected cells were analyzed for various cellular parameters employing a multiparametric flow cytometry-based cell analysis. Transfection conditions for small double-stranded RNAs in 96-well format were carefully optimized and several functional controls were used which included a non-targeting control miRNA (miR-NT), a siRNA against the SEAP mRNA (anti-SEAP siRNA) as well as a CHO-specific anti-proliferative siRNA. Using a novel non-viral transfection reagent (ScreenFect® A) which has previously been demonstrated to efficiently and functionally deliver miRNA mimics into cells grown in a complex production medium (Fischer et al., 2013), high transfection rates of >95% could be reproducibly achieved at low cytotoxicity rates. As delivery control, a fluorescently-labelled non-targeting siRNA (AlexaFluor®647-siRNA) was co-transfected with all effector and control miRNAs/siRNAs, respectively. Cell concentration, viability, necrosis and transfection efficiency was measured 72 h post transfection by high-throughput quantitative flow cytometry. Analysis of apoptotic cell death by means of Nicoletti staining was also performed on a quantitative flow cytometer. SEAP protein concentrations were determined using a commercially available SEAP reporter assay. A cultivation period of three days was chosen to account for both the time-limited transient effects of miRNA mimics and the manifestation of changes in cell phenotype. A significant decrease in SEAP productivity of cells transfected with an anti-SEAP siRNA as well as significant decrease in the viable cell density (VCD) of cells transfected with an anti-proliferative siRNA was indicative for functional transfections in all screen plates (FIG. 1). In addition, spiked-in AlexaFluor®(AF) 647-siRNA confirmed uptake of miRNA mimics in each well.

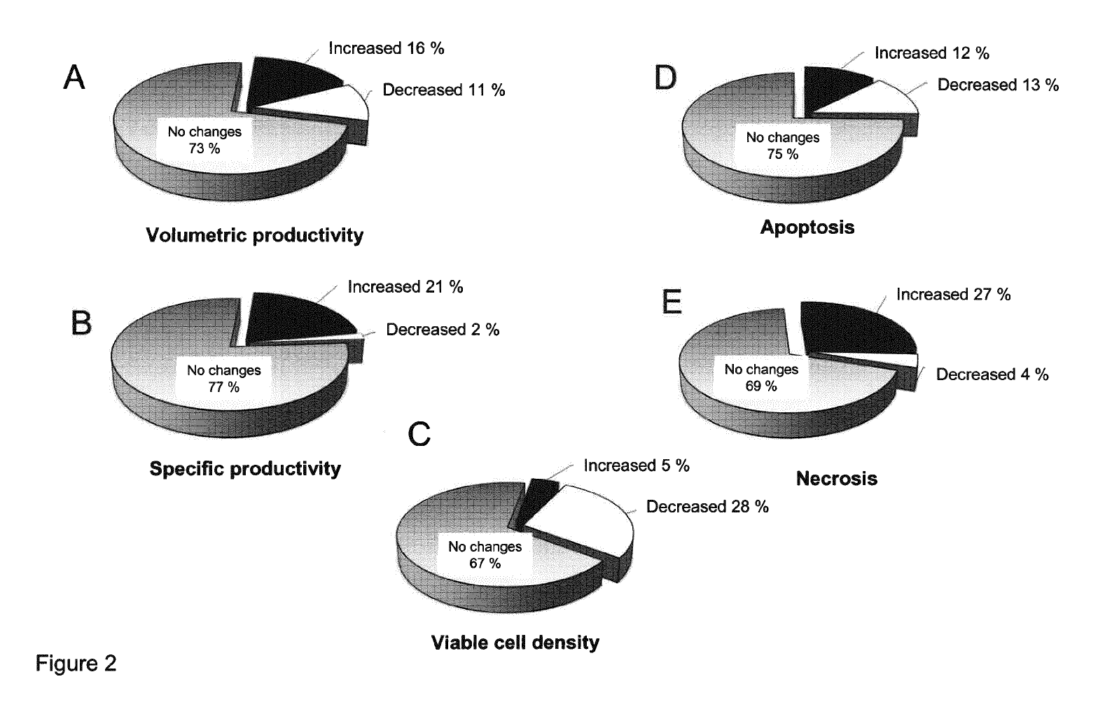

Data normalization was performed to allow for inter-plate comparisons by normalizing each sample value to the mean value of the respective on-plate control (miR-NT). Significant changes on each readout parameter were determined by applying a one-way analysis of variances (ANOVA) combined with a Dunnett's multiple comparison test (against the on-plate miR-NT control; p<0.05). The normalized mean values for all 1139 effector miRNAs considering important cell characteristics such as VCD, apoptosis, necrosis/late apoptosis, specific and volumetric SEAP productivity were determined. Cake charts indicating the number of statistically significant hits as percentage of all mimics tested are shown in FIG. 2. Regarding SEAP productivity, a large proportion of 16% of the transfected miRNAs significantly increased SEAP yields in the supernatant after 72 h with the best candidates showing an improvement of up to two-fold (FIG. 2A). Significantly elevated cell-specific productivity was even detected for 21% of the miRNAs (FIG. 2B). However, this was partly in conjunction with a decreased cell concentration without inducing cell death. In particular, of the 314 miRNAs which increased mean specific productivity by at least 20% were also found to decrease mean VCD by up to 69% three days post-transfection without lowering cell viability. Significantly higher viable cell densities were determined for 5% of all miRNAs, whereas 13% of all miRNAs decreased apoptosis rates (FIGS. 2C and D). The percentage of miRNAs boosting cell proliferation was in line with the fact that 4% of the miRNA library decreased the number of necrotic cells indicating higher viabilities following miRNA introduction (FIG. 2E).

Screen Analysis Identified Functionality of miR-30 Family

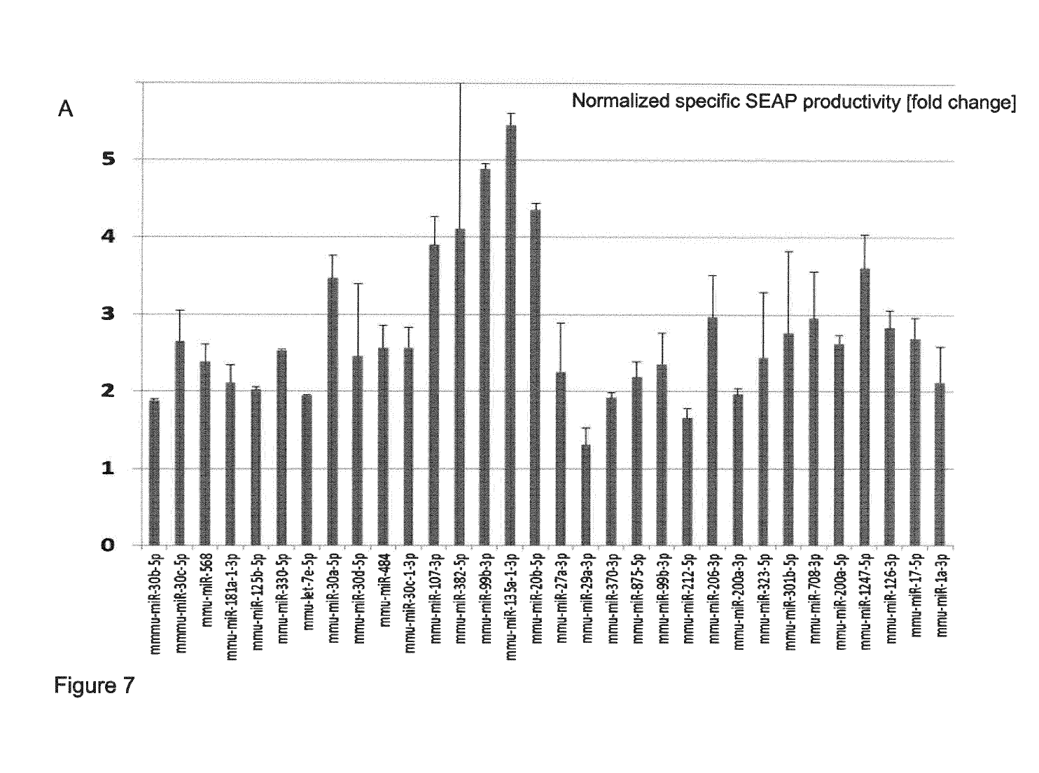

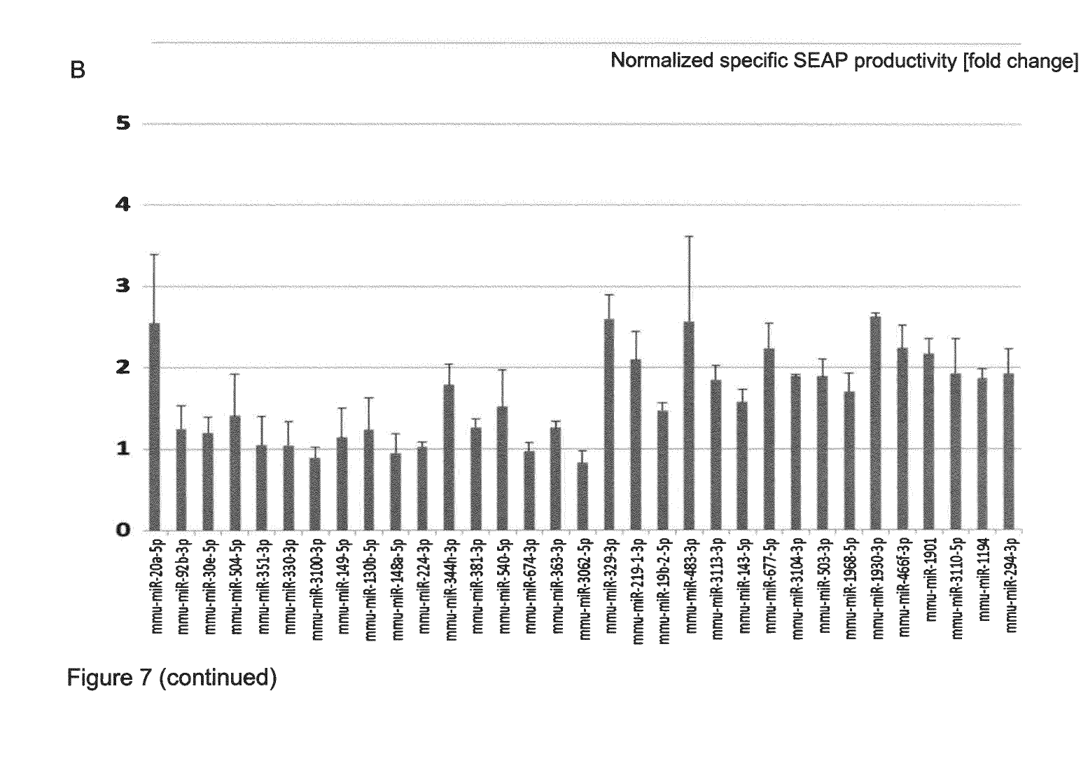

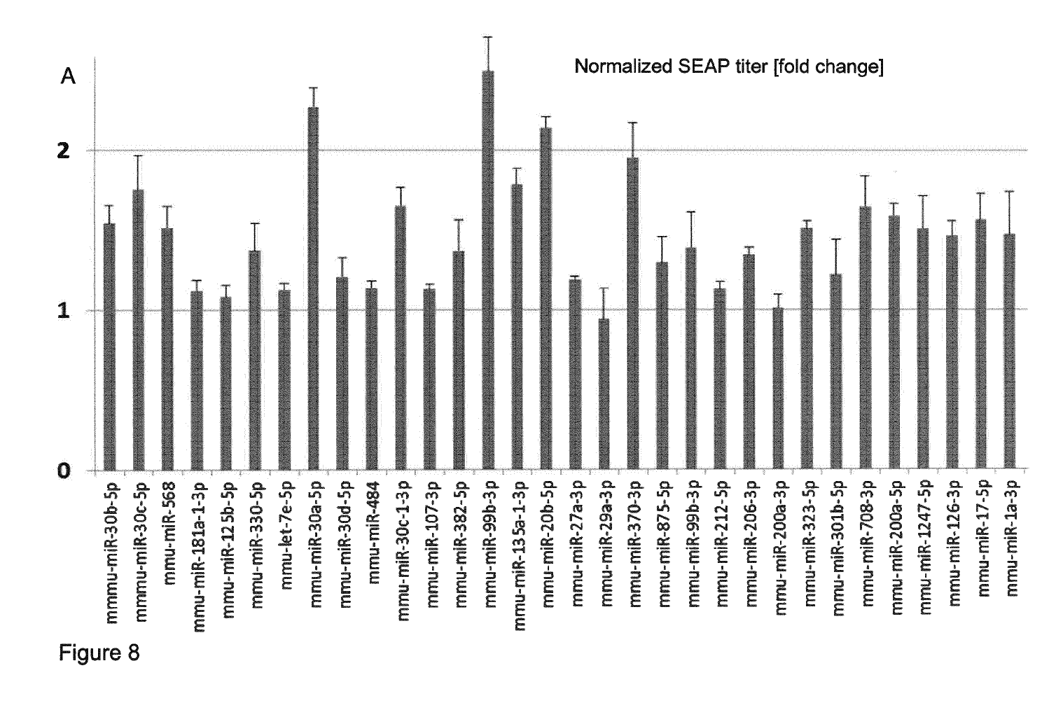

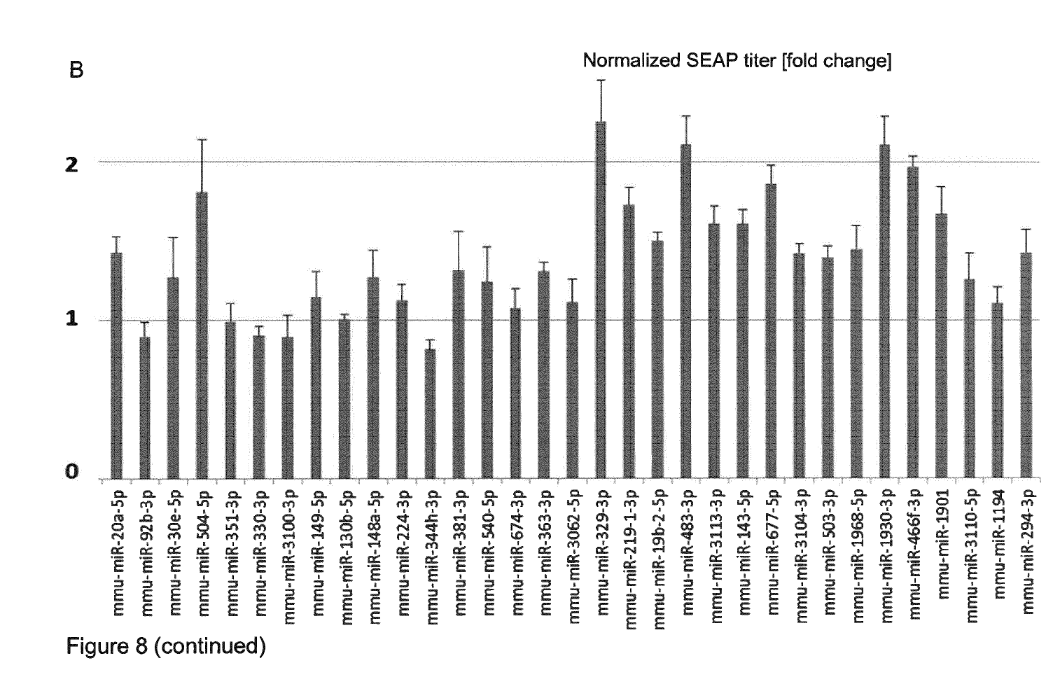

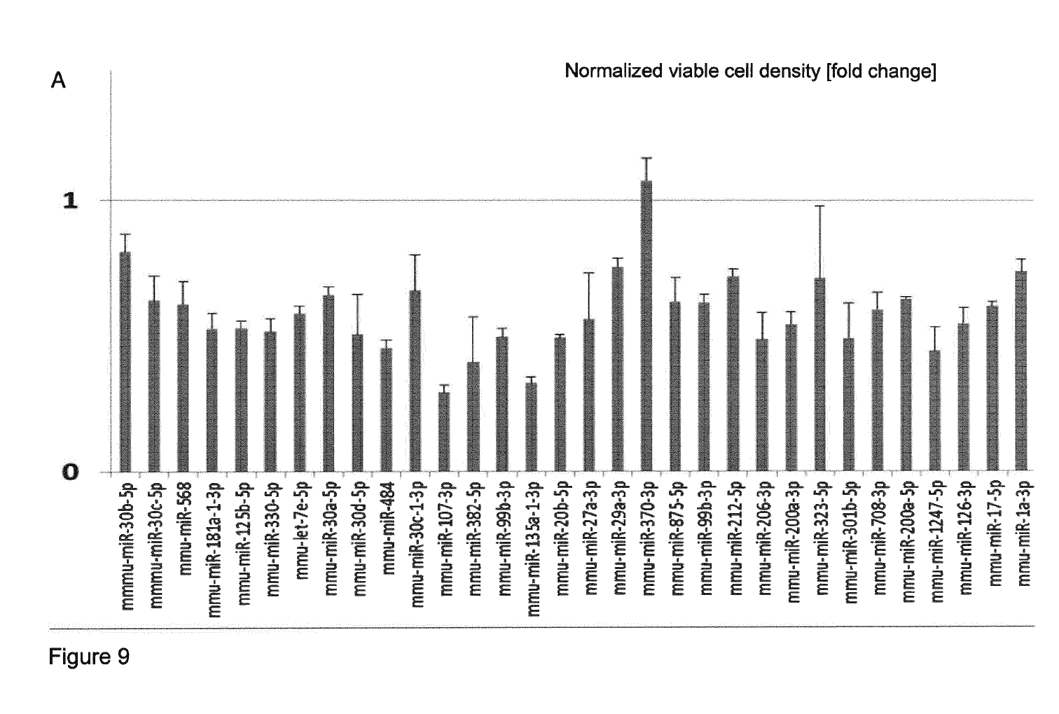

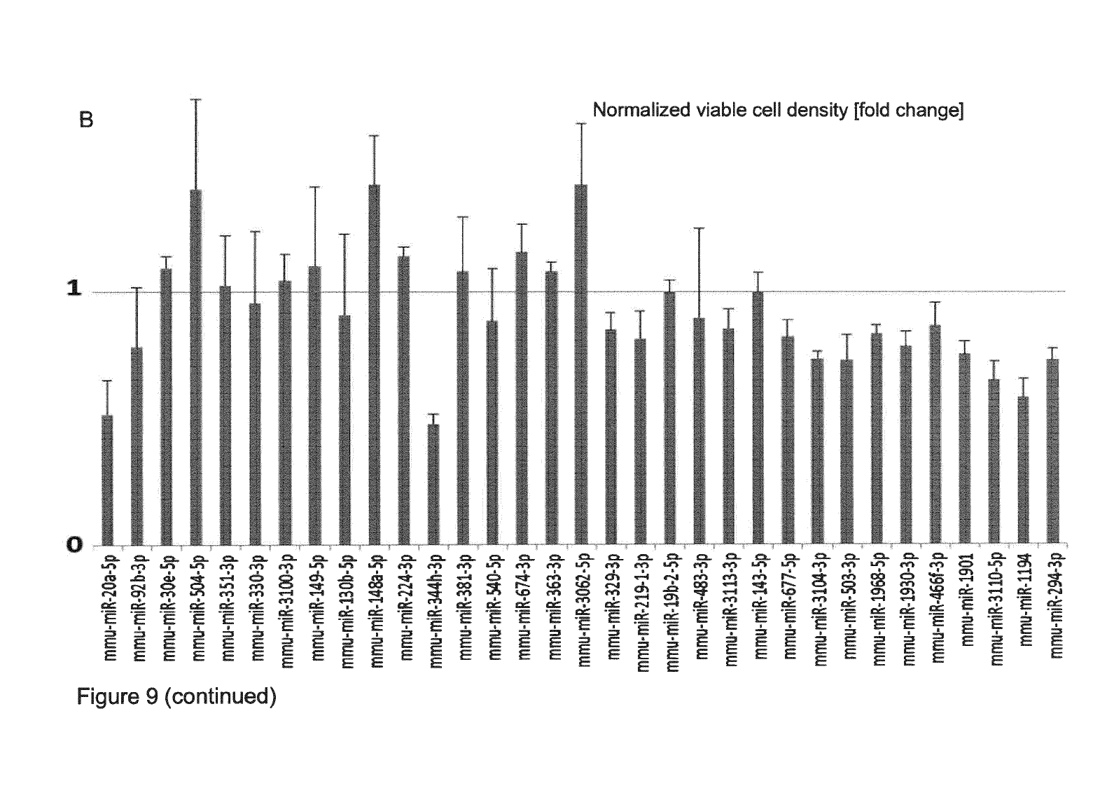

In order to validate the results obtained through the primary screen the inventors performed a secondary screen by transiently transfecting a subset of selected miRNA hits in agitated cultures. An agitated culture mode in multi-well plates is much more comparable to standard shaking flask cultivations, in which putative oxygen limitations of static cultures are substantially overcome. Shaking speed for 96-well plates together with the miRNA mimics concentration was carefully optimized to allow for robust cultivation and transient transfection in suspension. In a first step, 297 miRNA hits derived from the primary cellular screen were selected for a reassessment of their positive influence on at least one of the bioprocess relevant cellular parameters mentioned above. This approach confirmed phenotypic effects for most miRNAs as compared to the primary screen (FIGS. 7, 8 and 9), pointing towards a high reproducibility of the high-content screening method.

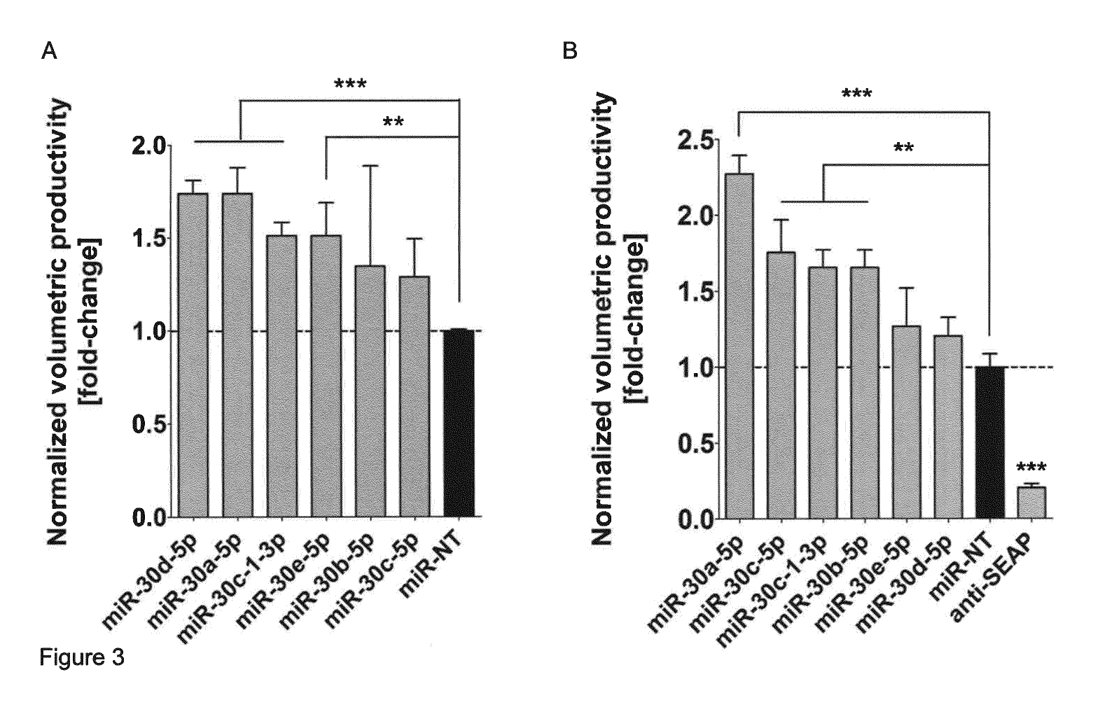

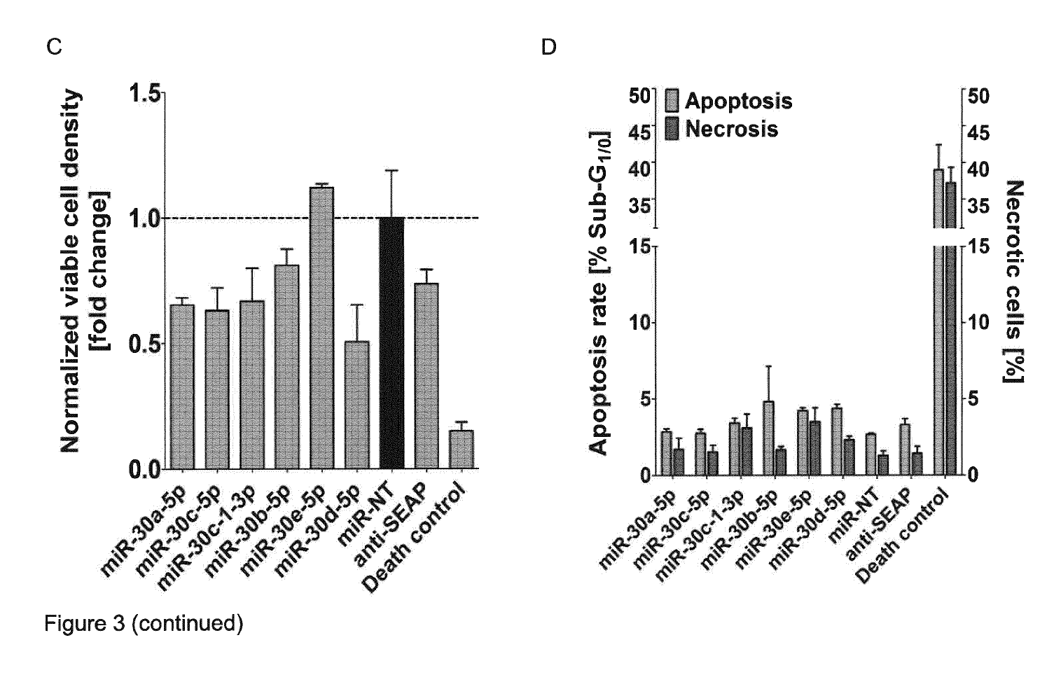

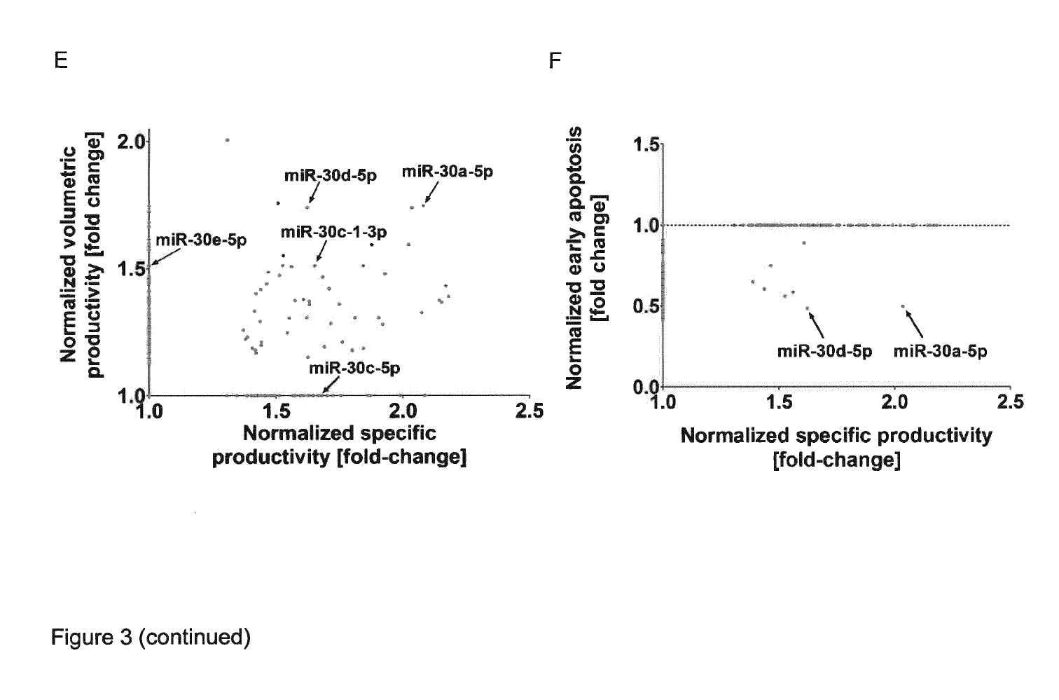

By analyzing the results of the both screens, the entire miR-30 family (comprising miR-30a, miR-30b, miR-30c, miR-30d, miR-30e) clearly contributed towards an enhanced SEAP production in CHO cells. In all miR-30 members the mature 5p-strands considered to be the guide strand induced the observed cellular phenotypes. However, miR-30c-1-3p, as the only 3p strand among the miR-30 family, was also found to substantially elevate SEAP productivity. FIG. 3A shows the respective fold changes in volumetric SEAP yield for all six productivity-improving miR-30 family members in the primary screen. Although the increase mediated by miR-30b-5p was not statistically significant due to high standard deviation of the biological triplicates, the inventors included it into the graph as for its obvious tendency contributing towards a higher volumetric productivity.

In addition, the miR-30 family could be reliably confirmed as potent driver of recombinant protein expression in CHO cells in the validation screen (FIG. 3B). By transfection of larger proportions of miRNA mimics (50 nM), compared to transfections in static cultures (15 nM), the increase in SEAP productivity was even more pronounced without an induction of concentration dependent off-target effects. The marked increase in SEAP production was accompanied by decreased cell densities in miR-30 transfected cultures (FIG. 3C). However, viability was not negatively affected (FIG. 3D) which promotes the assumption that the cells used most of their energetic resources for the substantially enhanced protein production rather than for cell growth and proliferation.

A characteristic feature of a miRNA family is that the mature miRNA strands share a common miRNA ‘seed’ sequence that are perfectly base paired with their mRNA targets. Besides a common ‘seed’ composing 7 nucleotides at the 5′ end of all miR-30-5ps, they also share the nucleotides at positions 9 to 11 (UCC), and 15 to 17 (ACU), respectively. Considering an overall length of 22-23 nucleotides for miR-30, this finding suggests that this miRNA family share a minimum of 60% sequence similarity, while miR-30a-5p, miR-30d-5p and miR-30e-5p even share >90% sequence homology.

To gain insights into multiple effects of respective miRNAs, one cellular parameter can be plotted against another, enabling the identification of highly interesting functional candidate miRNAs for cell engineering. Towards this end, phenotypic changes beneficial for bioprocess performance, such as an increase in protein production and viable cell density, or a decrease in apoptosis were investigated. A detailed analysis of the miR-30 family revealed that the three miRNAs miR-30a-5p, miR-30c-1-3p and miR-30d-5p exhibited combined effects in both increasing volumetric and specific productivity (FIG. 3E), and miR-30a-5p and miR-30d-5p both additionally decreased the number of apoptotic cells highlighting their potential as attractive targets for cell engineering (FIG. 3F).

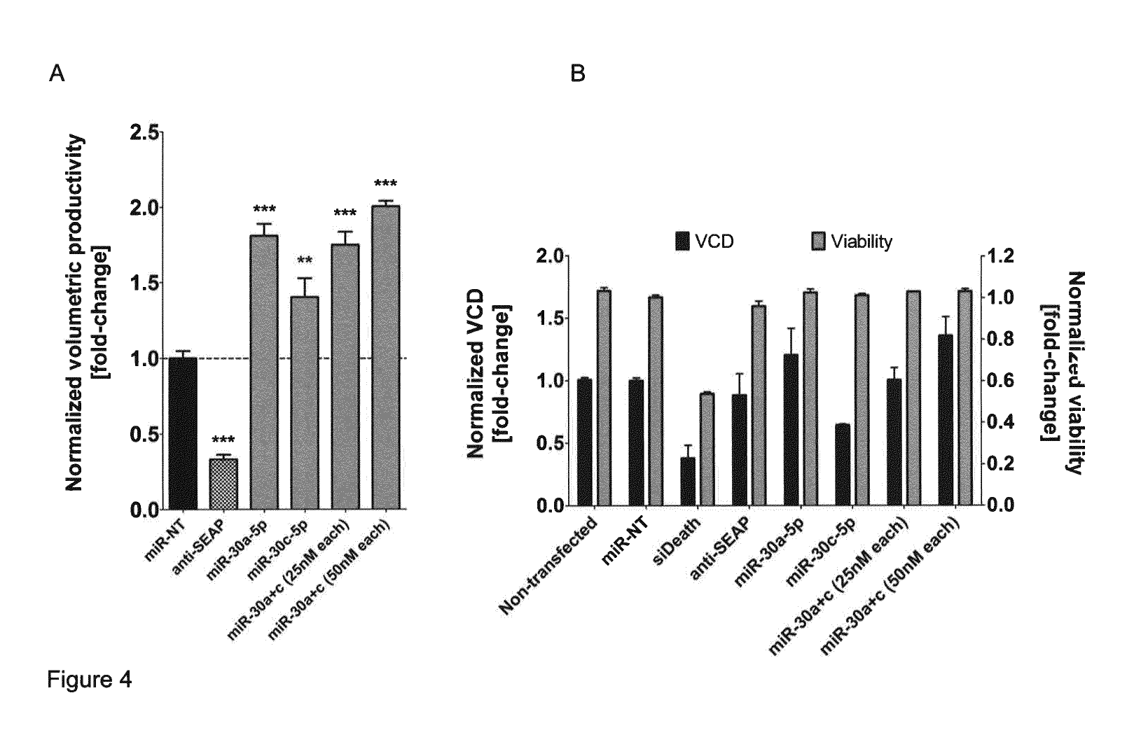

To further examine the potential of the miR-30 family to enhance protein production in CHO cells, the inventors selected two miR-30 family members exhibiting various extent of recombinant SEAP production increase (miR-30a-5p and miR-30c-5p) and performed a scale-up experiment by transfecting these miRNAs separately as well as combinations of both miRNAs in elevated culture scale. Similarly, miR-30a-5p and miR-30c-5p substantially increased volumetric and specific SEAP productivity after transient transfection in 2 mL batch cultures (FIG. 4A). CHO-SEAP cultures transfected with miR-30a-5p alone showed higher cell densities after 72 h, while introduction of miR-30c-5p resulted in decreased cell density (FIG. 4B). However, viability was not negatively affected suggesting that reduced cell densities might be due to a substantial increase in cell-specific SEAP productivity. Co-transfection of both miRNA species in equal concentrations (25 nM each) could reverse the growth-inhibiting effect of miR-30c-5p and resulted in higher SEAP titers as compared to cells transfected with 50 nM of miR-30c-5p mimics. Moreover, by increasing miR-30a/miR-30c concentrations up to 50 nM (100 nM total miRNA concentration), viable cell density was further increased compared to miR-NT transfected control cells, and exhibited values similar to cells transfected with 50 nM miR-30a-5p mimics. This might argue for additive or even synergistic effects of miR-30a and miR-30c, which would have important implications for a combined stable expression of various miRNAs.

Stable Overexpression of miR-30 Family Members

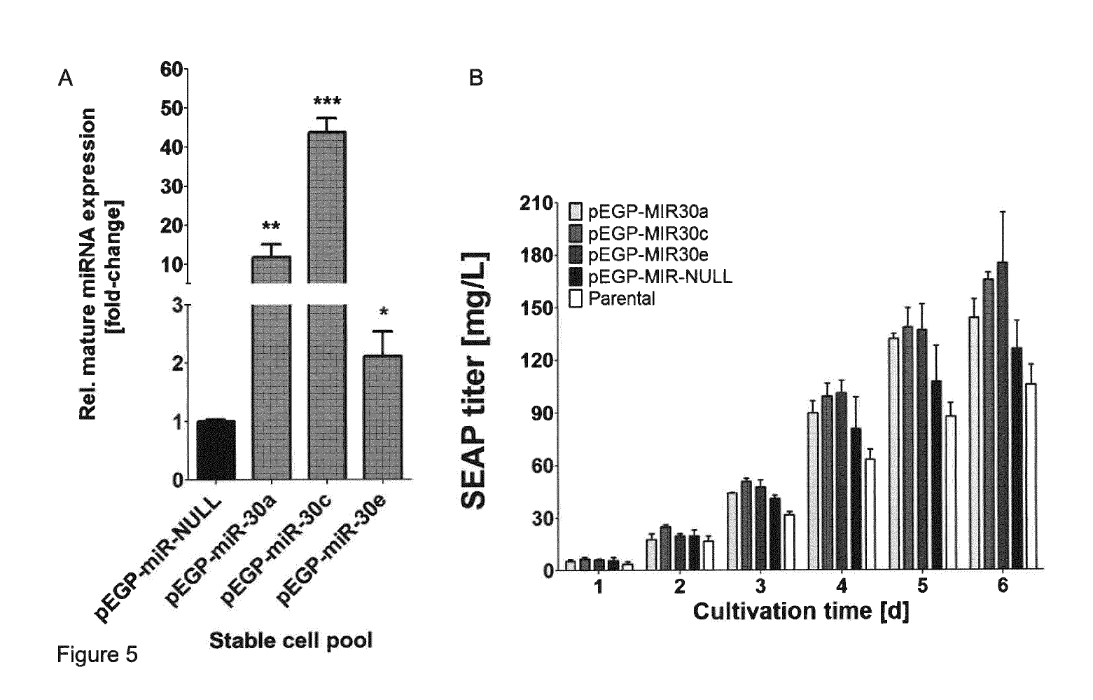

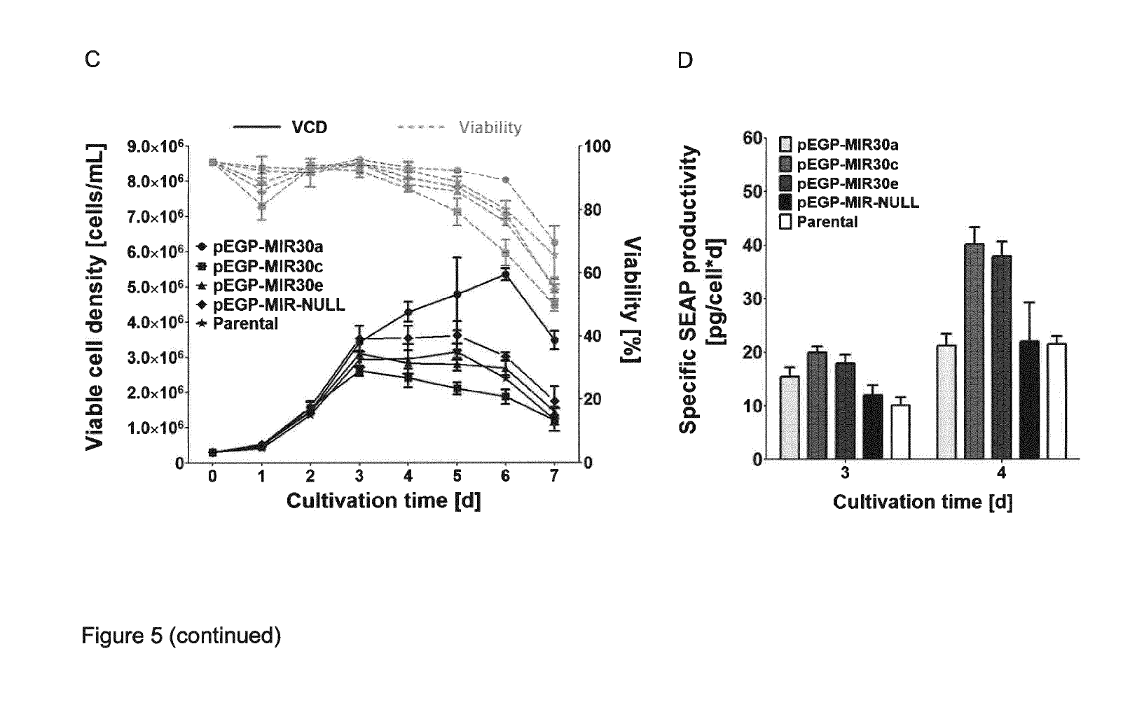

To confirm that results of transient introduction of miRNA mimics can be interpolated to stable miRNA overexpression, the inventors selected three miR-30 family members and established stable overexpressing cell pools based on the CHO-SEAP parental cell line. For stable long-term expression of target miRNAs the respective precursor sequences have to be integrated into the host cell genome. A correct intranuclear Drosha/DGCR8 processing requires the native genomic sequence context of endogenous pre-miRs including appropriate upstream and downstream flanking regions. The inventors have therefore PCR-amplified the endogenous precursor miRNA sequences of MIR30a, MIR30c and MIR30e, including approximately 100 bp of both up- and downstream flanking regions from genomic DNA, and subcloned them into a mammalian expression vector. The pre-miR sequences were inserted upstream of a green fluorescent protein-puromycin (GFP-Puro) fusion protein under the control of the human elongation factor 1 alpha (EF1α) promoter (FIG. 5A). This feature offers two advantages: Firstly, it enables the detection of positively transfected cells via GFP-fluorescence (as well as facilitates fluorescent-activated cell sorting), and secondly, it allows for selection of stably transfected cells by adding antibiotic pressure to the cultures. Moreover, the EF1α promoter induces strong transgene as well as miRNA expression and has been reported to be less prone to epigenetic gene silencing in CHO cells compared to viral promoters such as the human cytomegalovirus (hCMV) immediate early promoter. As a result, long-term miRNA overexpression in recombinant CHO cells is expected to be more stable and efficient as compared to previously described miRNA expression approaches in which only the mature miRNA-5p and -3p strands were integrated into an artificially created chimeric stem-loop.

Stable cell pools overexpressing each member of the miR-30 family were successfully established by puromycin selection and overexpression of mature miRNAs was assessed via qRT-PCR (FIG. 5B). Notably, the fold-change value of miRNA overexpression is highly dependent on the endogenous level of the respective mature miRNA. qRT-PCR analysis revealed that miR-30e-5p is highly abundant in CHO cells as compared to miR-30a-5p or miR-30c-5p, which are only moderately expressed, possibly explaining the observed differences in miR-30 overexpression in the stable pools.

Stable MIR30a, MIR30c and MIR30e overexpressing pools were batch-cultivated for 7 days and compared to mock control cells (pEGP-MIR-Null) as well as the parental CHO-SEAP cell line. Analysis of SEAP protein concentration in the supernatant confirmed that CHO-pEGP-MIR30a, CHO-pEGP-MIR30c and CHO-pEGP-MIR30e produced significantly more SEAP as compared to control cells (FIG. 5C). To investigate if the observed increase in volumetric productivity was due to an increase in either cell number or specific productivity, the inventors have analyzed the cell density and viability and discovered that MIR30a overexpressing cells reached far higher cell density and viability from day 3 post-seeding as compared to parental CHO-SEAP cells (FIG. 5D). The accumulation of metabolic side products as well as a decrease in nutrient supply by depleted culture media is usually in conjunction with decreased proliferation rates as seen by the initiation of the stationary growth phase of negative control (pEGP-MIR-Null) and parental CHO-SEAP cells. The fact that MIR30a overexpressing cells kept growing at higher viabilities until day 6 post seeding, together with the observation that transient introduction of miR-30a-5p decreased apoptosis rate (FIG. 3F), points toward an anti-apoptotic function of MIR30a.

In contrast, MIR30c overexpressing pools showed slightly decreased cell concentrations whereas MIR30e overexpression had no significant effect on cell density and viability during batch cultivation. However, cell-specific SEAP productivity was substantially increased by almost two-fold in MIR30c and MIR30e overexpressing cells, respectively (FIG. 5E). In this conjunction, the extraordinarily enhanced recombinant protein productivity might be one possible reason for the diminished cell growth of the CHO-pEGP-MIR30c pool as well as for the earlier drop in viability, which might be due to a faster consumption of nutrients in the media. Moreover, the inventors observed that both miR-30c strands (5p and 3p), which are derived from the same pre-miR-30c precursor, enhanced recombinant protein expression in transient screenings (FIGS. 3A and B). Hence, another possible reason could be that since both strands are more abundant as a result of MIR30c overexpression, mature miR-30c-5p and miR-30c-1-3p act simultaneously leading to stronger phenotypic effects.

Strikingly, although these three miRNAs share the same seed sequence and the residual sequence only varies in a few nucleotides the effects on the cell phenotype is remarkably diverse. This illuminates that the specificity of a given miRNA to its target mRNAs and therefore its biological function is determined by the entire miRNA sequence rather than solely by the seed sequence. Another reason for the diverse function of the miR-30 family could be that since mature sequences are almost identical, it would be conceivable that miRNA precursor sequences play a critical role for miRNA fate and might be involved in determining function of the miRNA. Taken together the results of stable miR-30 overexpression finally proved that effects of transient miRNA mimics transfection experiments can be reproduced in a stable fashion, and more importantly, it highlights again that miRNAs are attractive tools for improving culture performance of biopharmaceutical production cells.

Mature miR-30 Expression Levels are Upregulated During Stationary Growth Phase

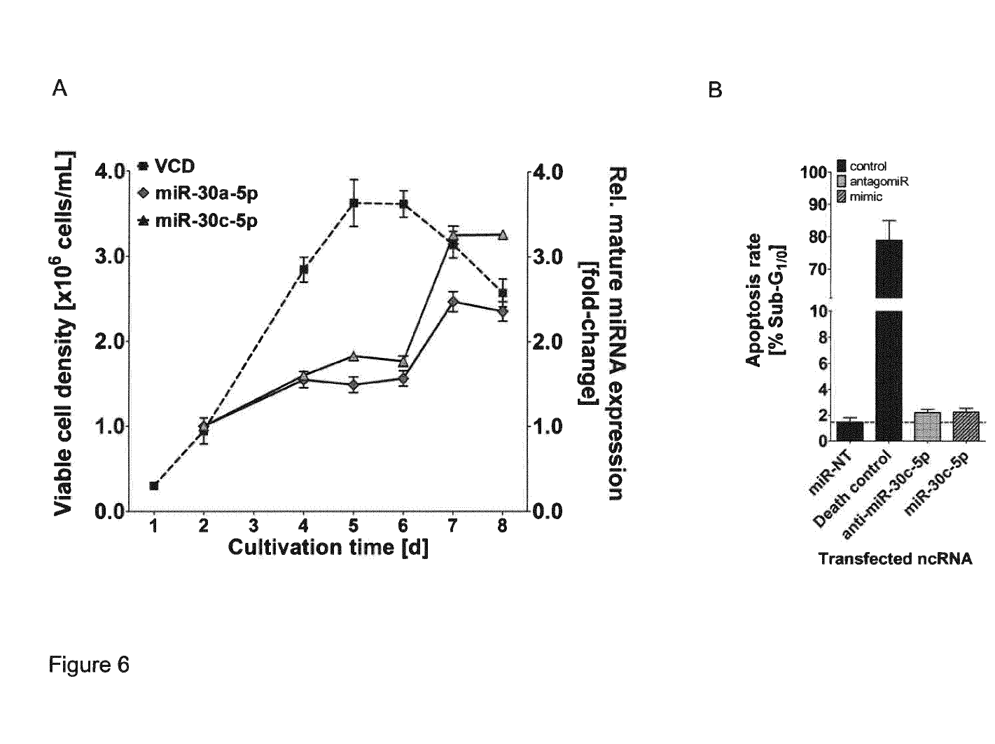

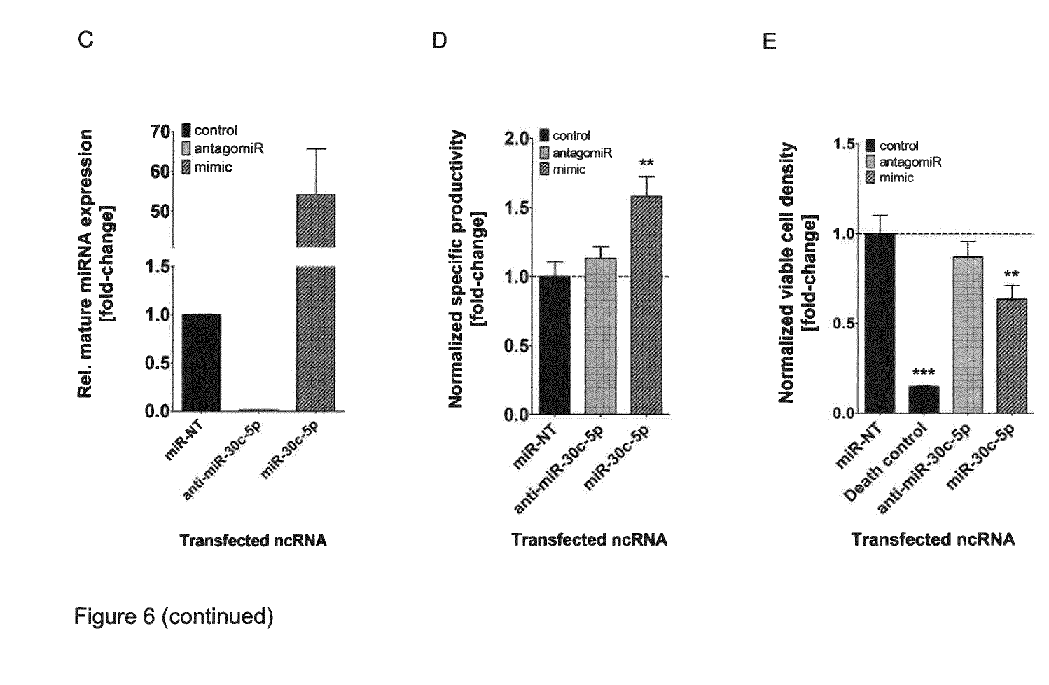

A batch suspension cell culture production process is generally divided into different phases with the stationary phase to be considered as the main production period where cells switch their metabolism from growth to increased protein expression, a feature which is exploited in fed-batch as well as in biphasic production processes. The miR-30 family has previously been demonstrated to be expressed by different CHO strains as well as under various culture conditions. The inventors hypothesized that if the miR-30 family actually contributes to increased protein production in CHO cells, the concentration of mature miR-30 molecules might be more abundant in the stationary phase than during exponential growth. To test this postulate the inventors performed three independent batch cultivations of CHO-SEAP cells, and analyzed expression levels of miR-30a-5p and miR-30c-5p, respectively, during the cultivation process. qRT-PCR analysis revealed that mature miR-30a and miR-30c were strongly upregulated during the stationary phase of a CHO batch culture (FIG. 6A). Although expression levels of both miRNAs still remained upregulated in the decline phase which is the last stage mainly driven by apoptotic cell death the miR-30 family may not be involved in apoptosis since at no times after transient (FIG. 6B) or stable miR-30 overexpression (FIG. 5D), increased apoptosis was observed. It is therefore rather likely that the cells might use miR-30 as endogenous vehicle to control the metabolic shift towards a more effective protein expression. Compared to proteins which have to be tediously translated, correctly folded as well as posttranslationally modified, microRNAs as small RNAs only have to be transcribed and processed to be readily available for gene regulation. This would promote the assumption of recent studies which classified miRNAs as smart endogenous tool to confer rapid transformation in cell phenotype.

To determine whether the observed effects after ectopic miR-30 overexpression could be reversed by knockdown of miR-30 family members, the inventors transiently repressed endogenous miR-30c-5p and subsequently examined the consequences on protein productivity and viable cell density of CHO-SEAP cells. Using antisense inhibitors specific for mature miR-30c-5p, so-called antagomiRs or miRNA-inhibitors, the inventors found that endogenous miR-30c expression was strongly attenuated (FIG. 6C). However, neither specific SEAP productivity nor viable cell density was significantly affected by anti-miR-30c-5p antagomiRs, suggesting that lowering endogenous miR-30 levels might not lead to opposing effects regarding protein production (FIGS. 6D and E).

### Induction of Apoptosis in Tumor Cells and Preadipocytes

To determine whether miRNAs identified in the above described screening exert their specific cellular functions also in cells derived from other species than Chinese Hamster, miR-134-5p, miR-378-5p and let-7d-3p were transiently overexpressed in human cell lines. The examined cell lines comprised tumor cell lines, namely SKOV3 (ovarial carcinoma), T98G (glioblastoma), HCT 116 (colon carcinoma), and the SGBS preadipocytes cell line. As for CHO-SEAP cells, miR-134-5p, miR-378-5p and let-7d-3p induced apoptosis in all four cell lines, with most prominent effect in preadipocytes. These results show that the miRNAs identified in CHO cells to have specific cellular functions are well suitable to induce their specific effects also in cells derived from other species.

Production of Recombinant Adeno-Associated Vectors (rAAVs)

HeLa cells transfected with viral production plasmids can be used to produce viral particles for further infections. To increase the production of recombinant adeno-associated vectors (rAAVs), HeLa cells are co-transfected with rAAV production plasmids and miRNA 483 mimics. This resulted in a 1.5 to 2 fold increase in cellular production of rAAVs (FIG. 11).

### REFERENCES

Aurnhammer, C., Haase, M., Muether, N., Hausl, M., Rauschhuber, C., Huber, I., Nitschko, H., Busch, U., Sing, A., Ehrhardt, A., Baiker, A., (2012) Universal real-time PCR for the detection and quantification of adeno-associated virus serotype 2-derived inverted terminal repeat sequences. Hum Gene Ther Methods 23, 18-28.
Ebert M S, Sharp P A; MicroRNA sponges: progress and possibilities; RNA. 2010 November; 16(11):2043-50
Fischer S, Wagner A, Kos A, Aschrafi A, Handrick R, Hannemann J, Otte K. Breaking limitations of complex culture media: functional non-viral miRNA delivery into pharmaceutical production cell lines. J Biotechnol. 2013 December; 168(4):589-600.
Krämer O, Klausing S, Noll T; Methods in mammalian cell line engineering: from random mutagenesis to sequence-specific approaches; Appl Microbiol Biotechnol. 2010 September; 88(2):425-36.
Mayginnes, J. P., Reed, S. E., Berg, H. G., Staley, E. M., Pintel, D. J., Tullis, G. E., (2006) Quantitation of encapsidated recombinant adeno-associated virus DNA in crude cell lysates and tissue culture medium by quantitative, real-time PCR. J Virol Methods 137, 193-204.
van Rooij E; The art of microRNA research; Circ Res. 2011 Jan. 21; 108(2):219-34.

1. A nucleic acid construct comprising at least two different regions, wherein the regions are selected of
a first region encoding for at least one miRNA stimulating cellular production of a biomolecule in a cell, the miRNA is selected from group 1,
a second region encoding for at least one miRNA and/or mi-RNA inhibitor suppressing cell death, the miRNA is selected from group 2 and the miRNA-inhibitor inhibits a miRNA selected from group 3, and
a third region encoding for at least one miRNA and/or miRNA-inhibitor regulating cell proliferation, the miRNA is selected from group 4 or 5 and the miRNA-inhibitor inhibits a miRNA selected from group 4 or 5,
wherein group 1 consists of SEQ ID NO.: 69, 1, 2, 3, 4, 6, 8, 9, 10, 11, 12, 13, 14, 15, 16, 17, 18, 19, 20, 21, 25, 26, 27, 28, 29, 32, 34, 37, 39, 40, 41, 42, 44, 45, 46, 47, 48, 49, 51, 52, 53, 55, 56, 57, 58, 62, 63, 66, 67, 70, 75, 76, 77, 80, 81, 82, 83, 86, 88, 90, 91, 93, 98, 99, 100, 101, 102, 103, 104, 106, 109, 110, 111, 112, 113, 114, 115, 116, 117, 118, 120, 121, 128, 130, 132, 134, 136, 137, 138, 141, 142, 143, 147, 151, 152, 155, 158, 163, 165, 171, 174, 182, 183, 185, 186, 188, 190, 201, 203, 207, 211, 212, 214, 217, 222, 223, 228, 230, 233, 238, 239, 240, 254, 255, 269, 276, 278, 279, 285, and 294;
group 2 consists of SEQ ID NO.: 1, 2, 3, 4, 6, 8, 9, 20, 52, 98, 5, 7, 24, 31, 33, 50, 54, 59, 60, 61, 64, 68, 71, 73, 74, 79, 85, 87, 89, 92, 94, 95 97, 145, 153, 154, 156, 159, 161, 166, 167, 168, 169, 170, 172, 175, 176, 178, 179, 180, 181, 184, 191, 192, 194, 195, 196, 197, 199, 200, 204, 205, 206, 208, 209, 210, 213, 216, 219, 220, 221, 224, 225, 226, 227, 229, 231, 236, 237, 241, 242, 243, 245, 247, 248, 251, 252, 253, 256, 257, 258, 259, 260, 262, 265, 266, 268, 271, 272, 273, 274, 277, 280, 282, 284, 289, 293, and 295;
group 3 consists of SEQ ID NO.: 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 605, 607, 608, and 609;
group 4 consists of SEQ ID NO.: 5, 7, 68, 79, 94, 10, 11, 12, 13, 14, 15, 16, 17, 18, 19, 22, 23, 30, 35, 36, 38, 43, 65, 72, 78, 84, 96, 105, 107, 108, 119, 122, 123, 124, 125, 126, 127, 129, 131, 133, 135, 139, 140, 144, 146, 148, 149, 150, 160, 162, 164, 173, 177, 187, 189, 193, 198, 202, 215, 218, 232, 234, 235, 244, 246, 249, 250, 261, 263, 264, 267, 270, 275, 281, 283, 287, 288, and 291;
and group 5 consists of SEQ ID NO.: 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, and 606.

2. The nucleic acid construct according to claim 1, wherein the first region encodes for at least one miRNA selected from group 9, and/or the second region encodes for at least one miRNA selected from group 10 and/or an miRNA-inhibitor inhibiting a miRNA selected from group 11, and/or the third region encodes for at least one miRNA selected from group 12 or 13 and/or an miRNA-inhibitor inhibiting a miRNA selected from group 12 or 13,
wherein group 9, consists of SEQ ID NO.: 1, 3, 6, 8, 10, 29, 39, 40, 47, 49, 51, 55, 91, 103, 115, 132, 137, 171, 211 and 294; group 10 consists of SEQ ID NO.: 3, 7, 20, 54, 59, 73, 94, 145, 159, 175, 176, 178, 179, 199, 206, 248, 251, 252, 266 and 272; group 11 consists of SEQ ID NO.: 297, 305, 307, 311, 312, 313, 321, 330, 331, 335, 336, 340, 345, 351, 359, 405, 412, 458, 510 and 608; group 12 consists of SEQ ID NO.: 5, 7, 22, 30, 35, 43, 68, 72, 78, 84, 96, 146, 148, 160, 173, 177, 198, 202, 232, 234, 244, 267 and 283; and group 13 consists of SEQ ID NO.: 517, 523, 526, 529, 531, 533, 537, 548, 550, 558, 560, 561, 563, 566, 567, 571, 575, 577, 583, 591, 600, 601 and 604.

3. The nucleic acid construct according to claim 1, wherein the at least two different regions are controlled by different promoters, preferably at least one promoter is inducible or inhibitable.

4. The nucleic acid construct according to claim 1, wherein the miRNA inhibitor is an antagomir, a miRNA sponge or a miRNA decoy.

5. The nucleic acid construct according to claim 1, wherein the biomolecule is a biopharmaceutical, preferably a recombinant molecule, more preferred a recombinant protein or a recombinant virus.

6. The nucleic acid construct according to claim 1, wherein the nucleic acid construct is an expression vector, an episomal vector or a viral vector.

7. A cell comprising a construct according to claim 1.

8. The cell according to claim 7, wherein the construct is integrated into the cell's genome.

9. The cell according to claim 7, wherein a region of the cell's genome encoding for at least one miRNA selected from group 1, 2, or 4 is amplified and/or a region of the cell's genome encoding for at least one miRNA selected from group 3 or 5 is deleted or silenced.

10. The cell according to claim 7, wherein the cell is stable cell line cell.

11. Method for increasing the yield of a biomolecule produced by a cell cultured in vitro comprising at least two steps selected of
stimulating cellular production of the biomolecule by increasing the level of at least one miRNA selected from group 1 in the cell,
reducing cell death by increasing the level of at least one miRNA selected from group 2 in the cell and/or decreasing the level of at least one miRNA selected from group 3, and
regulating proliferation of the cell by increasing the level of at least one miRNA selected from group 4 or 5 in the cell and/or decreasing the level of at least one miRNA selected from group 4 or 5,
wherein group 1 consists of SEQ ID NO.: 69, 1, 2, 3, 4, 6, 8, 9, 10, 11, 12, 13, 14, 15, 16, 17, 18, 19, 20, 21, 25, 26, 27, 28, 29, 32, 34, 37, 39, 40, 41, 42, 44, 45, 46, 47, 48, 49, 51, 52, 53, 55, 56, 57, 58, 62, 63, 66, 67, 70, 75, 76, 77, 80, 81, 82, 83, 86, 88, 90, 91, 93, 98, 99, 100, 101, 102, 103, 104, 106, 109, 110, 111, 112, 113, 114, 115, 116, 117, 118, 120, 121, 128, 130, 132, 134, 136, 137, 138, 141, 142, 143, 147, 151, 152, 155, 158, 163, 165, 171, 174, 182, 183, 185, 186, 188, 190, 201, 203, 207, 211, 212, 214, 217, 222, 223, 228, 230, 233, 238, 239, 240, 254, 255, 269, 276, 278, 279, 285, and 294;
group 2 consists of SEQ ID NO.: 1, 2, 3, 4, 6, 8, 9, 20, 52, 98, 5, 7, 24, 31, 33, 50, 54, 59, 60, 61, 64, 68, 71, 73, 74, 79, 85, 87, 89, 92, 94, 95 97, 145, 153, 154, 156, 159, 161, 166, 167, 168, 169, 170, 172, 175, 176, 178, 179, 180, 181, 184, 191, 192, 194, 195, 196, 197, 199, 200, 204, 205, 206, 208, 209, 210, 213, 216, 219, 220, 221, 224, 225, 226, 227, 229, 231, 236, 237, 241, 242, 243, 245, 247, 248, 251, 252, 253, 256, 257, 258, 259, 260, 262, 265, 266, 268, 271, 272, 273, 274, 277, 280, 282, 284, 289, 293, and 295;
group 3 consists of SEQ ID NO.: 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 605, 607, 608, and 609;
group 4 consists of SEQ ID NO.: 5, 7, 68, 79, 94, 10, 11, 12, 13, 14, 15, 16, 17, 18, 19, 22, 23, 30, 35, 36, 38, 43, 65, 72, 78, 84, 96, 105, 107, 108, 119, 122, 123, 124, 125, 126, 127, 129, 131, 133, 135, 139, 140, 144, 146, 148, 149, 150, 160, 162, 164, 173, 177, 187, 189, 193, 198, 202, 215, 218, 232, 234, 235, 244, 246, 249, 250, 261, 263, 264, 267, 270, 275, 281, 283, 287, 288, and 291;
and group 5 consists of SEQ ID NO.: 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, and 606.

12. The method of claim 11, wherein the level of a miRNA of group 1, group 2, group 4 or group 5 is increased by overexpressing the miRNA of group 1, group 2, group 4 or group 5 in the cell, by electroporating the cell in the presence of the miRNA of group 1, group 2, group 4 or group 5 or by adding the miRNA of group 1, group 2, group 4 or group 5 and a transfectant to a medium, in which the cell is cultured.

13. The method of claim 11, wherein the level of a miRNA of group 3, group 4 or group 5 is decreased by deleting the region of the cell's genome encoding for the miRNA of group 3, group 4 or group 5 or regulating its transcription, by expressing a miRNA-inhibitor in the cell directed against the miRNA of group 3, group 4 or group 5, by electroporating the cell in the presence of the miRNA-inhibitor or by adding a miRNA-inhibitor and a transfectant to a medium, in which the cell is cultured.

14. Method for producing a biomolecule in a cell comprising the steps
propagating the cell in a cell culture,
increasing the level of at least one miRNA selected from the group consisting of SEQ ID NO.: 1, 2, 3, 4, 5, 6, 7, 8, 9, 10, 11, 12, 13, 14, 15, 16, 17, 18, 19, and 20, and
isolating the biomolecule from the cell culture.

15. A combination of at least one miRNA selected from group 1, at least one miRNA selected from group 2 and/or at least one miRNA-inhibitor inhibiting a miRNA selected from group 3, and at least one miRNA selected from group 4 or 5 and/or at least one miRNA-inhibitor inhibiting a miRNA selected from group 4 or 5, for producing a biomolecule in a cell wherein group 1 consists of SEQ ID NO.: 1, 2, 3, 4, 6, 8, 9, 10, 11, 12, 13, 14, 15, 16, 17, 18, 19, 20, 21, 25, 26, 27, 28, 29, 32, 34, 37, 39, 40, 41, 42, 44, 45, 46, 47, 48, 49, 51, 52, 53, 55, 56, 57, 58, 62, 63, 66, 67, 69, 70, 75, 76, 77, 80, 81, 82, 83, 86, 88, 90, 91, 93, 98, 99, 100, 101, 102, 103, 104, 106, 109, 110, 111, 112, 113, 114, 115, 116, 117, 118, 120, 121, 128, 130, 132, 134, 136, 137, 138, 141, 142, 143, 147, 151, 152, 155, 158, 163, 165, 171, 174, 182, 183, 185, 186, 188, 190, 201, 203, 207, 211, 212, 214, 217, 222, 223, 228, 230, 233, 238, 239, 240, 254, 255, 269, 276, 278, 279, 285, and 294;
group 2 consists of SEQ ID NO.: 1, 2, 3, 4, 6, 8, 9, 20, 52, 98, 5, 7, 24, 31, 33, 50, 54, 59, 60, 61, 64, 68, 71, 73, 74, 79, 85, 87, 89, 92, 94, 95 97, 145, 153, 154, 156, 159, 161, 166, 167, 168, 169, 170, 172, 175, 176, 178, 179, 180, 181, 184, 191, 192, 194, 195, 196, 197, 199, 200, 204, 205, 206, 208, 209, 210, 213, 216, 219, 220, 221, 224, 225, 226, 227, 229, 231, 236, 237, 241, 242, 243, 245, 247, 248, 251, 252, 253, 256, 257, 258, 259, 260, 262, 265, 266, 268, 271, 272, 273, 274, 277, 280, 282, 284, 289, 293, and 295;
group 3 consists of SEQ ID NO.: 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 605, 607, 608, and 609;
group 4 consists of SEQ ID NO.: 5, 7, 68, 79, 94, 10, 11, 12, 13, 14, 15, 16, 17, 18, 19, 22, 23, 30, 35, 36, 38, 43, 65, 72, 78, 84, 96, 105, 107, 108, 119, 122, 123, 124, 125, 126, 127, 129, 131, 133, 135, 139, 140, 144, 146, 148, 149, 150, 160, 162, 164, 173, 177, 187, 189, 193, 198, 202, 215, 218, 232, 234, 235, 244, 246, 249, 250, 261, 263, 264, 267, 270, 275, 281, 283, 287, 288, and 291;
and group 5 consists of SEQ ID NO.: 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, and 606.
